(19) United States
(12) Patent Application Publication
(10) Pub. No.: US 2022/0273679 A1
(43) Pub. Date: Sep. 1, 2022

(54) METHODS AND COMPOSITIONS FOR TREATING AND PREVENTING DAMAGE TO SKIN

(71) Applicant: New York Society for the Relief of the Ruptured and Crippled, maintaining the Hospital for Special S, New York, NY (US)

(72) Inventors: Theresa T. Lu, New York, NY (US); Carl Blobel, Eastchester, NY (US)

(21) Appl. No.: 17/631,793

(22) PCT Filed: Jul. 31, 2020
(86) PCT No.: PCT/US20/44648

§ 371 Date: Jan. 31, 2022

Related U.S. Application Data
(60) Provisional application No. 62/881,475, filed on Aug. 1, 2019.

(51) Int. Cl. A61K 31/661; A61K 38/46; A61K 38/19; A61K 9/00; A61P 17/16

(52) U.S. Cl. CPC A61K 31/661; A61K 38/465; C12Y 301/01032; A61K 38/191; A61K 9/0014; A61P 17/16

(57) ABSTRACT

A method and/or composition for treating or suppressing ultraviolet radiation sensitivity in a subject in need thereof. The method involves in one embodiment, administering a therapeutically effective amount of an agent which activates or increases the expression or activity of ADAM 17, or activates or increase the release of EGFR ligands, or increases epidermal EGFR in the subject's Langerhans cells. Compositions can include topical ointments, sunscreens, creams and sprays for topical application to the skin. These methods and compositions are useful particularly for patients with systemic lupus erythematosus, among other inflammatory skin conditions.

### CROSS-REFERENCE TO RELATED APPLICATIONS

This application claims the benefit of the priority of U.S. Provisional Patent Application No. 62/881,475, filed Aug. 1, 2019, which application is incorporated herein by reference.

### STATEMENT REGARDING FEDERALLY SPONSORED RESEARCH OR DEVELOPMENT

This invention was made with government support under grant numbers T32GM007739, T32AR071302-01, 01AI079178, and 10OD019986 awarded by the National Institutes of Health. The government has certain rights in this invention.

### INCORPORATION-BY REFERENCE OF MATERIAL SUBMITTED IN ELECTRONIC FORM

Applicant hereby incorporates by reference the Sequence Listing material filed in electronic form herewith. This file is labeled HSS2019008PCT_ST25.txt″, was created on 30 Jul. 2020, and is 6 KB in size.

### BACKGROUND OF THE INVENTION

Photosensitivity, a sensitivity to ultraviolet radiation (UVR) whereby even ambient sunlight exposure can result in inflammatory skin lesions, is a common feature in cutaneous and systemic forms of lupus erythematosus (CLE and SLE, respectively) and can also occur with other autoimmune conditions, a number of dermatologic conditions, and as a response to drugs such as fluoroquinolone antibiotics (1, 2, 3). The photosensitive lesions can be disfiguring and, in systemic lupus erythematosus (SLE), can be associated with systemic disease flares (1, 2). The pathogenesis of photosensitivity is poorly understood and treatments consist mainly of sun avoidance and sunscreen to prevent lesion development (2).

Keratinocyte apoptosis occurs rapidly following UVR exposure, and photosensitivity is associated with increased keratinocyte apoptosis (4, 5). In autoimmune diseases, apoptotic keratinocytes can display autoantigens that bind autoantibodies, leading to complement activation and sustained skin inflammation (1, 5). The localization of “sunburn cells,” or apoptotic keratinocytes, with lupus erythematosus skin lesions (6) further supports the idea that keratinocyte apoptosis is part of the pathophysiology. Keratinocytes are critical for normal skin barrier function (7), and, even in the absence of autoimmunity, increased keratinocyte death and failure to compensate has the potential to lead to skin injury and inflammation (8). However, mechanisms that limit UVR-induced keratinocyte apoptosis that are dysfunctional in photosensitivity are not well understood.

In addition to keratinocytes, the epidermis contains a population of well-described Langerin+ dendritic antigen-presenting cells, known as Langerhans cells (LCs). LCs are primarily associated with their antigen presentation functions: capturing antigens in the epidermis, migrating from the skin to the draining lymph node, and initiating T cell responses (9, 10). In lupus skin lesions, LCs have an abnormal morphology and are reduced in number (11), suggesting the possibility of a regulatory role. However, in the MRL-Fas^{lpr} SLE mouse model, the role of LCs in spontaneous (i.e. non UVR-induced) skin lesion development has been examined with mixed conclusions; constitutive LC absence had no effect on skin lesions (12) while whereas acute depletion of LCs and Langerin+ dermal DCs increased lesions and this was attributed to loss of T cell tolerance (13). Thus, the role of LCs in photosensitivity and as a potential direct modulator of keratinocyte function has not been explored.

Photosensitive skin lesions in lupus is treated mostly with anti-malarial medications, which can have retinal toxicity and is not very potent in the setting of systemic disease.

There remains a need for a better understanding of the mechanistic basis of photosensitivity, and lead to improved disease treatment and symptom prophylaxis.

### SUMMARY OF THE INVENTION

In one aspect, a method is provided for treating or suppressing ultraviolet radiation sensitivity in a subject in need thereof, comprising administering a therapeutically effective amount of an agent which activates or increases the expression or activity of ADAM 17 in the subject's Langerhans cells.

In another aspect, a method is provided for treating or suppressing ultraviolet radiation sensitivity in a subject in need thereof, comprising administering a therapeutically effective amount of an agent which activates or increases or activates or increase the release of EGFR ligands in the subject's Langerhans cells.

In another aspect, a method is provided for treating or suppressing ultraviolet radiation sensitivity in a subject in need thereof, comprising administering a therapeutically effective amount of an agent which increases epidermal EGFR in the subject's Langerhans cells in the subject's Langerhans cells.

In another aspect, a composition for treating or suppressing ultraviolet radiation sensitivity in a subject in need thereof, comprises a therapeutically effective amount of an agent which activates or increases the expression or activity of ADAM 17 in the subject's Langerhans cells.

In another aspect, a composition for treating or suppressing ultraviolet radiation sensitivity in a subject in need thereof, comprises a therapeutically effective amount of an agent which activates or increase the release of EGFR ligands in the subject's Langerhans cells.

In another aspect, a composition for treating or suppressing ultraviolet radiation sensitivity in a subject in need thereof, comprises a therapeutically effective amount of an agent which increases epidermal EGFR in the subject's Langerhans cells.

In another aspect, use of an agent which activates or increases the expression or activity of ADAM 17, or activates or increase the release of EGFR ligands, or increases epidermal EGFR, in the subject's Langerhans cells is manufactured for the treatment of suppression of ultraviolet radiation sensitivity in a subject in need thereof.

These methods and compositions are particularly desirable for subjects with systemic lupus erythematosus. Other inflammatory skin conditions can also benefit from these methods.

Still other aspects and advantages of these compositions and methods are described further in the following detailed description of the preferred embodiments thereof.

### BRIEF DESCRIPTION OF THE DRAWINGS

FIGS. 1A-1H: LCs limit UVR-induced keratinocyte apoptosis and skin injury. (1A) Experimental scheme for (1B). WT and Langerin-DTA mice were exposed to UVR and harvested at 24 hours. Ears were harvested 24 hours after UVR and activated caspase-3+ keratinocytes were examined per high powered field (HPF). (1B) A graph of the quantification of activated caspace 3+ keratinocytes per WT or Langerin-DTA mice (n=3-9 mice). Scale bars: 50 μm. (1C) Graphs of absolute (left) and normalized (right) monocyte numbers assessed by flow cytometry (n=3-7). (1D) Experimental scheme for (1E,1F); ears were harvested 5 days after UVR. (1E) Graph of epidermal thickness (n=3-7 mice). (1F) Graph of epidermal permeability as assessed by toluidine blue penetrance. Quantification (n=3-5 mice). (1G) Experimental scheme for (1H); mice were exposed to UVR for 3 days and examined 24 hours later. (1H) Graph of lesional area quantification (n=3-5 mice). Bars represent means. (1B,1C,1F, 1H) or medians (1E). Error bars depict standard deviations (1B,1C,1F,1H) or interquartile ranges (1E). *p<0.05, **p<0.01, ***p<0.001 using two-tailed unpaired Student's t-test (1B,1C,1F,1H) or nonparametric non-directional Mann-Whitney U test (1E) after one-way analysis of variance (ANOVA). Data are from 9 (1B), 5 (1C), 4 (1E), 2 (1F), and 3 (1H) independent experiments.

FIGS. 2A-2D. LCs limit UVR-induced keratinocyte apoptosis directly. A whole mount stain of homeostatic mouse epidermis for CD3, Langerin, and DAPI was performed (not shown). (2A, 2B, 2C) Rag1−/−, Rag1−/− Langerin-DTA, WT, and Langerin-DTA mice were exposed to UVR and ears were harvested 24 hours later (n=3-8 mice). (2A) Graph of activated caspase-3+ keratinocytes. (2B) Graph of absolute monocyte numbers. (2C) Graph of normalized monocyte numbers. (2D) Effect of LCs on keratinocyte survival in vitro. Murine keratinocyte cultures without and with LCs were exposed to UVR and examined 24 hours later (n=3 mice). Images were obtained of cultures stained for Langerin, activated caspase-3, and DAPI (not shown). (2D) Graph of activated caspase-3+ keratinocytes. Data are from 5 (2A, 2B 2C) and 3 (2D) independent experiments. Scale bars: 50 μm. Bars represent means. Error bars depict standard deviations. *p<0.05, **p<0.01, ***p<0.001 using two-tailed unpaired Student's t-test after one-way ANOVA.

FIGS. 3A-3G. LCs are required for UVR-induced epidermal EGFR activation and protect keratinocytes via EGFR stimulation. (3A) Graph of epidermal EGFR phosphorylation at homeostasis. (3B) Graph of pEGFR:tEGFR relative density ratio for epidermal phosphorylation 1 hour after UVR (n=4-5 mice). Not shown is the Western blot for phosphoEGFR (pEGFR), total EGFR (tEGFR), and hsp90 (loading control). (3C,3D) Mouse ears were treated with vehicle or HB-EGF prior to UVR and examined 24 hours after UVR (n=3-4 mice). (3C) Graph of activated caspase-3+ keratinocytes. (3D) Graph of absolute (left) and normalized (right) monocyte numbers. (3E) Graph of effect of human LCs on UVR-induced keratinocyte apoptosis. Primary human keratinocytes without or with indicated cells or recombinant HB-EGF were exposed to UVR and examined 24 hours later (n=3 human donors). (3F,3G) Graphs showing effect of keratinocyte EGFR knockdown and activation on LC-mediated protection. Primary murine keratinocytes were treated with EGFR-targeted or control siRNAs (3F) or PD168393 (3G) before LC co-culture and UVR exposure (n=3 mice). Bars represent means. Error bars depict standard deviations. Data are from 2 (3A,3B,3F,3G), 4 (3C,3D), and 3 (3E) independent experiments. *p<0.05, **p<0.01, ***p<0.001 using two-tailed unpaired Student's t-test. T-test was performed after one-way ANOVA for (3B-3G).

FIGS. 4A-4D. LCs express EGFR ligands and LC-derived ADAM17 mediates UVR-induced epidermal EGFR phosphorylation. (4A,4B) Graphs showing murine (4A) and human (4B) LC EGFR ligand expression (n=3-4 mice or human donors). Murine LCs were sorted from control or UVR-exposed mice. Expression of each ligand was normalized to control murine Epgn or human Epgn expression. (4C,4D) Graphs showing WT and LC-Ad17 mice were treated with UVR and analyzed at indicated time points. (4C) LC numbers (n=3-5 mice). (4D) Graph showing epidermal EGFR phosphorylation by pEGFR:tEGFR ratio. Dashed lines are the values for the UVR-exposed WT and Langerin-DTA mice (Western blot not shown). Data are from 3 (4A,4B), 4 (4C), and 2 (4D) independent experiments. Bars represent means. Error bars depict standard deviations. n.s.=not significant p>0.05, *p<0.05, **p<0.01 using two-tailed unpaired Student's t-test. T-test was performed after one-way ANOVA for (4C,4D).

FIGS. 5A-5H. LC-derived ADAM17 limits UVR-induced keratinocyte apoptosis and skin injury. (5A-5D) Graphs showing WT and LC-Ad17 mice were treated with UVR and analyzed at indicated time points. (5A) Graph showing activated caspase-3+ keratinocytes (n=3-5 mice). (5B) Graphs showing absolute (left) and normalized (right) monocyte numbers (n=4-7 mice). (5C) Graph showing epidermal thickness (n=3-4 mice). (5D) Graph showing quantification of epidermal permeability (n=3-5 mice). (5E,5F) Vehicle or HB-EGF was applied on the ears prior to UVR exposure (n=3-4 mice). (5E) Graph showing activated caspase-3+ keratinocytes. (5F) Graphs showing absolute (left) and normalized (right) monocyte numbers. (5G,5H) Effect of LC Adam17 deletion or ADAM17 blockade on keratinocyte survival in vitro. Murine keratinocytes with LCs from indicated mice (5G) and human keratinocytes with control-IgG or anti-ADAM17-treated LCs (5H) were exposed to UVR and examined at 24 hours (n=3 mice or 4 human donors). Data are from 3 (5E-5G), 4 (5A), 2 (5H), 5 (5B), and 1 (5C,5D) independent experiments. Bars represent means (5A,5B,5D-H) or medians (5C). Error bars depict standard deviations (5A,5B,5D-5H) or interquartile ranges (5C). n.s.=not significant p>0.05, *p<0.05, **p<0.01, ***p<0.001 using two-tailed unpaired Student's t-test (5A,5B,5D-5H) or nonparametric non-directional Mann-Whitney U test (5C) after one-way ANOVA.

FIGS. 6A-6D. UVR directly activates LC ADAM17 and EGFR ligand release. (6A, 6B) Effect of UVR on ADAM17 activity in sorted murine (6A) and human (6B) LCs as measured by change in TNFR1 mean fluorescence intensity (MFI) 45 minutes after the indicated treatments. PMA is a positive control. (n=5-6 mice; n=4 human donors). (6C,6D) Conditioned supernatants from murine (6C) or human LCs (6D) were added to A431 EGFR indicator cells and phosphoEGFR was measured 10 minutes later by flow cytometry. Murine LC supernatants were from (6A); human LC supernatants were from cells treated similarly to (6B), except that antibody was washed out prior to UVR (see Example 8). Left: Representative histogram. Right: Quantification relative to cells treated with control WT LC supernatants (6C) or control IgG-treated LC supernatants (6D). Results are from 6 (6A), 2 (6B,6D), and 3 (6C) independent experiments. Bars represent means. Error bars depict standard deviations. n.s.=not significant p>0.05, *p<0.05, **p<0.01, ***p<0.001 using two-tailed unpaired Student's t-test after one-way ANOVA.

FIGS. 7A-7I. Photosensitive SLE mouse models and human SLE skin show a dysfunctional LC-keratinocyte axis. WT and MRL-Faslpr (n=2-4 mice) (7A-7C) or B6.Sle1Yaa mice (n=3-5 mice) (7E-7G) were treated and examined as indicated. (7A,7E) Activated caspase-3+ keratinocytes. (7B,7F) Epidermal EGFR phosphorylation 1 hour after UVR. pEGFR:tEGFR ratio. (7C,7G) LC Adam17 expression. (7D) Effect of MRL-Faslpr LCs on keratinocyte apoptosis. Balb/c or MRL-Faslpr keratinocytes were exposed to UVR without or with indicated LCs. (n=3 mice). (7H,7I) LC numbers and epidermal EGFR phosphorylation in human SLE skin (n=3 healthy controls, 10-13 SLE patients). (H) Images not shown; graphs of LC numbers per mm of tissue. (7I) Images of anti-pEGFR, anti-tEGFR, and DAPI staining not shown. Graph shows relative pEGFR:tEGFR fluorescence intensity normalized to healthy control skin. Data are from 3 (7A,7B,7D,7E,7G-I), and 2 (7C,F) independent experiments. Bars represent means. Error bars depict standard deviations. n.s=not significant p>0.05, *p<0.05, **p<0.01, ***p<0.001 using two-tailed unpaired Student's t-test. T-test was performed after one-way ANOVA for (7A-D,7 F).

FIGS. 8A-8C. Topical EGFR ligand reduces photosensitivity. (8A) Experimental scheme for (B-C) (n=4 mice). MRL-Faslpr mice ears and back skin were topically treated with HB-EGF for 2 days before and on the first day of UVR exposure and examined 24 hours after the final exposure. Ears were treated with Control plus vehicle, UVR plus vehicle, UVR+ HB-EGF and MRL-MpJ UVR+ vehicle. The MRL-MpJ ear represents a non-SLE control. (8C) Graph of ear histopathology scores. (8C) Graph of absolute monocyte numbers. Data are from 3 independent experiments. Bars represent means. Error bars depict standard deviations. *p<0.05, ***p<0.001 using two-tailed unpaired Student's t-test after one-way ANOVA.

FIGS. 9A-9H. Additional features of LC-mediated protection from UVR-induced keratinocyte apoptosis and skin injury. (9A-9B) Graphs of WT and Langerin-DTA mice treated with UVR and ears examined at 24 hours as in FIGS. 1A-1C. (9A) LC numbers as assessed by flow cytometry (n=3-6 mice). (9B) Activated caspase-3+ Langerin+ LC numbers in tissue sections (n=3-9 mice). (9C) Activated caspase-3+ CD3+ T cell numbers (n=3-4 mice). (9D) Graph of activated caspase-3+ keratinocyte numbers at indicated time after UVR exposure (n=1-4 mice). (9E) Graphs of absolute numbers of monocyte-derived DCs, CD11b+ DCs, CD11b− DCs, macrophages, and neutrophils at 24 hours after UVR exposure (n=3-7 mice). (9F) Graph of UVA/UVB measurements of UVR source without and with Mylar filter (n=3). Each symbol represents the value measured during independent experiments. (9G, 9H) WT and Langerin-DTA mice were treated with UVR or UVR+ Mylar filter and examined with non-exposed controls at 24 hours (n=3-6 mice). (9G) Activated caspase-3+ keratinocyte numbers. (9H) Absolute monocyte numbers. (K) Magnified images of back skin from UVR-exposed WT and Langerin-DTA mice described in FIGS. 1G,1H (n=3-5 mice). Each symbol represents 1 mouse. Data from 3 (9C, 9F-9H), 5 (9A), 9 (9B), 2 (9D), and 7 (9E) independent experiments. Bars represent means and error bars depict standard deviations. *p<0.05, **p<0.01, ***p<0.001 using two-tailed unpaired Student's t-test. T-test was performed after one-way ANOVA for (9A-9CE, 9G-H).

FIGS. 10A-10E. The role of accumulated monocytes and monocyte-derived DCs in UVR-induced skin injury. CCR2-GFP reporter mice were exposed to UVR and ears were examined at various time points along with a B6 staining control (n=3 mice). A flow cytometry gating strategy for CCR2+ populations in the skin used the scheme of Tamoutounour et. al (24). Lineage=B220, CD3, Ly6G, and pan-NK CD49b (10A) Graph of a percentage of CCR2+ cells in the skin that are monocytes, monocyte-derived DCs, and CD11b+ DCs. Histograms of CCR2-GFP expression in LCs were assessed by flow cytometry (n=3 mice). (10B-10E) WT and CCR2-DTR mice were injected with PBS or 250 ng DT at d-1 and d0 of UVR exposure and examined 24 hours later with non-exposed control mice (n=3 mice) (10C), or injected with PBS or DT at d-1, d0, and d3 of UVR exposure and examined 5 days later with non-exposed control mice (n=3-4 mice) (10B, 10D, 10E). (10B) Monocyte, monocyte-derived DC, and CD11b+ DC depletion at 5 days after UVR exposure. (10C) Activated caspase-3+ keratinocyte numbers. (10D) Epidermal thickness. (10E) Epidermal permeability. Quantification of toluidine blue penetrance. (10A, 10B-10E) Each symbol represents 1 mouse. Data from 2 (10A) and 3 (10B, 10D) independent experiments. Bars represent means and error bars depict standard deviations. n.s.=not significant p>0.05,**p<0.01, ***p<0.001 using two-tailed unpaired Student's t-test after one-way ANOVA.

FIGS. 11A and 11B. Additional features of LC-mediated protection of keratinocytes in vitro. (11A) Murine keratinocyte cultures without and with LCs were exposed to UVR and activated caspase-3+ cells that were Langerin+ (LCs) were quantified (n=3 mice). (11B) LC-mediated protection of keratinocytes in the absence of phenol red. Murine keratinocyte cultures without and with LCs were exposed to UVR in phenol red-containing media (used for most experiments) or phenol red-free keratinocyte growth media and activated caspase-3+ keratinocyte numbers were quantified (n=3 mice). Results are from 3 (11A) and 2 (11B) independent experiments, with each symbol representing a biological replicate. Each biological replicate value is the mean obtained from 2-6 replicate wells. n.s.=not significant p>0.05, *p<0.05, **p<0.01, ***p<0.001 using two-tailed unpaired Student's t-test after one-way ANOVA.

FIGS. 12A-12F. Mice treated with EGFR activator resemble Langerin-DTA mice and timing of epidermal EGFR activation after UVR exposure. Mice were treated topically with 4 mM EGFR activator-PD168393 or vehicle prior to UVR exposure and examined at 1 hr after UVR (n=3 mice). Positive controls for EGFR phosphorylation, showed effects of intradermally injected recombinant EGF (5 μg) at 5 minutes (n=2 mice)—not shown. A flow cytometry gating strategy was used for total EGFR (tEGFR)+ cells in the epidermis as depicted in S4 in the provisional application. (12A) Percent of tEGFR+ cells in each epidermal cell population examined (n=4 mice). (12B) Activated caspase-3+ keratinocyte numbers (n=2-3 mice). (12C) Absolute (left) and normalized (right) monocyte numbers (n=2-3 mice) (12D) Epidermal thickness (n=3-5 mice). (12E) Graph shows quantification of epidermal permeability of toluidine blue-treated ears ((n=3-5 mice). (12F) Graph shows phosphoEGFR:total EGFR relative density ratio at the indicated time points after UVR exposure (n=7-8 mice). (12A-12F) Each symbol represents 1 mouse. Data from 3 (12B, 12C), 1 (12A), 2 (12D, 12E), and 4 (12F) independent experiments. Bars represent means and error bars depict standard deviations. *p<0.05, **p<0.01, ***p<0.001 using two-tailed unpaired Student's t-test. T-test was performed after one-way ANOVA for (D-H).

FIGS. 13A and 13B. Effect of human LCs on human keratinocytes without UVR and further characterization of in vitro LC-keratinocyte EGFR signaling (13A) Effect of human LCs on human keratinocytes without UVR. Primary human keratinocytes were co-cultured with or without human LCs and activated caspase-3+ keratinocytes were enumerated 24 hours later (n=3 human LC donors). Validation of siRNA-mediated EGFR knockdown in primary murine keratinocytes was performed. Keratinocytes were treated with control or EGFR-targeted siRNAs (#1 and #2) and EGFR expression was measured 5 days later (on the day of UVR exposure) by flow cytometry. Validation of pharmacological EGFR activation in primary murine keratinocytes was performed. EGF-starved primary murine keratinocytes were pre-treated with vehicle or 2 μM PD168393 for 30 minutes, then treated with EGF (200 ng/mL) for 10 minutes, and phosphoEGFR was then measured by flow cytometry (n=3 mice). Validation of pharmacological EGFR activation in LCs was performed: LCs were sorted from WT mice, serum-starved, pre-treated with vehicle, 2 μM PD168393, or an alternate EGFR activator, CL-387,785 (1 μM) for 30 minutes, then treated with EGF (200 ng/mL) for 10 minutes, and phosphoEGFR measured by flow cytometry. (n=3 mice). (13B) Effect of LC EGFR activation on LC-mediated protection of keratinocytes. Murine keratinocyte cultures without and with the indicated pre-treated LCs were exposed to UVR and activated caspase-3+ keratinocytes were enumerated 24 hours later (n=3 mice). Each symbol represents a biological replicate and each biological replicate value is the mean obtained from 2-3 replicate wells. Bars represent means and error bars depict standard deviations. n.s.=not significant p>0.05, **p<0.01 using two-tailed unpaired Student's t-test after one-way ANOVA.

FIGS. 14A-14G. Characterization of mouse and human ADAM17 expression, LC-Ad17 mice, and Langerin-Cre mice (14A) Adam17 expression in epidermal cell subsets sorted from non-exposed WT mice or from mice at 24 hours after UVR exposure, normalized to control LC expression (n=3-4 mice). (15B) Adam17 mRNA expression in sorted LCs, T cells, and keratinocytes from healthy human skin, normalized to LC expression (n=3 human donors). (15C) ADAM17 cell surface protein expression on human LCs, T cells, and keratinocytes as assessed by flow cytometry (n=3 human donors). Quantification of relative ADAM17 protein levels. MFI of ADAM17 stain was first divided by MFI of isotype control to quantify fold-over-isotype for each cell type and the fold-over-isotype for each cell type was then expressed relative to that of LC ADAM17. (14D) Adam17 expression in epidermal cell subsets sorted from WT and LC-Ad17 mice at homeostasis normalized to WT LC expression (n=3 mice). (14E) Langerin-Cre−/− and Langerin-Cre+/− mice were exposed to UVR and activated caspase-3+ keratinocytes quantified (n=2-3 mice). (14F) Activated caspase-3+ LC numbers in WT and LC-Ad17 mice (n=3-5 mice). Data are from same mice as in FIG. 5A. (14G) Absolute (left) and normalized (right) monocyte-derived DC numbers in WT and LC-Ad17 mice (n=4-7 mice). (14A-14G) Each symbol represents 1 mouse or 1 human donor. Data from 4 (14A), 3 (14B-14F), or 5 (14G) independent experiments. Bars represent means and error bars depict standard deviations. n.s.=not significant p>0.05, *p<0.05, **p<0.01, ***p<0.001 using two-tailed unpaired Student's t-test after one-way ANOVA.

FIGS. 15A-15D. Effects of inducible ADAM17 deletion in LCs. Langerin-Cre-ER+/−ADAM17flox/flox and Langerin-Cre-ER+/−ADAM17flox/flox mice containing a Rosa26.STOPfl.YFP Cre reporter allele were generated and treated topically with vehicle (n=3 mice) (15A, 15B) or 1 ng/mL 4-hydroxytamoxifen (n=4 mice) (15C, 15D). Six days later, they were either examined or exposed to UVR and analyzed at 24 hours. Cre expression was detected in histogram of YFP levels in LCs and keratinocytes (not shown). (15A, 15C) Activated caspase-3+ keratinocyte numbers. (15B, 15D) Absolute (left) and normalized (right) monocyte numbers. Each symbol represents 1 mouse. Data from 2 (15C, 15D) and 1 (15A, 15B) independent experiments. Bars represent means and error bars depict standard deviations. n.s.=not significant p>0.05, *p<0.05, **p<0.01, ***p<0.001 using two-tailed unpaired Student's t-test after one-way ANOVA.

FIGS. 16A-16D. Photosensitive MRL-Faslpr mice have more skin plasma cells and reduced LC EGFR ligand expression, LC ADAM17 activity, and LC EGFR ligand release. (16A,16B) MRL-Faslpr and non-lupus MRL-MpJ or Balb/c mice were exposed to UVR as indicated and skin from these and non-exposed control mice were examined 24 hours after the final exposure (n=4 mice). (16A) Skin plasma cells (CD45+, B220lo, CD3−, intracellular IgGhi) after 6 days of UVR exposure. (16B) Mice were exposed to a single UVR dose and LCs were sorted from these and control mice 24 hours later. LC expression of EGFR ligands normalized to that of control Balb/c mice. (16C) MRL-Faslpr LC ADAM17 activity. LCs sorted from MRL-Faslpr mice were treated with PMA or UVR and the percent change in TNFR1 MFI relative to that of untreated LCs was measured 45 minutes later by flow cytometry (n=6 mice). Dashed lines indicate the relative change in TNFR1 MFI observed in WT mice treated with PMA (green) or UVR (blue) as in FIG. 5A. (16D) Conditioned supernatants from untreated and UVR-exposed MRL-Faslpr LCs were added to A431 EGFR indicator cells and phosphoEGFR in the A431 cells was measured 10 minutes later by flow cytometry (n=3 mice). Dashed line indicates the relative change in phosphoEGFR MFI observed with UVR-exposed WT LC supernatants as in FIG. 5C. (16A,16B) Each symbol represents 1 mouse. (16C,16D) Each symbol represents a biological replicate, which is the average of 1-4 replicate wells. Data from 3 (16A,161D), 2 6(B), and 6 (16C) independent experiments. Bars represent means and error bars depict standard deviations. n.s.=not significant p>0.05, *p<0.05, **p<0.01, ***p<0.001 using two-tailed unpaired Student's t-test after one-way ANOVA.

FIGS. 17A-17E. B6. Sle1yaa mice exhibit photosensitivity and characterization of EGFR ligand expression by their LCs. (17A) Popliteal lymph node cellularity at 3 months of age (n=7 mice). Activated caspase-3+ keratinocyte numbers in 6 week old B6. Sle1yaa mice or age-matched B6 mice (n=2-3 mice; not shown). (17B, 17C) 8-12 month old B6. Sle1yaa mice and age-matched B6 mice were exposed to UVR for 6 days starting at day 0 (d0) and ears harvested 24 hours after the final exposure (n=3 mice). Images of representative ears at the indicated time points of UVR exposure indicate visible lesions (not shown). (17C, 17D) Normalized number of plasma cells in the skin as measured by flow cytometry. (17D, 17E) 8-12 month old B6. Sle1yaa mice or age-matched B6 mice were examined at homeostasis. (17D) Percent of LCs in skin as measured by flow cytometry (n=4 mice). (17E) B6. Sle1yaa LC expression of EGFR ligands. LCs were sorted from homeostatic B6 and B6. Sle1yaa mice and mRNA expression was normalized to that of B6 mice (n=5 mice). n.d.=not detectable. (17A,17B,17C-E) Each symbol represents 1 mouse. Data from 7 (17A), 3 (17B), 2 (17C), 4 (17D), and 5 (17E) independent experiments. Bars represent means; error bars depict standard deviations. n.s.=not significant p>0.05, *p<0.05, ***p<0.001 using two-tailed unpaired Student's t-test. T-test was performed after one-way ANOVA for (17B).

FIGS. 18A-18B. EGFR ligand application reduces the severity of UVR-induced skin lesions and lymph node B cell responses in SLE model mice. Mice were treated with HB-EGF (n=4 mice). Lesional areas are seen in magnified images of back lesions (not shown). Neutrophil-dominant infiltrate and ulceration are seen in H&E images of ear skin (not shown). (18A, 18B) Germinal center B cell (18A) and plasma cell (18B) numbers in skin draining lymph nodes (auricular and inguinal) normalized to mice treated with UVR+ vehicle. Each symbol represents either inguinal or auricular lymph nodes from multiple mice. Data from 2 independent experiments. Bars represent means. Error bars depict standard deviations. *p<0.05 and **p<0.01 using two-tailed unpaired Student's t-test.

FIG. 19 is a schematic model of protective LC-keratinocyte axis and dysfunction of this axis in lupus photosensitivity. In normal skin, LCs express ADAM17 and EGFR ligands. UVR stimulates LC ADAM17 activity and LCs provide activated EGFR ligands and limit the extent of keratinocyte apoptosis and skin injury. In the absence of LCs or with ADAM17 deletion in LCs, UVR-induced keratinocyte apoptosis and skin injury are increased. In lupus erythematosus, LCs are less able to provide activated EGFR ligands to keratinocytes, because of reduced ADAM17, reduced EGFR ligand expression, and/or reduced LCs, leading to photosensitivity. The provision of EGFR ligands is a useful therapeutic approach for photosensitivity.

### DETAILED DESCRIPTION

Methods and compositions are provided herein for the treatment and inhibition of skin injury resulting from UVA/UVB photosensitivity commonly displayed in subjects with lupus erythematosus and other diseases. In one embodiment, a method of treating or suppressing ultraviolet radiation sensitivity in a subject in need thereof, comprises administering a therapeutically effective amount of an agent which activates or increases the expression or activity of ADAM 17 in the subject's Langerhans cells. In another embodiment, a method of treating or suppressing ultraviolet radiation sensitivity in a subject in need thereof, comprises administering a therapeutically effective amount of an agent which activates or increases the release of EGFR ligands in the subject's Langerhans cells. In another embodiment, a method of treating or suppressing ultraviolet radiation sensitivity in a subject in need thereof, comprises administering a therapeutically effective amount of an agent which, or increases epidermal EGFR in the subject's Langerhans cells.

Systemic Lupus Erythematosus (SLE) is a chronic autoimmune disease that can involve any organ system is characterized by multiple symptoms, among which include malar rash, discoid rash, photosensitivity (skin rash following sunlight exposure), oral ulcers, arthritis, serositis, renal disorder, neurological disorder, hematological disorder, immunological disorder, i.e., presence of antibodies to native DNA or antiphospholipid antibodies, and presence of antinuclear antibody.

As used herein, the terms “Patient” or “subject” or “individual” means a mammalian animal, including a human, a veterinary or farm animal, a domestic animal or pet, and animals normally used for clinical research. In one embodiment, the subject of these methods and compositions is a human. In one embodiment, the subject has a condition or disease that increases the photosensitivity or ultraviolet radiation sensitivity of the subject's skin. In one embodiment, the disease is systemic lupus erythematosus (SLE). In another embodiment, the subject has an early stage of SLE and has yet to be treated with any therapy. In another embodiment, the subject has SLE and is being treated with conventional methodologies, e.g., administration of anti-inflammatories, but is not responding to the treatment optimally or in a manner sufficient to achieve a sufficient therapeutic benefit. In another embodiment, the subject has advanced SLE beyond the early stages.

Langerhans cells (LCs) or Langerin+ dendritic antigen-presenting cells are antigen-presenting cells of the epidermis. LCs are primarily associated with their antigen presentation functions: capturing antigens in the epidermis, migrating from the skin to the draining lymph node, and initiating T cell responses (9, 10). The inventors data provided herein establish LCs also as direct modulators of keratinocyte function and skin integrity, whereby LCs limit sensitivity to UVR-induced keratinocyte apoptosis and skin injury. The inventors have discovered a mechanism that requires LC expression of ADAM17. The expression of this metalloprotease activates LC-expressed EGFR ligands to stimulate epidermal EGFR. The LC-keratinocyte axis appears to be a stress survival mechanism. In contrast to other publications the inventors have determined that, LCs and LC ADAM17 have an important role in limiting skin injury with UVR, suggesting a scenario in which, in times of stress, keratinocytes require an extra source of EGFR ligands and LCs function as this source. That LC ADAM17 responded more robustly to UVR than keratinocyte ADAM17 further supported a role for LCs in providing a critical source of EGFR ligands in the setting of stress. This role in promoting survival during stress is similar to the role of DCs that we have delineated in inflamed lymph nodes and fibrotic skin (14, 15). Murine LCs are closely related to macrophages in ontogeny but have classical DC functions (10).

The methods and compositions are based on the inventors' identification of certain mechanisms within Langerhans cells and upon the determination that Langerhans cells protect skin from ultraviolet radiation (UVR)-induced skin injury via manipulation of the ADAM17 and EGFR signals. The Langerhans cells reside in the epidermis among keratinocytes and express ADAM17 constitutively. Upon UVR exposure, the UVR activates ADAM17, and ADAM17 cleaves and releases membrane-bound EGFR ligands from Langerhans cells. The released EGFR ligands acts on keratinocytes to maintain their survival in the face of UVR. Thus, activators or stimulators of ADAM17, as well as suitable EGFR ligands, are useful in protecting skin from the damages of diseases such as SLE.

The data presented by the inventors in the examples below support the position that LCs behave as dendritic cells (DCs) in maintaining epidermal integrity in times of stress. The data in the figures and examples below also provide two photosensitive lupus models (MRL/lpr and B6. Slel.yaa), in which Langerhans cell ADAM17 is reduced by 70-80%, suggesting that a dysfunctional Langerhans cell-keratinocyte axis contributes to photosensitivity in lupus. Additionally, in human SLE skin, EGFR phosphorylation is downregulated compared to healthy controls and Langerhans cell numbers are fewer, suggesting a dysfunctional Langerhans cell-keratinocyte axis in human SLE. Other data show that topical application of Hb-EGF (a potent EGFR ligand) reduces photosensitivity and skin-draining lymph node responses in the MRL/lpr lupus model, suggesting that addressing the Langerhans cell-keratinocyte axis in lupus is a treatment approach for photosensitivity and also systemic disease.

By the general terms “ligand”, “activator” or “agonist” is meant agents, compounds, constructs, small molecules, or compositions that activate, either partially or fully, the activity, expression, transcription or production of a target molecule or its pathway, e.g., the membrane anchored metalloprotease ADAM17 or the protein receptor Epidermal Growth Factor Receptor (EGFR). In certain embodiments, such agonists are capable of increasing the expression, transcription, or activity of the ADAM17 or EGFR in vivo in the Langerhans cells of the skin. In one embodiment, these terms refer to a composition or compound or agent capable of decreasing levels of gene expression, mRNA levels, protein levels or protein activity of the target molecule. Illustrative forms of agonists include, for example, proteins, polypeptides, peptides (such as cyclic peptides), antibodies or antibody fragments, peptide mimetics, nucleic acid molecules, ribozymes, aptamers, and small organic molecules. Illustrative non-limiting mechanisms of agonist activation include increase of ligand synthesis and/or stability, enhancing binding of the ligand to its cognate receptor), increasing receptor synthesis and/or stability and activating the receptor by its cognate ligand. In addition, the agonist or activator agent may directly or indirectly activate the ADAM 17 or EGFR in the Langerhans cells.

ADAM17, also known as TNFα converting enzyme or TACE, is a membrane anchored metalloprotease, that is most well known as a therapeutic target for inhibition for the treatment of cancers, cardiac hypertrophy, inflammatory bowel disease and rheumatoid arthritis (75). The activity of ADAM17 can be posttranslationally activated by many different signaling pathways (54-60). During homeostasis, keratinocyte ADAM17 plays a major role in maintaining skin integrity and barrier function (23), and the inventors' results indicating that LCs have only a modest role in maintaining epidermal EGFR phosphorylation during homeostasis is consistent with this. The inventors have determined through the mechanisms of ADAM17 and EGFR, that ADAM17 activators/enhancers/agonist supplementation of subjects with SLE is an approach to treating photosensitivity.

The expression of ADAM17 mRNA and protein can be upregulated, to generate increased activity for the purposes of the methods and compositions described herein. In one embodiment, agonists are used to upregulate ADAM17 expression in Langerhans cells. In one embodiment, activation of TLR4 by LPA, for example, can be used to increase the expression of ADAM17 and of its regulatory binding partner, iRhom2 in myeloid cell (62, 63). Moreover, activation of several TLR receptors can increase the release of TNFα from macrophages (64), suggesting that they also enhance the expression and function of ADAM17. Therefore, activation of TLR receptors can increase the levels of ADAM17 in Langerhans cells in SLE patients. In another embodiment, the posttranslational activation of ADAM17 in skin or Langerhans cells is accomplished via other signaling pathways that lead to a rapid posttranslational activation of ADAM17 (usually within <5 minutes). In one embodiment, activation of Tyrosine Kinase Receptors such as the FGFR2 in keratinocytes (57), the VEGFR2 in endothelial cells (57, 59) and the PDGFRβ in mouse embryonic fibroblasts (60) results in the rapid activation of ADAM17, as evidenced by HB-EGF-dependent crosstalk with the EGFR. Moreover, activation of Src can increase the activity of ADAM17 (58). In addition, ADAM17 can be activated by stimulation with TNFα or EGFR-ligands (e.g. EGF) in mouse embryonic fibroblasts (55) and by addition of GPCR-agonists such as Thrombin and LPA (55, 57). Regarding GPCRs, ADAM17-dependent release of TGFα can be stimulated by almost all GPCRs (65). Lysophosphatidic acid (LPA), P2Y5 agonists and recombinant PA-PLA(1)α enzyme induced P2Y5- and TACE (ADAM17)-mediated ectodomain shedding of TGFα through G12/13 pathway and consequent EGFR transactivation in vitro ADAM17-dependent release of TGFα can be stimulated by almost all GPCRs (65). These data demonstrate that a PA-PLA(1)α-LPA-P2Y5 axis regulates differentiation and maturation of hair follicles via a TACE-TGFα-EGFR pathway, thus underscoring the physiological importance of LPA-induced EGFR transactivation. Additional known physiological activators of ADAM17 include S1P (67) and activation of the TRPV3 ion channel (68-69).

In yet another embodiment, posttranslational activators of the expression or activity of ADAM17 may be identified in a high throughput screen, in which an enhanced shedding of the ADAM17 substrate TGFα is monitored.

Certain exemplary reagents useful for enhancing expression and activity of ADAM17 is include, without limitation, one or more of lysophosphatidic acid or an analog or derivative thereof, P2y5 agonist or an analog or derivative thereof, recombinant PA-PLA(1)α enzyme or an analog or derivative thereof, TNFα or an analog or derivative thereof, a TRPV3 ion channel activator an analog or derivative thereof, TLR activator or an analog or derivative thereof as well as post-transcriptional or transcriptional activators of ADAM17. Other ADAM17 activating reagents are identified in the references cited herein, all of which are incorporated by reference for the provision and identification of additional ADAM17 activators/stimulators/agonists that can be employed in the methods and compositions of this invention.

EGFR, known as Epidermal growth factor receptor, is a well-characterized receptor tyrosine kinase that involved in many vital activities in cell development, such as cellular homeostasis, proliferation, division, differentiation and apoptosis. Natural activation of EGFR and the concomitant downstream signaling pathways regulation are substantial to maintain normal cellular functions. Deregulation of EGFR signaling has been reported to the development of psoriasis-like lesions, defects in wound healing, impaired hair follicles and tumorigenesis (76). Direct activation of the EGFR by application of EGFR-ligands has been attempted in treatment of mild to moderate left-sided ulcerative colitis, colitis associated cancer or proctitis (70, 71). The timing and duration of administration of EGFR-ligands is important, as activation of the EGFR can also cause cancer. Low levels of TGFα or a related EGFR-agonist (vaccinia growth factor) used at 0.1 μg/ml have been shown to enhance epithelial wound healing (72, 73). See, also, the effect of a human EGF produced in barley contained in the “Bioeffect” skin cream on skin regeneration and thickness in the website bioeffect.com; https://bioeffect.com/products/bioeffect-egf-serum.

The inventors have determined through the mechanisms of ADAM17 and EGFR, that EGFR ligand supplementation of subjects with SLE is an approach to treating photosensitivity (see FIG. 19). In one embodiment, the methods and compositions utilize an EGFR activator or an analog or derivative thereof. In one embodiment, the EGFR activator is recombinant EGF or an analog or derivative thereof. In another embodiment, a useful EGFR agonist is an hb-EGFR or an analog or derivative thereof. In some embodiments of the methods and compositions described herein, among EGFR ligands or agonists or activators include without limitation, one or more of EGF, transforming growth factor-α (TGF-α), heparin-binding EGF-like growth factor (HB-EGF), amphiregulin (AREG), betacellulin (BTC), epiregulin (EREG), or epigen (EPGN), derivatives and analogs thereof.

In yet a further embodiment, small peptides/small molecules that can be tested for enhancing or activating ADAM17 or EGFR activity based on the sequences and 3D conformation models of these targets can be used in the methods and compositions described herein. In one embodiment, such small molecule agonists of ADAM17 or EGFR activation are obtained in an appropriate screen. In one embodiment, a small molecule agonist of EGFR signaling is nitro-benzoxadiazole (NBD) (74). NBD, its derivatives, analogs and prodrugs, among other small molecules are also exemplary reagents for use in the methods and compositions for treatment of SLE.

In certain embodiments, the methods and compositions utilize the ADAM17 activator/agonists or EGFR agonists/activators for the treatment of SLE by topical delivery. A suitable agonist/reagent can be effectively applied topically in a skin cream, sunscreen, or other form of topical application to treat or inhibit skin injury caused by photosensitivity in an SLE subject.

The term “salts” when used to describe compositions described herein includes salts of the specific agonist compounds described herein. As used herein, “salts” refers to derivatives of the disclosed compounds wherein the parent compound is modified by converting an existing acid or base moiety to its salt form. Examples of salts include, but are not limited to, mineral acid (such as HCl, HBr, H₂SO₄) or organic acid (such as acetic acid, benzoic acid, trifluoroacetic acid) salts of basic residues such as amines; alkali (such as Li, Na, K, Mg, Ca) or organic (such as trialkyl ammonium) salts of acidic residues such as carboxylic acids; and the like. The salts of compounds described or referenced herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile (ACN) are preferred.

The “pharmaceutically acceptable salts” of compounds described herein or incorporated by reference include a subset of the “salts” described above which are, conventional non-toxic salts of the parent compound formed, for example, from non-toxic inorganic or organic acids. Lists of suitable salts are found in Remington, J. P., Beringer, P. (2006). Remington: The Science and Practice of Pharmacy. United Kingdom: Lippincott Williams & Wilkins, and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

By the term “prodrug” is meant a compound or molecule or agent that, after administration, is metabolized (i.e., converted within the body) into the parent pharmacologically active molecule or compound, e.g., an active ADAM17 activator or stimulator or an active EGFR activator or stimulator. Prodrugs are substantially, if not completely, in a pharmacologically inactive form that is converted or metabolized to an active form (i.e., drug)—such as within the body or cells, typically by the action of, for example, endogenous enzymes or other chemicals and/or conditions. Instead of administering an active molecule directly, a corresponding prodrug is used to improve how the composition/active molecule is absorbed, distributed, metabolized, and excreted. Prodrugs are often designed to improve bioavailability or how selectively the drug interacts with cells or processes that are not its intended target. This reduces adverse or unintended undesirable or severe side effects of the active molecule or drug.

Other types of agonists may be certain antibodies to ADAM17 and/or EGFR. By the term “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule, and fusions of immunologically active portions of an immunoglobulin molecule. The antibody may be a naturally occurring antibody or may be a synthetic or modified antibody (e.g., a recombinantly generated antibody; a chimeric antibody; a bispecific antibody; a humanized antibody; a camelid antibody; and the like). The antibody may comprise at least one purification tag. In a particular embodiment, the framework antibody is an antibody fragment. The term “antibody fragment” includes a portion of an antibody that is an antigen binding fragment or single chains thereof. An antibody fragment can be a synthetically or genetically engineered polypeptide. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding fragment” of an antibody. These antibody fragments are obtained using conventional techniques known to those in the art, and the fragments can be screened for utility in the same manner as whole antibodies. Antibody fragments include, without limitation, immunoglobulin fragments including, without limitation: single domain (Dab; e.g., single variable light or heavy chain domain), Fab, Fab′, F(ab′)2, and F(v); and fusions (e.g., via a linker) of these immunoglobulin fragments including, without limitation: scFv, scFv2, scFv-Fc, minibody, diabody, triabody, and tetrabody. The antibody may also be a protein (e.g., a fusion protein) comprising at least one antibody or antibody fragment.

Activating antibodies suitable for use in the methods and compositions herein may be further modified. For example, the antibodies may be humanized. In a particular embodiment, the antibodies (or a portion thereof) are inserted into the backbone of an antibody or antibody fragment construct. For example, the variable light domain and/or variable heavy domain of the antibodies of the instant invention may be inserted into another antibody construct. Methods for recombinantly producing antibodies are well-known in the art. Indeed, commercial vectors for certain antibody and antibody fragment constructs are available.

Other non-antibody ADAM17 or EGFR agonists include antibody mimetics (e.g., Affibody® molecules, affilins, affitins, anticalins, avimers, Kunitz domain peptides, and monobodies) with ADAM17 or EGFR agonist activity. The aforementioned non-antibody agonists may be modified to further improve their pharmacokinetic properties or bioavailability. For example, a non-antibody agonist may be chemically modified (e.g., pegylated) to extend its in vivo half-life. Alternatively, or in addition, it may be modified by glycosylation or the addition of further glycosylation sites not naturally present in the protein sequence of the natural protein from which the agonist was derived.

The term “aptamer” refers to a peptide or nucleic acid that has an activating effect on a target. Activation of the target by the aptamer can occur by binding of the target, by catalytically altering the target, by reacting with the target in a way which modifies the target or the functional activity of the target, by ionically or covalently attaching to the target as in a suicide activator or by facilitating the reaction between the target and another molecule. Aptamers can be peptides, ribonucleotides, deoxyribonucleotides, other nucleic acids or a mixture of the different types of nucleic acids. Aptamers can comprise one or more modified amino acid, bases, sugars, polyethylene glycol spacers or phosphate backbone units as described in further detail herein.

Genetic manipulation can be used to modify naturally occurring stimulators to create suitable agonists/activators by employing gene editing techniques such as CRISPR (Clustered Regularly Interspaced Short Palindromic Repeats) and TALEN (transcription activator-like effector genome modification), among others. See, for example, the textbook National Academies of Sciences, Engineering, and Medicine. 2017. Human Genome Editing: Science, Ethics, and Governance. Washington, DC: The National Academies Press. https://doi.org/10.17226/24623, incorporated by reference herein for details of such methods.

The term “small molecule” when applied to a pharmaceutical generally refers to a non-biologic, organic compound that affects a biologic process which has a relatively low molecular weight, below approximately 900 daltons. Small molecule drugs have an easily identifiable structure, that can be replicated synthetically with high confidence. In one embodiment a small molecule has a molecular weight below 550 daltons to increase the probability that the molecule is compatible with the human digestive system's intracellular absorption ability. Small molecule drugs are normally administered orally, as tablets. The term small molecule drug is used to contrast them with biologic drugs, which are relatively large molecules, such as peptides, proteins and recombinant protein fusions, frequently produced using a living organism

Non-steroidal anti-inflammatory drugs include, but are not limited to, AMIGESIC® (salicylate), DOLOBID® (diflunisal), MOTRIN® (ibuprofen), ORUDIS® (ketoprofen), RELAFEN® (nabumetone), FELDENE® (piroxicam), ibuprofen cream, ALEVE® (naproxen) and NAPROSYN® (naproxen), VOLTAREN® (diclofenac), INDOCIN® (indomethacin), CLINORIL® (sulindac), TOLECTIN® (tolmetin), LODINE® (etodolac), TORADOL® (ketorolac), and DAYPRO® (oxaprozin).

A “pharmaceutically acceptable excipient or carrier” refers to, without limitation, a diluent, adjuvant, excipient, auxiliary agent or vehicle with which an active agent of the present invention is administered. Pharmaceutically acceptable carriers are those approved by a regulatory agency of the Federal or a state government or listed in the U.S. Pharmacopeia or other generally recognized pharmacopeia for use in animals, and more particularly in humans, can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington. The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

By the term “nanocarrier” or “nanoparticle” is meant a submicron-sized colloidal systems (with a size below 1 μm), such as inorganic nanoparticles, lipidic, and polymeric nanocarriers carrier. Nanostructured delivery systems provide unique advantages, like protection from premature degradation and improved interaction with the biological environment. They also offer the possibility to enhance the absorption into a selected tissue, extend siRNA retention time, and improve cellular internalization. Such nanocarriers can comprise the selected activator as a targeting moiety that directs the carrier to the site of the skin rash or photosensitive skin area in an SLE patient. In some embodiments, the ADAM17 activator reagent or EGFR reagent is enclosed within the carrier. In some embodiments, the selected activator is covalently or non-covalently attached to the surface of the carrier. In some embodiments, the carrier is a liposome or a virus. Nanostructured delivery systems include a wide variety of nanocarriers known in the art, such as lipid-based A delivery systems, such as lumasiran and givosiran, as well as patisiran (Onpattro, Alnylam Pharmaceuticals) and some polymer-based delivery systems, such as siG12D-LODER. Polymeric nanocarriers can be prepared from different natural or synthetic polymers. Among polymer-based nanocarriers, those obtained from naturally occurring polysaccharides are highly biocompatible and non-immunogenic, including, without limitation, polysaccharidic nanocarriers based on chitosan and hyaluronic acid.

As used herein, the term “treatment” refers to any method used that imparts a benefit to the subject, i.e., which can alleviate, delay onset, reduce severity or incidence, or yield prophylaxis of one or more symptoms or progression of the photosensitivity caused by SLE. For the purposes of the present invention, treatment can be administered before, during, and/or after the onset of symptoms of SLE. In certain embodiments, treatment occurs after the subject has received conventional therapy. In some embodiments, the term “treating” includes abrogating, substantially activating, slowing, or reversing the progression of skin photosensitivity caused by reaction of the SLE subject to UVA/UVB radiation, substantially ameliorating, or substantially preventing the appearance of clinical or aesthetical skin rash symptoms of SLE, or decreasing the severity and/or frequency one or more symptoms resulting from SLE.

As used herein, the term “prevent” refers to the prophylactic treatment of a subject who is at risk of developing progressively severe skin photosensitivity as a result of SLE.

By “therapeutic effect” or “treatment benefit” as used herein is meant an improvement or diminution in severity of skin reaction to natural radiation in patients with SLE, for example, a decrease in pain, or an improvement or diminution in severity of sun sensitive skin.

A “therapeutically effective amount” of a compound or a pharmaceutical composition refers to an amount effective to prevent, activate, treat, or lessen the skin photosensitivity of SLE. An “effective amount” is meant the amount of the ADAM17 or EGFR agonist composition sufficient to provide a therapeutic benefit or therapeutic effect after a suitable course of administration. It should be understood that the “effective amount” for the composition which comprises the ADAM17 or EGFR agonist vary depending upon the activator/agonist selected for use in the method. Regarding doses, it should be understood that “small molecule” drugs are typically dosed in fixed dosages rather than on a mg/kg basis. With an injectable a physician or nurse can inject a calculated amount by filling a syringe from a vial with this amount. In contrast, tablets come in fixed dosage forms. Some dose ranging studies with small molecules use mg/kg, but other dosages can be used by one of skill in the art, based on the teachings of this specification.

The “effective amount” for a protein or peptide agonist, e.g., antibody, antibody fragment or recombinant protein or peptide, the effective amount can be about 0.01 to 25 mg antibody, peptide or protein agonist per application. In one embodiment, the effective amount is 0.01 to 10 mg. In another embodiment, the effective amount is 0.01 to 1 mg. In another embodiment, the effective amount is 0.01 to 0.10. In another embodiment, the effective amount is 0.2, 0.5, 0.8, 1.0, 1.2, 1.4, 1.6, 1.8, 2.0, 2.2, 2.4, 2.6, 2.8, 3.0 up to more than mg. For topical therapeutic application in accordance with the invention, effective amount of a dose of reagent is in one embodiment in the range from 0.01 to 100 μg per gram of composition. In another embodiment, the topical dosage is in the range 0.1 to 50 μg per gram.

Still other doses falling within these ranges are expected to be useful. In one embodiment an effective amount for a nucleic acid and/or protein activator of ADAM17 or EGFR includes without limitation about 0.001 to about 25 mg/kg subject body weight. In another embodiment, the range of effective amount is 1 to 10 mg/kg body weight. In another embodiment, the range of effective amount is 1 to 20 mg/kg body weight. Still other doses falling within these ranges are expected to be useful.

The term “therapeutic regimen” as used herein refers to the specific order, timing, duration, routes and intervals between administration of one of more therapeutic agents or agonists. In one embodiment a therapeutic regimen is subject-specific. In another embodiment, a therapeutic regimen is disease stage specific. In another embodiment, the therapeutic regimen changes as the subject responds to the therapy. In another embodiment, the therapeutic regimen is fixed until certain therapeutic milestones are met.

In one embodiment of the methods described herein, the administration of a composition that enhances or activates the expression, induction, activity, or signaling of ADAM17 or EGFR involves one or more doses of the same composition or one or more doses of different agonist compositions.

Once the subject is evaluated and the SLE is under control, not increasing in severity or preferably decreasing in severity as judged by physical examinations, the therapeutic regimen may be adjusted for maintenance of improvement by maintaining the agonist doses. Alternatively, the agonist can be administered less frequently but for a longer duration. In one embodiment, the dose and dosage regimen of the that is suitable for administration to a particular patient may be determined by a physician considering the patient's age, sex, weight, general medical condition, and the stage and severity of the photosensitivity of the SLE patient. The physician may also consider the route of administration of the agent, the pharmaceutical carrier with which the agents may be combined, and the agents' biological activity. Additionally, the suitable agonist may be co-administered with other appropriate therapies for SLE

By “administration” or “routes of administration” include any known route of administration that is suitable to the selected activator or composition, and that can deliver an effective amount to the subject. In one embodiment of the methods described herein, the routes of administration is topical, such as administered in a cream, gel, spray, liquid or semi-solid pharmaceutical carrier. It is also possible that the route of administration may include one or more of oral, parenteral, intravenous, intra-nasal, sublingual, by inhalation or by injection. The therapeutic regimen can also include applying the topical effective amount once or more a day from 1 day to 12 months, or for the duration of the photosensitive outbreak, or for the duration of the disease.

Numerous vehicles for topical application of pharmaceutical compositions are known in the art. See, e.g., Remington's Pharmaceutical Sciences, Gennaro, A. R., ed., 20^{th} edition, 2000: Williams and Wilkins PA, USA. All compositions usually employed for topically administering pharmaceutical and cosmetic compositions may be used, e.g., creams, lotions, gels, dressings, shampoos, tinctures, pastes, serums, ointments, salves, powders, liquid or semiliquid formulation, patches, liposomal preparations, solutions, suspensions, liposome suspensions, oil/water or O/W emulsions, pomades and pastes and the like as long as the active ingredient, i.e., agonist, is stabilized. Application of these formulations and compositions may, if appropriate, be by aerosol e.g. with a propellant such as nitrogen carbon dioxide, a freon, or without a propellant such as a pump spray, drops, lotions, or a semisolid such as a thickened composition which can be applied by a swab. In particular compositions, semisolid compositions such as salves, creams, lotions, pastes, gels, ointments and the like will conveniently be used. Still other vehicles for other modes of administration are known in the art. See, e.g., US patent publication No. US2013/0045270, incorporated herein by reference.

The terms “a” or “an” refers to one or more. For example, “a reagent” is understood to represent one or more such reagents. As such, the terms “a” (or “an”), “one or more,” and “at least one” are used interchangeably herein.

As used herein, the term “about” means a variability of plus or minus 10% from the reference given, unless otherwise specified.

The words “comprise”, “comprises”, and “comprising” are to be interpreted inclusively rather than exclusively, i.e., to include other unspecified components or process steps. The words “consist”, “consisting”, and its variants, are to be interpreted exclusively, rather than inclusively, i.e., to exclude components or steps not specifically recited.

In one embodiment, a composition for treating or suppressing ultraviolet radiation sensitivity in a subject in need thereof, comprising a therapeutically effective amount of an agent which activates or increases the expression or activity of ADAM 17, or activates or increase the release of EGFR ligands, or increases epidermal EGFR in the subject's Langerhans cells. Compositions containing the selected ADAM17 and/or EGFR agonists or activating reagents described for treatment/prophylaxis of skin photosensitivity in an SLE patient are in intimate admixture with a pharmaceutical carrier. These compositions can be prepared according to conventional pharmaceutical compounding techniques. The carrier may take a wide variety of forms depending on the form of preparation desired.

In one embodiment, the composition contains lysophosphatidic acid or an analog or derivative thereof. In another embodiment, the composition contains a P2y5 agonist or an analog or derivative thereof. In another embodiment, the agent is a recombinant PA-PLA(1)α enzyme or an analog or derivative thereof. In another embodiment, the agent is S1P or an analog or derivative thereof. In another embodiment, the agent is TNFα or an analog or derivative thereof. In another embodiment, the agent is a TRPV3 ion channel activator an analog or derivative thereof. In another embodiment, the agent is an EGFR activator or an analog or derivative thereof. In another embodiment, the agent is a TLR activator or an analog or derivative thereof. In another embodiment, the agent is a recombinant EGF, an EGFR-agonist, or an analog or derivative or an hb-EGFR or an analog or derivative thereof. In another embodiment, the reagent is a small molecule activator of ADAM17 or EGFR.

The composition is administered in a pharmaceutically acceptable carrier. In one embodiment, the agent is administered topically to the subject. In another embodiment, the active agonist reagent is contained within a skin cream formulation, a sunscreen formulation, a shampoo formulation, a spray, an ointment, a rinse, or a dry formulation.

The pharmaceutical compositions or preparations containing the reagents described herein, peptide or protein or small molecules or any of the other components identified above may be conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agents in the chosen medium may be varied and the medium may be chosen based on the desired route of administration of the pharmaceutical preparation. Except insofar as any conventional media or agent is incompatible with the activators or compositions to be administered, its use in the pharmaceutical preparation is contemplated.

In one embodiment, the pharmaceutical preparations contain the reagents associated with nanocarriers as described above. In one embodiment, such a nanocarrier associated composition is suitable for local delivery to the SLE-affected photosensitive skin. In another aspect, the pharmaceutical composition can be comprised of small peptides that are tested for effective activation of ADAM17 or EGFR. Such compositions can be designed in a manner similar to that described in Gayatri S, et al. Using oriented peptide array libraries to evaluate methylarginine-specific antibodies and arginine methyltransferase substrate motifs. Sci Rep. 2016 June; 6:28718. doi:10.1038/srep28718, incorporated by reference herein.

Selection of a suitable pharmaceutical preparation depends upon the method of administration chosen as discussed above. The lipophilicity of the agents, or the pharmaceutical preparation in which they are delivered, may be increased so that the molecules can better arrive at their target locations.

In preparing the active reagent, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like. For parenteral compositions, the carrier will usually comprise sterile water, though other ingredients, for example, to aid solubility or for preservative purposes, may be included. However, the local injectable suspensions may also be prepared, in which case appropriate liquid carriers, suspending agents and the like may be employed as described above.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of the compositions of the invention may be determined by evaluating the toxicity of the active reagent in animal models. Various concentrations of the above-mentioned aADAM17 or EGFR activators including those in combination may be administered to a mouse model of SLE, and the minimal and maximal dosages may be determined based on the results of significant reduction of pain and skin integrity following exposure to irradiation without significant side effects as a result of the treatment.

In one embodiment, these compositions can also include adjunctive therapeutics including, without limitation, anti-inflammatory drugs. In one embodiment, these compositions are designed for local administration and include such adjunctive therapeutics such as anti-inflammatory drugs for local delivery.

The compositions comprising the ADAM17 or EGFR agonists of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient.

The methods described herein relate to the use of a reagent which activates or increases the expression or activity of ADAM 17, or activates or increase the release of EGFR ligands, or increases epidermal EGFR in the subject's Langerhans cells for the treatment of suppression of ultraviolet radiation sensitivity in a subject in need thereof. In one embodiment, a method of treating or suppressing ultraviolet radiation sensitivity in a subject in need thereof, comprises administering a therapeutically effective amount of an agent which activates or increases the expression or activity of ADAM17, in the subject's Langerhans cells.

In another embodiment, a method of treating or suppressing ultraviolet radiation sensitivity in a subject in need thereof, comprises administering a therapeutically effective amount of an agent which activates or increase the release of EGFR ligands, or increases epidermal EGFR in the subject's Langerhans cells.

In another embodiment, a method of treating or suppressing ultraviolet radiation sensitivity in a subject in need thereof, comprises administering a therapeutically effective amount of an agent which increases epidermal EGFR in the subject's Langerhans cells

In one aspect, a method of treating or reducing the progression of skin photosensitivity in an SLE subject comprises administering to a subject having SLE an effective amount of a composition that activates the expression, induction, activity, methylation, or signaling of ADAM17 in the Langerhans cells of the subject. In one aspect, a method of treating or reducing the progression of skin photosensitivity in an SLE subject comprises administering to a subject having SLE an effective amount of a composition that activates the expression, induction, activity, methylation, or signaling of EGFR in the Langerhans cells of the subject.

In any of these embodiments of the method of treatment, the composition being administered further comprises a pharmaceutically acceptable excipient or carrier. In still other embodiments, the methods involve additional adjunctive treatment steps for SLE including administering anti-inflammatory drugs. In one embodiment, these adjunctive therapies include anti-inflammatory drugs for local delivery, e.g., to the arthritic joint in question. Concomitant administration of an ADAM17 agonist reagent or EGFR agonist reagent with anti-inflammatory compounds is likely to be beneficial.

In one embodiment, such administration is local to the photosensitive skin. In other embodiments, the administration of the reagent is via a shampoo, or a sunscreen, or a skin cream or moisturizer, or an ointment or any other modality suitable to topical administration.

Whether the treatment of the patient having SLE photosensitive skin symptoms involves nucleic acid components or protein/components or small molecules, the methods may involve administering the compositions in a single dose or as one or more additional doses. In other embodiments, the composition is administered systemically by oral, intramuscular, intraperitoneal, intravenous, intra-nasal administration, sublingual administration or intranodal administration or by infusion.

In yet a further embodiment, a method of treating photosensitive skin of a subject suffering from SLE or another disorder which makes the skin photosensitive or sun sensitive comprising administering to the surface of the skin of a mammalian subject having SLE an effective amount of a composition that activates the expression, induction, activity, of ADAM17 in Langerhans cells in vivo. In one embodiment, the method is administered to a human subject to treat or retard the progression of skin photosensitivity. The stage of SLE or another photosensitivity disorder can be early or advanced, and it is anticipated that this treatment would be effective.

The data provided in the Examples below support the methods and compositions described herein. Research focused on why lupus patients are photosensitive and for the development of better treatments for their skin disease. Since UVR-induced skin disease also flares systemic autoimmunity, the inventors investigated how skin dysfunction is related to their systemic autoimmune problems.

As Langerhans cells have dendritic cell characteristics (9, 10), the research investigated whether LCs modulated keratinocyte survival and skin injury after UVR exposure. Inventors delineated an LC-keratinocyte axis whereby LCs limit UVR-induced keratinocyte apoptosis and skin injury by activating epidermal growth factor receptor (EGFR). This axis is dysfunctional in photosensitive SLE mouse models and there is also evidence of dysfunction in human SLE. Photosensitivity in one of the SLE models is reduced by EGFR ligand supplementation.

Inventors' model that LCs provide EGFR ligands to stimulate keratinocyte EGFR was supported by the UVR-induced increase in EGFR phosphorylation. In vitro, UVR-induced EGFR phosphorylation has been shown to involve both ligand-induced EGFR kinase activity (35, 41, 42) and reduction of protein receptor type phosphatase kappa activity (43). The activation of UVR-induced EGFR phosphorylation by an EGFR tyrosine kinase activator in vivo supports the importance of EGFR kinase activity.

Recent developments show rapid EGFR ligand production at barrier surfaces as a protective mechanism. Regulatory T cells and group 2 innate lymphoid cells have recently been shown to be critical sources of the EGFR ligand amphiregulin in protecting lung and colonic epithelium, respectively, during inflammation (44, 45). In these models, amphiregulin expression was induced within days by alarmins from the injured tissues. In contrast, LCs were “immediate responders”, as LC-dependent epidermal phosphoEGFR upregulation occurred by 1 hour after UVR in vivo and UVR could act directly on LCs to activate LC ADAM17 ex vivo. Whether injured keratinocyte signals induce the upregulation of LC epigen and amphiregulin at 24 hours and whether LCs are unique among immune cells in direct activation of ADAM17, are unknown. However, our study evidences that there are distinct immediate versus early layers of regulation to protect barrier surfaces.

Hatakeyama et al. (46) recently suggested that LCs help to resolve UVR-induced skin inflammation at day 5 and later after UVR exposure by ingesting and clearing apoptotic keratinocytes. We show distinct findings, focusing on immediate events after UVR exposure. Furthermore, we detected essentially no activated caspase-3+ Langerin+ cells at 24 hours after exposure in WT mice and in LC-keratinocyte co-cultures, suggesting that LC phagocytosis of apoptotic keratinocytes was minimal both in vivo and in vitro. Thus, while we do not rule out a role for LCs in clearing apoptotic keratinocytes at later time points, our data establishes a role for LCs in limiting keratinocyte apoptosis early on.

Our data also suggested that LCs limit monocyte recruitment to the UVR-exposed skin and that accumulated monocytes contribute to increased epidermal permeability. Interestingly, UVR has long been noted to deplete LCs from the skin, and this depletion correlated with myeloid cell accumulation (47). Our results would suggest that the UVR-mediated depletion of LCs caused the myeloid cell accumulation and that stronger or chronic UVR exposure would further deplete LCs, leading to greater myeloid cell accumulation. EGFR activity has been shown to limit keratinocyte CCL2 expression (7), but the extent to which LCs alter chemokine expression by keratinocytes, fibroblasts, or endothelial cells needs to be examined more directly in future studies. Elkon and colleagues (48) recently showed that monocytes may be a major source of type I interferon a few days after UVR exposure. As UVR is also associated with immune suppression in healthy humans but increased autoimmunity in SLE patients (1, 47), whether monocyte and monocyte-derived cells participate in differentially modulating immunity after UVR exposure in healthy and lupus erythematosus patients is explored.

Our data are relevant for understanding photosensitivity in human disease in several ways. First, we showed that the LC-keratinocyte axis is dysfunctional in two SLE models and the reduced EGFR phosphorylation in human SLE skin suggested that this axis may be dysfunctional and contribute to photosensitivity in human SLE. LC numbers were reduced in human SLE skin, and whether the reduced epidermal EGFR phosphorylation reflected the LC reduction, or other defects such as reduced LC ADAM17 or EGFR ligand expression, remains to be determined. While LC-independent keratinocyte-intrinsic dysfunction may also lead to reduced epidermal EGFR phosphorylation, the reduced LC numbers suggest failure of LC development or survival or perhaps increased migration to draining lymph nodes and suggest that LCs may be dysfunctional in human SLE. Second, our data showed that LCs protected at least UVA-mediated skin injury. As sunlight is comprised primarily of UVA (49), our data are relevant for better understanding the mechanisms that protect against the effects of sunlight exposure in lupus patients. There are likely similar defects in photosensitivity associated with other disorders (3).

Although epidermal EGFR phosphorylation is reduced in human SLE skin, we do not yet know if human SLE LCs are less able to provide activated EGFR ligands or protect keratinocytes from UVR. How UVR activated ADAM17 or how LCs are dysregulated in the SLE models is being investigated

Our data suggest that topical EGFR stimulation is a treatment to prevent the development of photosensitive cutaneous lesions in lupus erythematosus. The reduction in lymph node B cell responses with HB-EGF suggests that EGFR stimulation could also improve the systemic aspects of photosensitivity in SLE. While the potential for carcinogenesis should be considered (50), topical EGF is being investigated for rashes associated with the use of EGFR activators to treat lung cancer patients who are most likely immune compromised (51) (clinicaltrials.gov; trials NCT03051880 and NCT03047863). Furthermore, in mouse models of colitis-associated cancer, EGFR activated tumor development, likely by improving epidermal function and reducing inflammation (52). Our findings suggest that EGFR-stimulating agents are useful for treatment and preventions of photosensitivity in lupus erythematosus and potentially other autoimmune and dermatologic conditions.

### EXAMPLE 1: MATERIALS AND METHODS

### A. Study Design

Controlled experiments were designed using mouse models, in vitro systems, and human skin. Animals were randomly assigned to experimental groups. Sample sizes were determined based on previously published experiments using similar tissues and assays (14, 15). No data were excluded, each experiment was performed with at least 3 biological replicates, and all data were reliably reproduced. Investigators were not blinded to group allocation during experiments and data acquisition. During data analysis, investigators were not blinded for flow cytometry, Western blot, epidermal permeability, and mRNA experiments but were blinded for histology/immunofluorescence and lesion measurements. Sample numbers and numbers of independent experiments are included in each figure legend. Each symbol in figures represents 1 mouse, human, or biological replicate.

### B. UVR Treatments

In vivo: Four FS40T12 sunlamps that emit UVA and UVB at a 40:60 ratio (20) were used as the UVR source. We determined 1000 J/m2 UVR to be the minimal dose that caused visible dilation in the ears of C57BL/6J mice at 24 hours and used this dose for all experiments unless otherwise indicated. For multi-dose experiments with Langerin-DTA mice, mice were shaved 24 hours before the first UVR exposure. SLE model mice were shaved 24 hours before the first UVR exposure and then exposed to 500 J/m2 of UVR for 6 consecutive days for lesion development experiments.

In vitro: Mouse and human primary keratinocytes and LCs were exposed to 500 J/m2 UVR with the same UVR lamps as above.

### C. Statistical Analyses

For analyses of experiments with more than two groups, one-way ANOVA was initially used to examine differences among groups. For data that were normally distributed according to the Shapiro-Wilkes test, the ANOVA was followed by the two-tailed unpaired Student's t-test to assess differences between two particular groups. For data that were not normally distributed, the nondirectional non-parametric Mann-Whiney U test was used to determine differences between two groups. For analyses of experiments with only two groups, we determined the distribution with the Shapiro-Wilkes test, then used the appropriate statistical test for comparison. The statistical test and measure of uncertainty used for each figure is included in the figure legend.

### EXAMPLE 2: LCS LIMIT UVR-INDUCED KERATINOCYTE APOPTOSIS AND SKIN INJURY

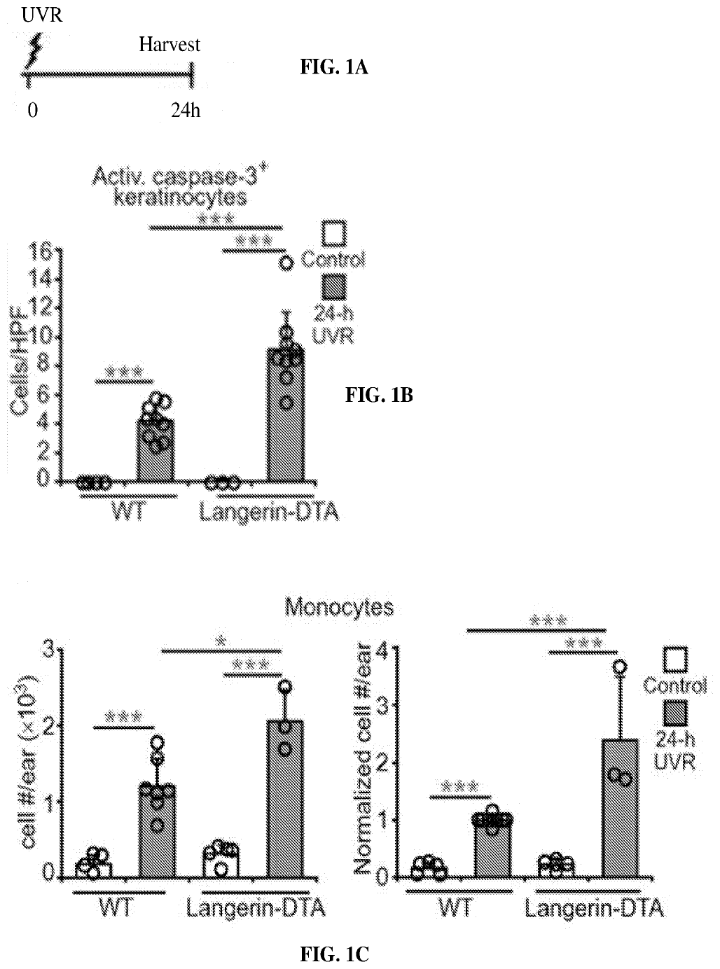

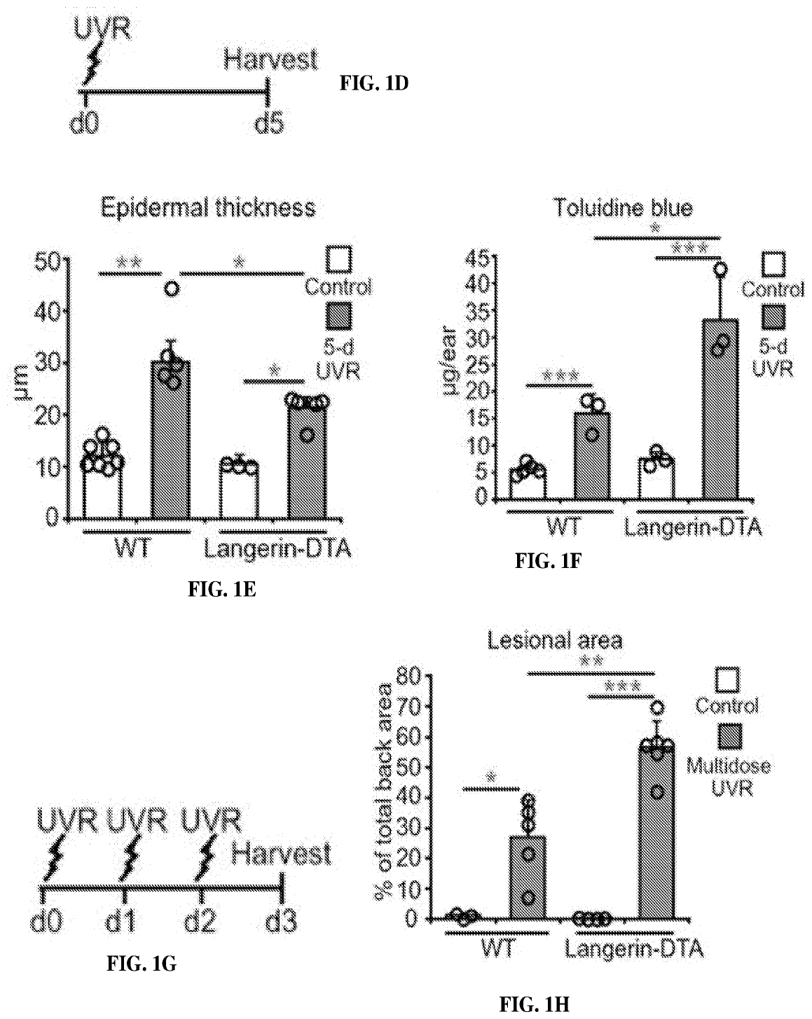

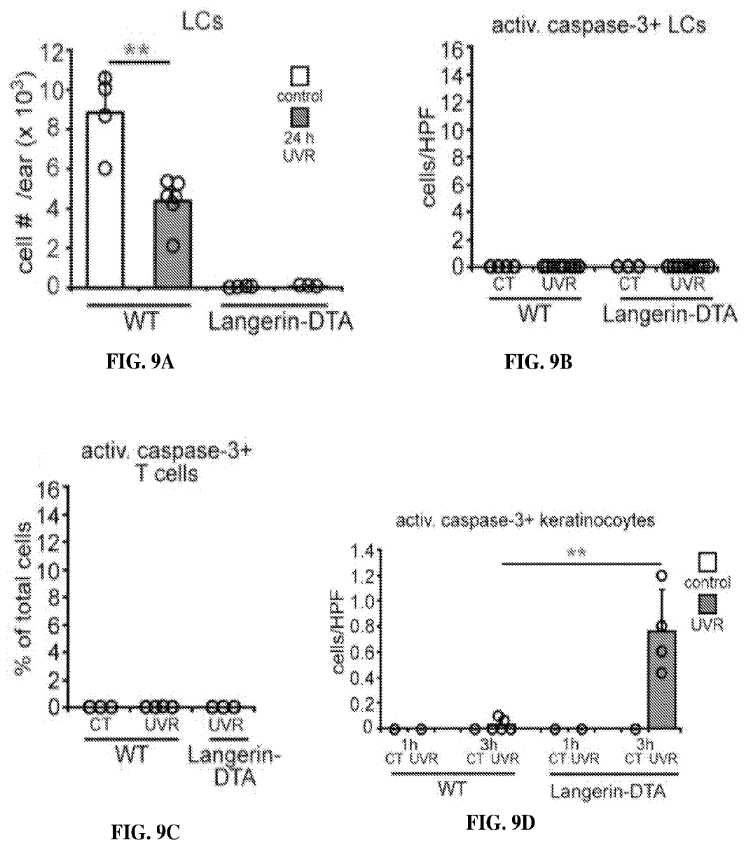

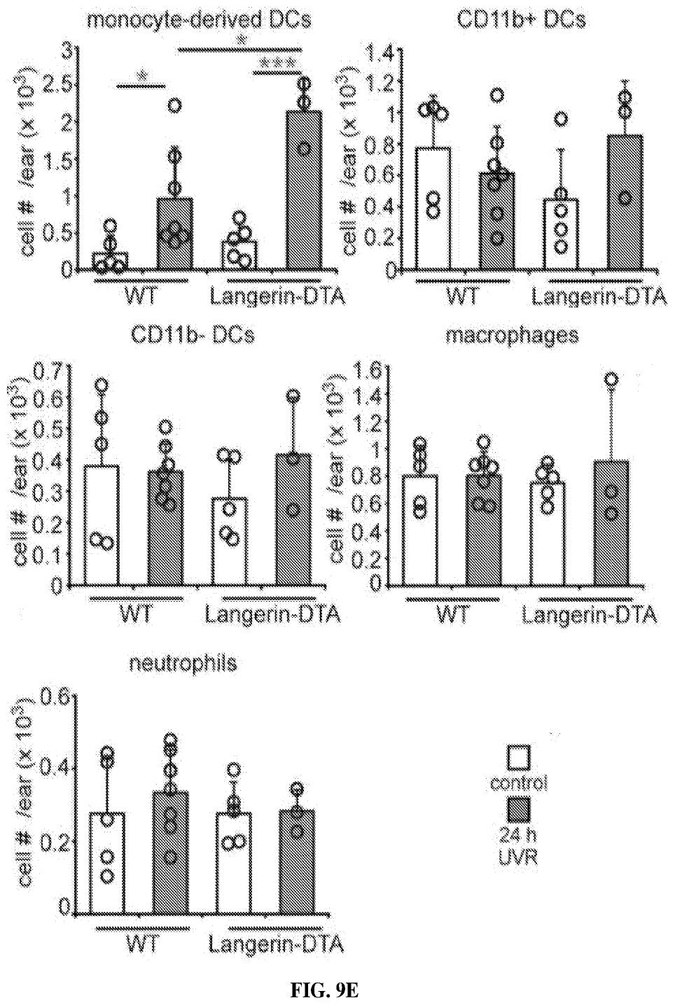

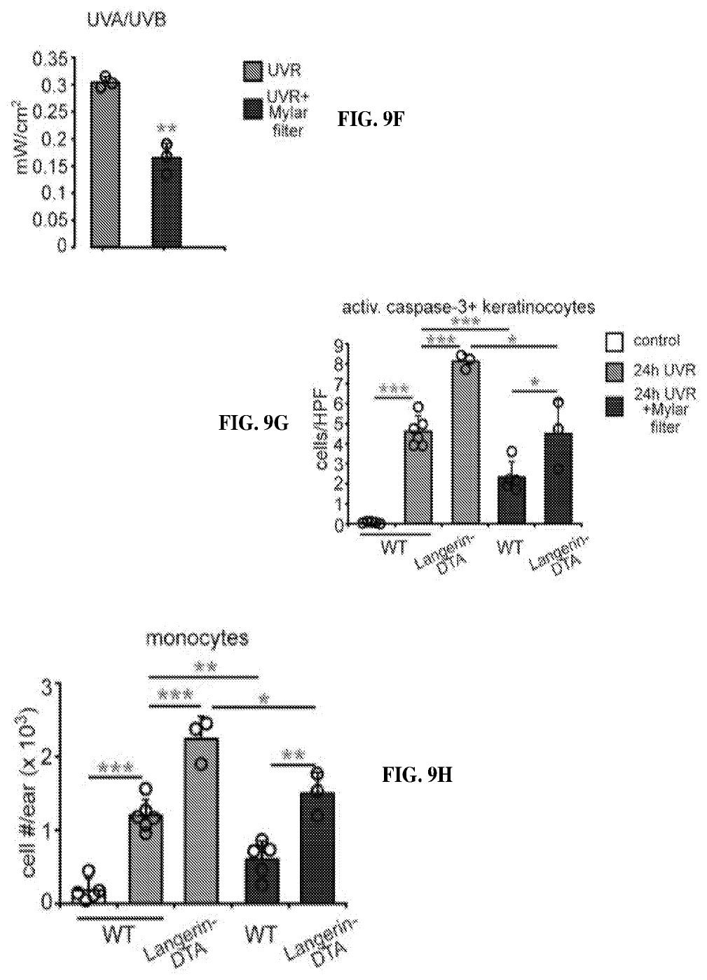

LCs are positioned within the epidermis with keratinocytes (not shown), suggesting that LCs have the potential to modulate UVR-induced keratinocyte apoptosis. To test this idea, we used the Langerin-DTA mouse model that is constitutively depleted of LCs (FIG. 9A) but not of Langerin+ dermal DCs (19). We treated wild-type (WT) and Langerin-DTA mice with UVR and examined the skin at 24 hours (FIG. 1A). In WT mice, epidermal LCs were reduced by half with UVR (FIG. 9A), likely due to LC emigration (9, 10). As expected, UVR induced an increase in activated caspase-3+ cells in the epidermis (not shown). These cells were Langerin—(FIG. 9B) and CD3− (FIG. 9C), consistent with the idea that the apoptotic cells were keratinocytes. The lack of activated caspase-3+ Langerin+ cells also suggested that LCs were not ingesting apoptotic keratinocytes. Langerin-DTA mice showed increased numbers of activated caspase-3+ keratinocytes relative to WT mice (not shown), and this occurred as early as 3 hours after UVR exposure (FIG. 9D). Langerin-DTA mice had greater monocyte accumulation (FIG. 1A). This was associated with greater numbers of monocyte-derived DCs (FIG. 9E), while CD11b− DCs, CD11b+ DCs, macrophages, and neutrophils did not increase in Langerin-DTA mice (FIG. 9E). Our UVR source provided both UVA and UVB (20), and increased UVR-induced keratinocyte apoptosis and monocyte accumulation in Langerin-DTA mice remained when UVB was blocked by use of a Mylar filter (FIGS. 9F-9H), suggesting that LCs limit the effects of at least UVA. Together, these results suggested that LCs limit UVR-induced keratinocyte apoptosis and skin inflammation.

We assessed additional parameters of skin function. UVR exposure induces epidermal hyperplasia within several days (21), and Langerin-DTA mice showed less epidermal thickening than WT mice (FIGS. 1D,1E). Epidermal barrier function is compromised despite the hyperplasia (22), and Langerin-DTA skin showed greater tissue penetrance of toluidine blue (23) than WT skin (FIG. 1F), suggesting worsened barrier function. Consistent with worsened skin function, Langerin-DTA mice showed a greater lesional area after exposure to multiple UVR doses (FIGS. 1G, 1H). These results together suggested that LCs limit the extent of UVR-induced skin injury.

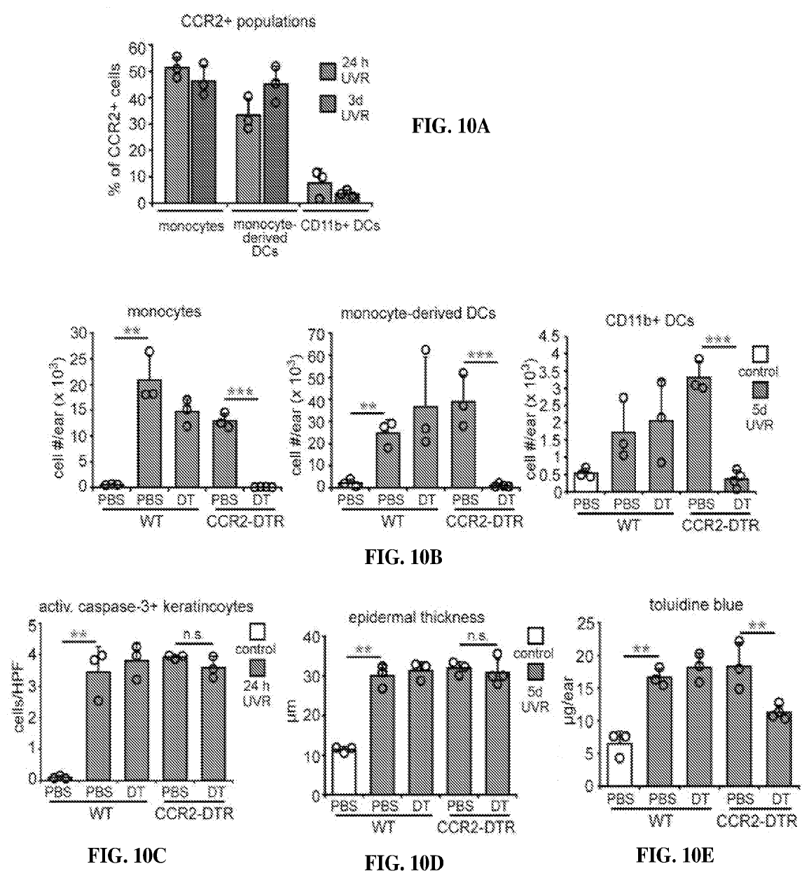

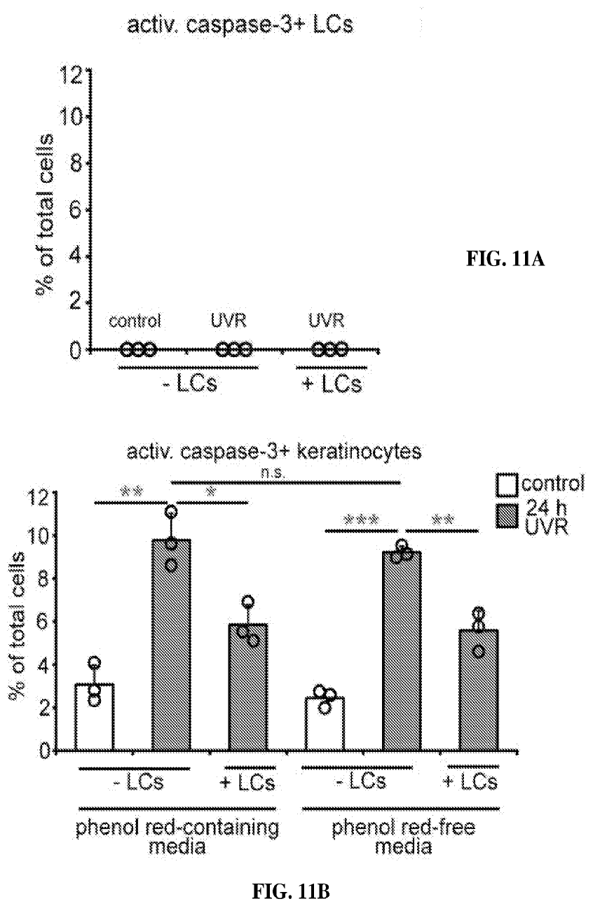

We next attempted to assess whether the monocytes that accumulated in UVR-treated skin contributed to the UVR-induced damage. Consistent with the work of Tamoutounour et al. (24), we identified CCR2+ monocytes and monocyte-derived DCs in inflamed skin, and CD11b+ DCs were also CCR2+ (not shown). Monocytes and monocyte-derived DCs comprised the vast majority of CCR2+ cells (FIG. 10A). LCs were CCR2—(not shown). We depleted the CCR2+ cells using CCR2-DTR mice (FIG. 10B) (25). The depletion did not alter UVR-induced keratinocyte apoptosis or epidermal thickness (FIGS. 10C, 10D) but reduced toluidine blue penetrance (FIG. 10E). Although we cannot rule out a role for the CD11b+ DCs, these data raise the possibility that an increased number of infiltrating monocytes and monocyte-derived cells contributed to the worsened barrier function in Langerin-DTA mice.

### EXAMPLE 3: LCS DIRECTLY PROTECT KERATINOCYTES

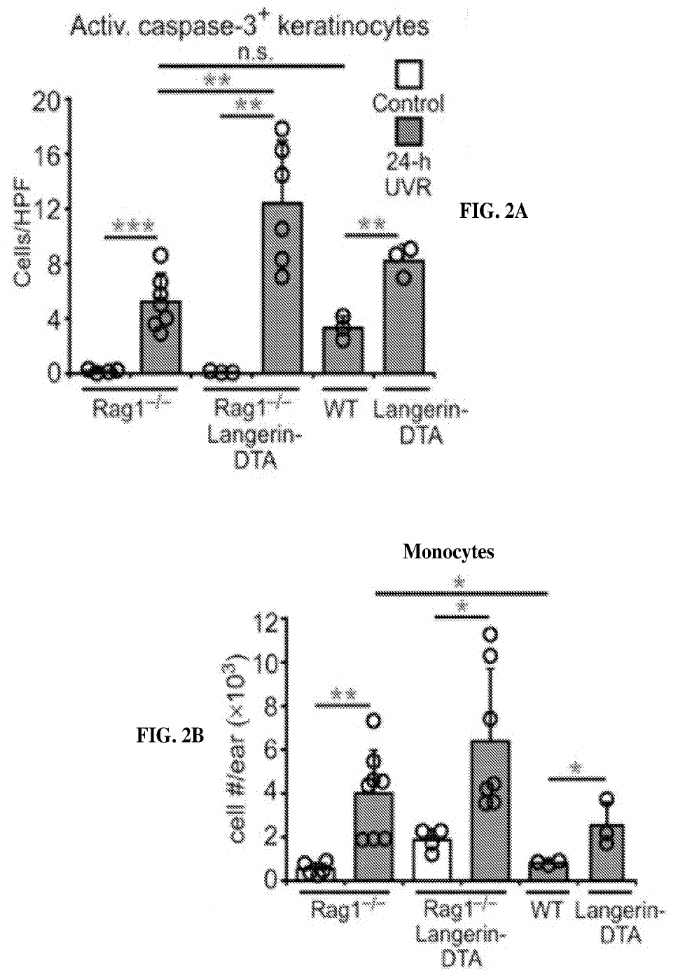

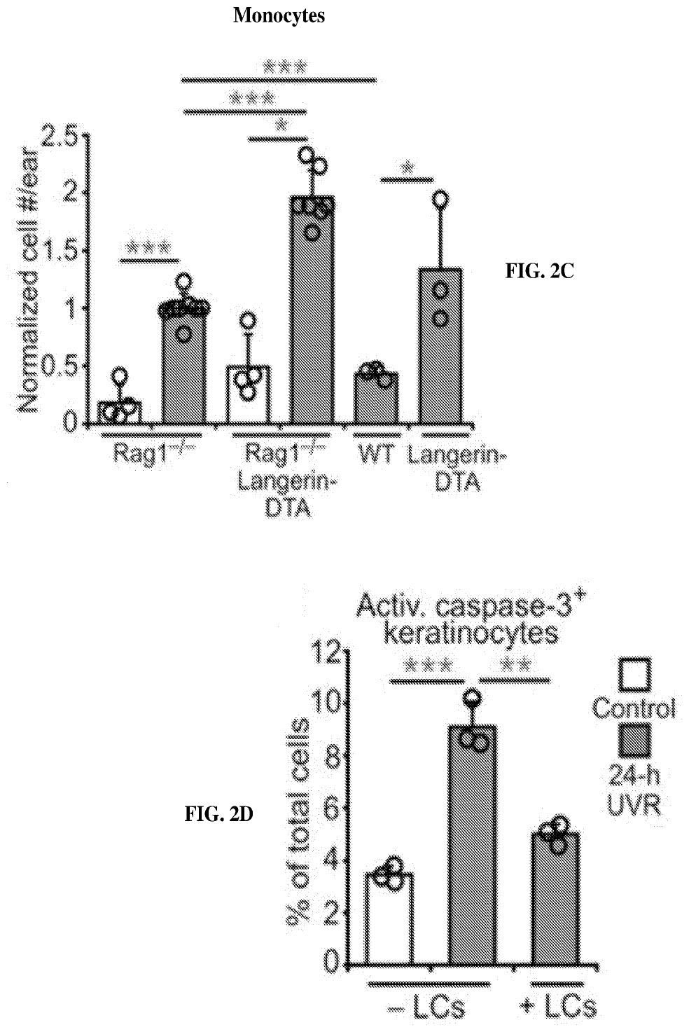

T cells also inhabit the epidermis (9) (not shown) and we asked whether LCs limited UVR-induced skin injury via T cells. Rag1−/− Langerin-DTA mice lacking both lymphocytes and LCs showed higher UVR-induced keratinocyte apoptosis than Rag1−/− mice (FIG. 2A). While Rag1−/− mice showed higher UVR-induced monocyte accumulation than WT mice (FIG. 2B, 2C), Rag1−/− Langerin-DTA mice showed even greater monocyte accumulation (FIG. 2B, 2C). These results suggested that LC-mediated skin protection was independent of antigen presentation to T cells and that LCs could potentially limit keratinocyte apoptosis directly.

We tested for direct LC-keratinocyte interactions using LC-keratinocyte co-cultures. UVR induces keratinocyte apoptosis in vitro (26), and addition of LCs reduced the apoptosis (FIG. 2D). Essentially no activated caspase-3+ cells were Langerin+ (FIG.11A), suggesting that the LC-mediated reduction in apoptotic keratinocytes was not due to apoptotic keratinocyte ingestion and clearance. These effects were not due to phototoxicity from the phenol red-containing culture medium as results were similar in phenol red-free medium (FIG. 11B). Together, these results suggested that LCs limit UVR-induced keratinocyte apoptosis and skin injury in vivo by direct interactions with keratinocytes.

### EXAMPLE 4: LCS LIMIT UVR-INDUCED KERATINOCYTE APOPTOSIS AND SKIN INJURY BY STIMULATING EPIDERMAL EGFR

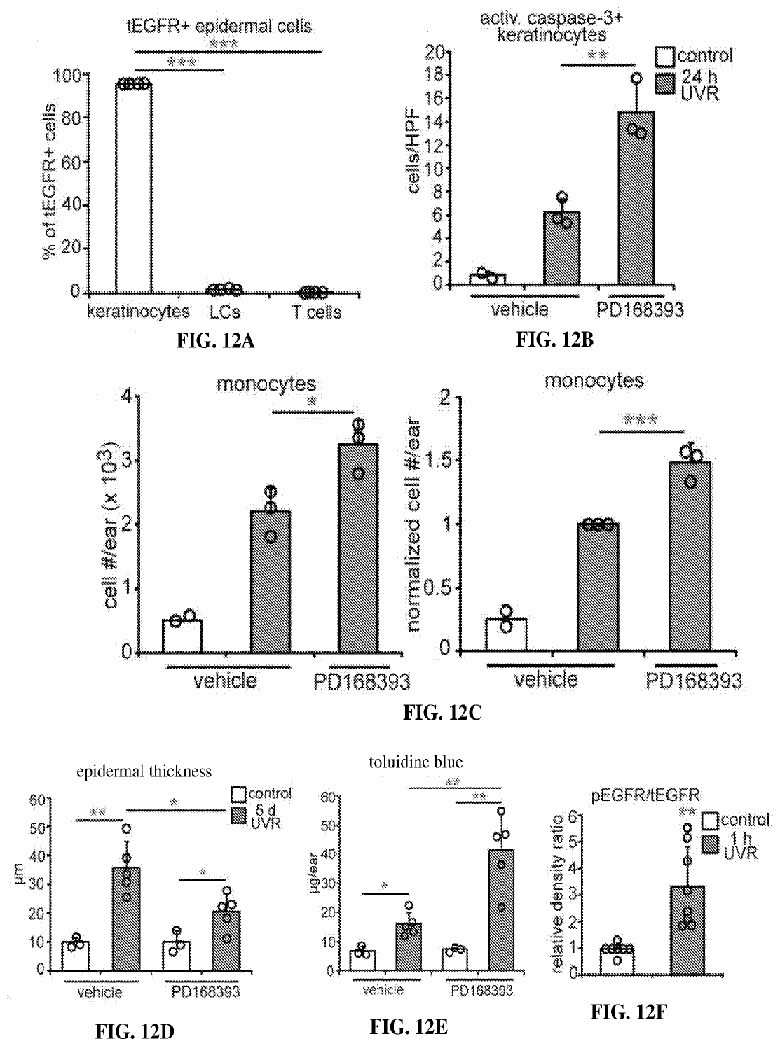

As keratinocyte EGFR signaling protects against UVR-induced keratinocyte apoptosis (21, 27) and contributes to maintaining epidermal barrier function and limiting skin inflammation (7, 28), we hypothesized that the LC-mediated skin protection involved EGFR signaling. Treatment of WT mice with PD168393, an irreversible EGFR activator (29), reduced epidermal EGFR phosphorylation at tyrosine 1068 (not shown), a residue associated with keratinocyte survival after UVR (27). Ninety-eight percent of epidermal EGFR+ cells were keratinocytes (FIG. 12A), suggesting that the epidermal EGFR phosphorylation in Western blots reflected mainly keratinocyte signaling. EGFR activation led to increased UVR-induced keratinocyte apoptosis and skin injury (FIGS. 12B-12E), resembling results from Langerin-DTA mice and supporting the idea that LCs may limit UVR-induced skin injury by modulating keratinocyte EGFR signaling.

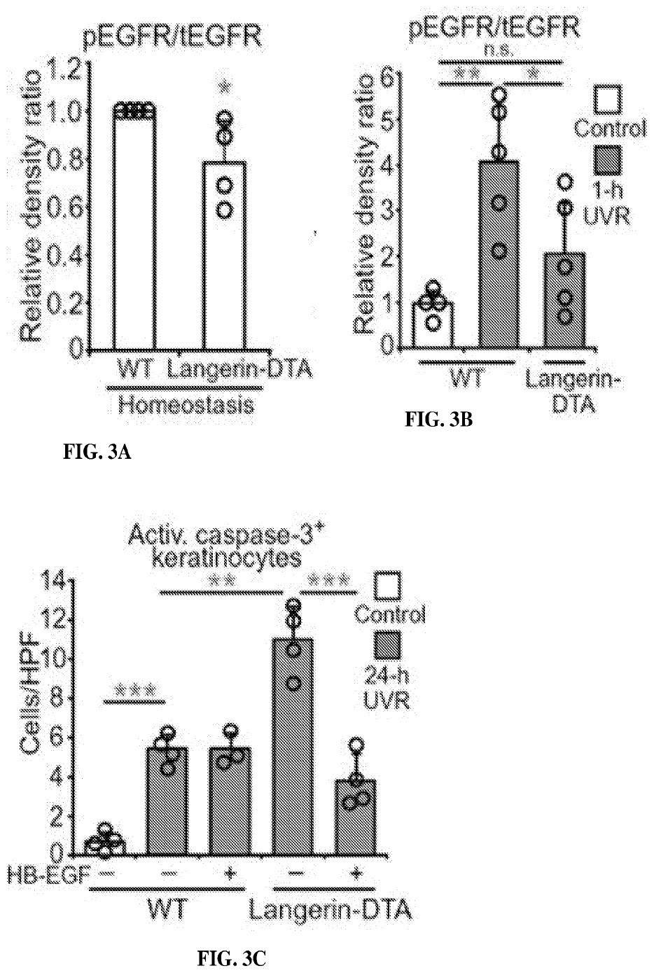

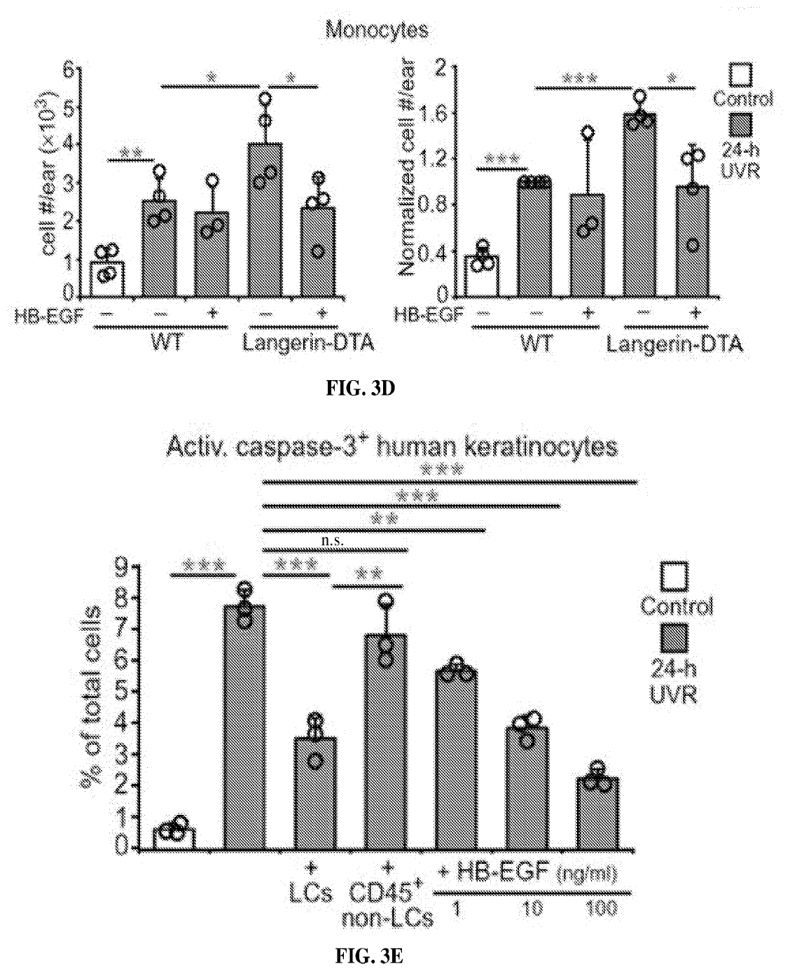

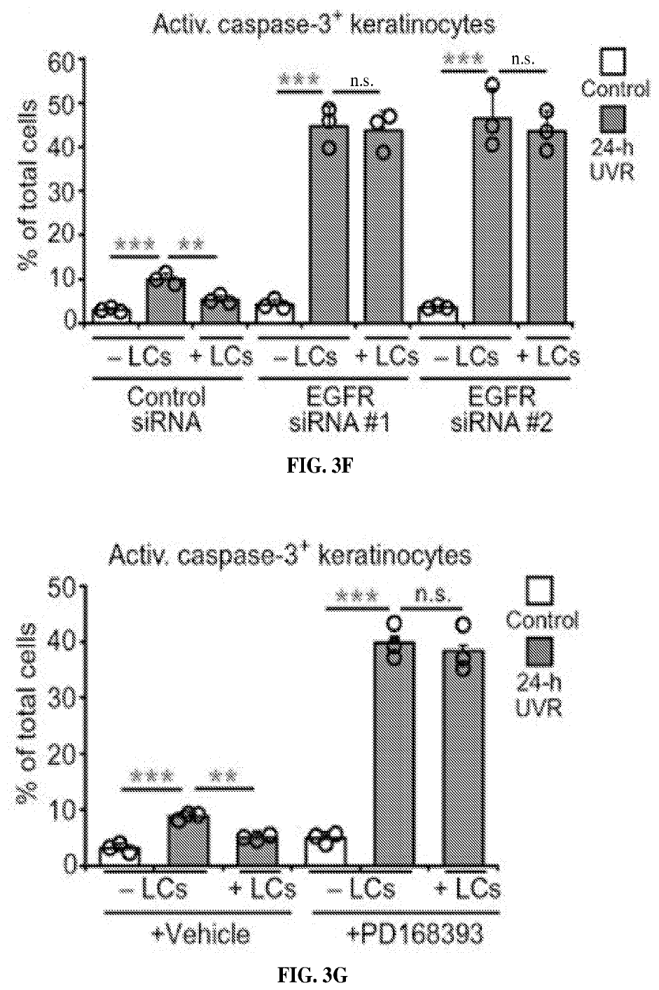

We then examined the effects of LC absence on UVR-induced keratinocyte EGFR activation. Epidermal EGFR showed increased phosphorylation by 1 hour after UVR exposure (FIG. 12F) (21), so we assessed this time point in subsequent experiments. The epidermis from Langerin-DTA mice had a modest reduction in homeostatic EGFR phosphorylation (FIG. 3A) and phosphorylation was not upregulated after UVR (FIG. 3B). These results suggested that LCs mediated the UVR-induced keratinocyte EGFR activation.

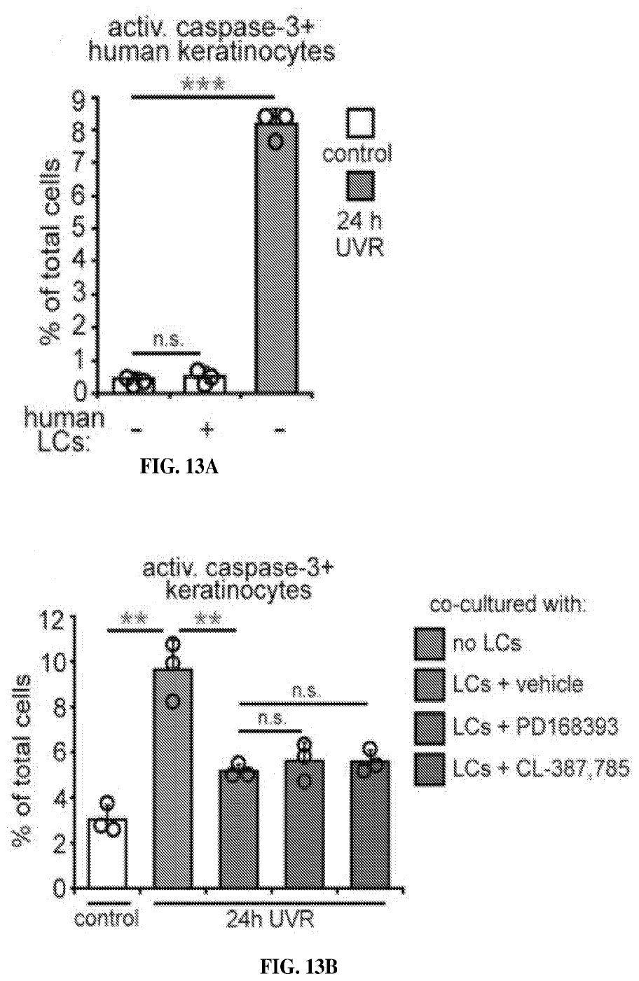

We asked if the LC-dependent EGFR stimulation was protective for keratinocytes. Treatment of Langerin-DTA mice with HB-EGF, a potent EGFR ligand (30), reduced UVR-induced apoptotic keratinocyte and monocyte accumulation (FIGS. 3C,3D). In vitro, adding human LCs or HB-EGF to keratinocytes were similar in limiting UVR-induced apoptosis (FIGS. 3E, and 13A). Furthermore, siRNA-mediated knockdown of Egfr (not shown) or EGFR activation in keratinocytes (not shown) abolished the protective effect of LCs (FIGS. 3F and 3G) while EGFR activation in LCs did not (FIG. 13B). Together, these results suggested that LCs limit UVR-induced keratinocyte apoptosis and skin inflammation by stimulating keratinocyte EGFR.

### EXAMPLE 5: LC ADAM17 IS CRITICAL FOR LIMITING PHOTOSENSITIVITY AND IS ACTIVATED BY UVR

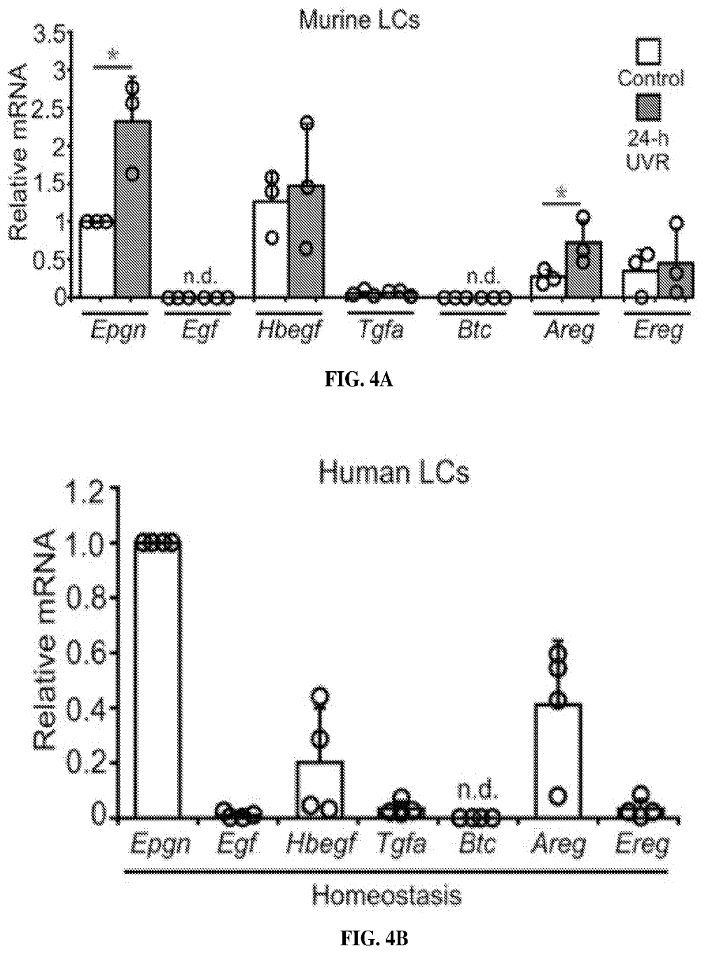

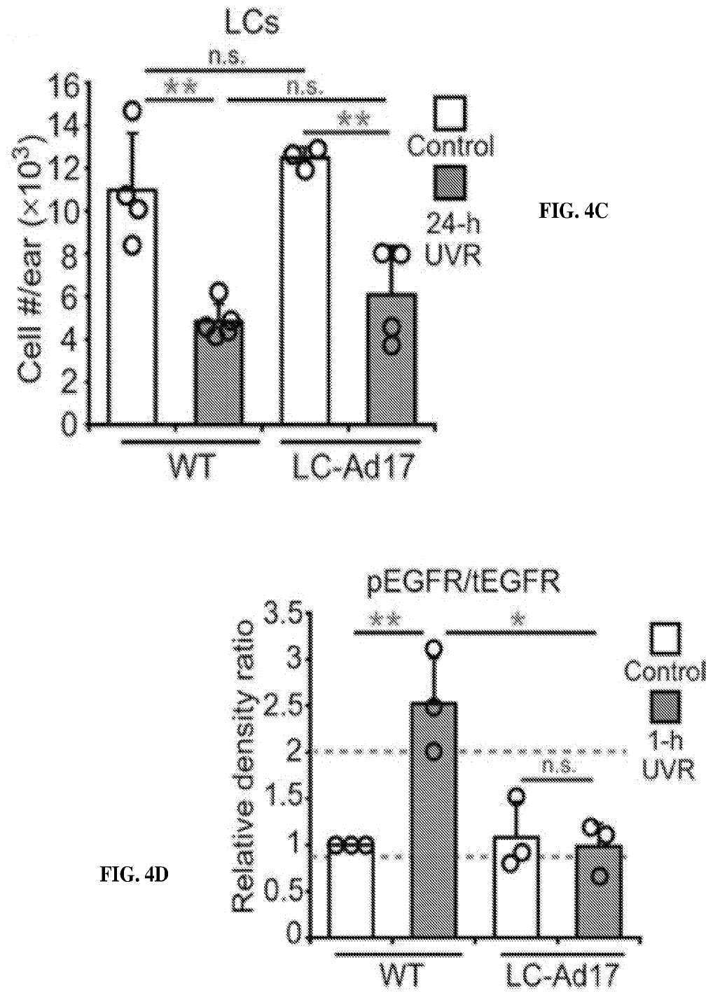

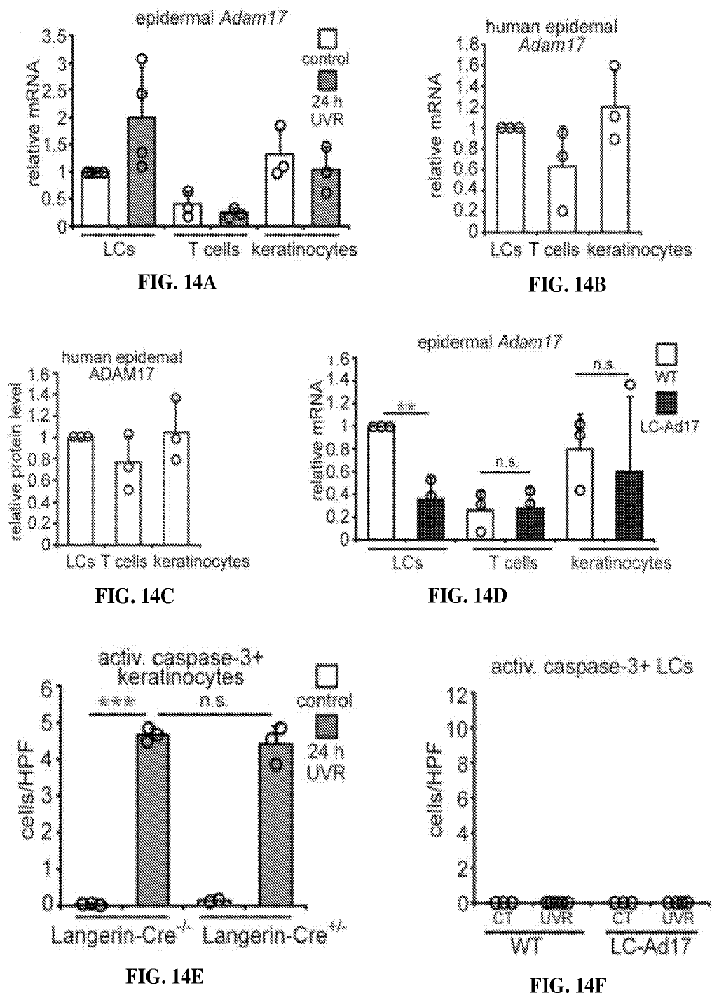

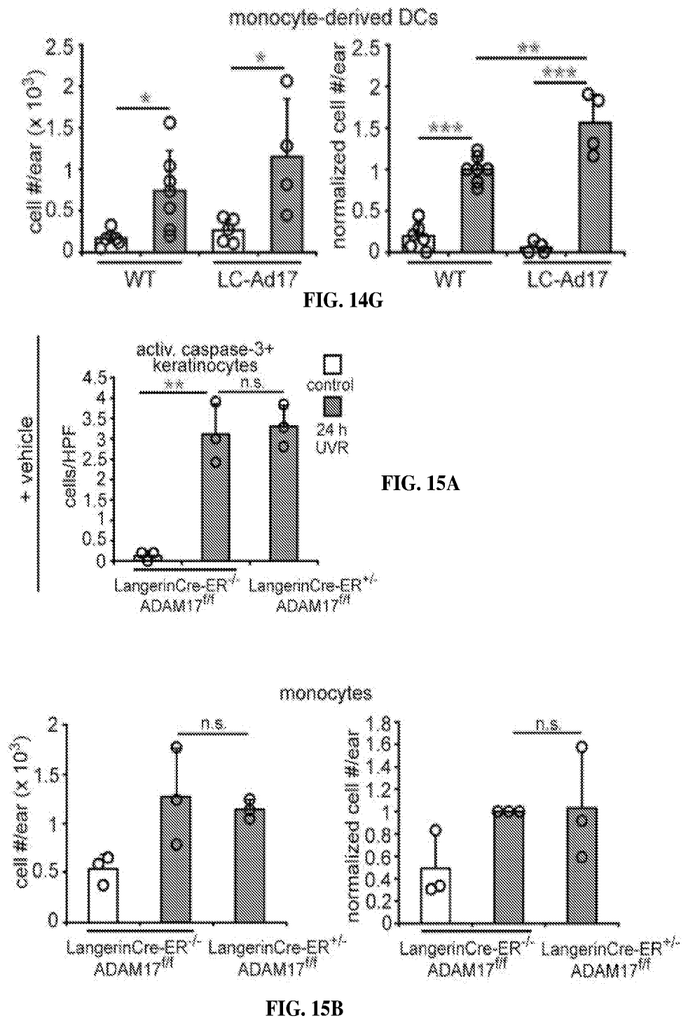

We asked whether LCs could be a key source of EGFR ligands. Both murine and human LCs expressed multiple EGFR ligands, such as epigen and amphiregulin, which were upregulated by UVR exposure in murine LCs (FIGS. 4A, 4B). A disintegrin and metalloprotease 17 (ADAM17) is a membrane-associated metalloprotease that is necessary for the cleavage and activation in cis of all EGFR ligands except EGF and β-cellulin (31), coincidentally, the 2 ligands not expressed or minimally expressed by LCs (FIGS. 4A and 4B). Murine and human LCs expressed ADAM 17 (FIGS. 14A-14C). The expression of both EGFR ligands and ADAM17 supported the idea that LCs were potentially capable of directly activating keratinocyte EGFR.

As LCs expressed multiple EGFR ligands, we assessed the role of LC-derived EGFR ligands by crossing ADAM17flox/flox mice (32) with Langerin-Cre+/− mice (33) to generate Langerin-Cre+/−ADAM17flox/flox mice (LC-Ad17 mice) that have Adam17 constitutively deleted from LCs (FIG. 14D). The Langerin-Cre driver itself had no effect on UVR-induced keratinocyte apoptosis (FIG. 14E), so experiments henceforth used Langerin-Cre−/−ADAM17flox/flox mice as controls (WT). Although WT and LC-Ad17 mice had comparable LC numbers (FIG. 4C), LC-Ad17 mice showed reduced UVR-induced EGFR phosphorylation (FIG. 4D), suggesting that LC-derived ADAM17 was important for UVR-induced keratinocyte EGFR activation.

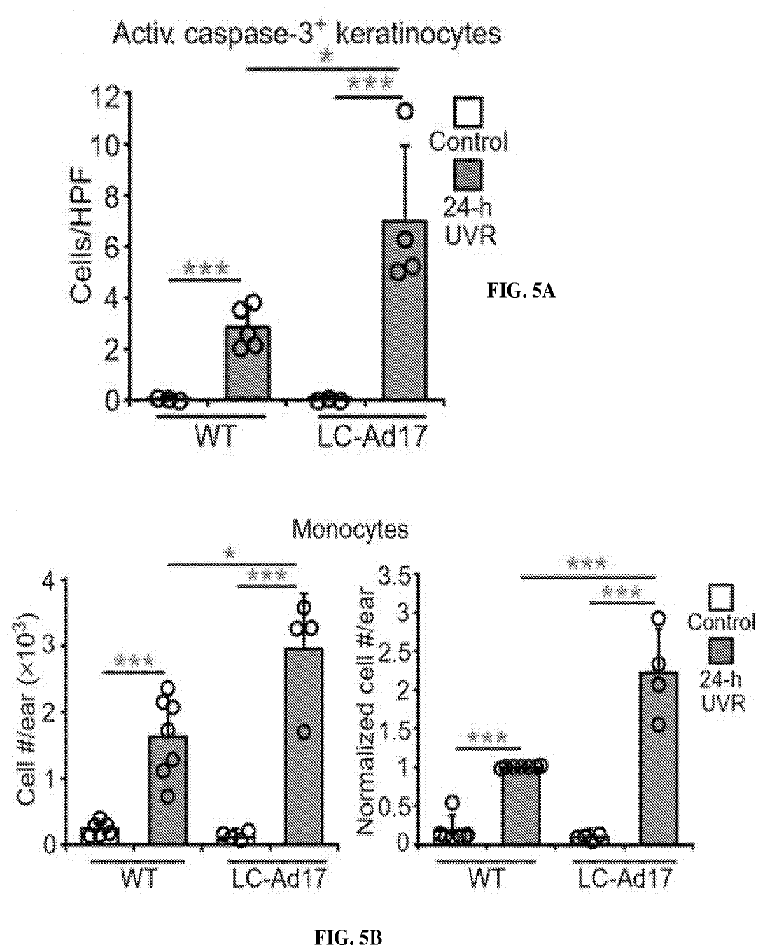

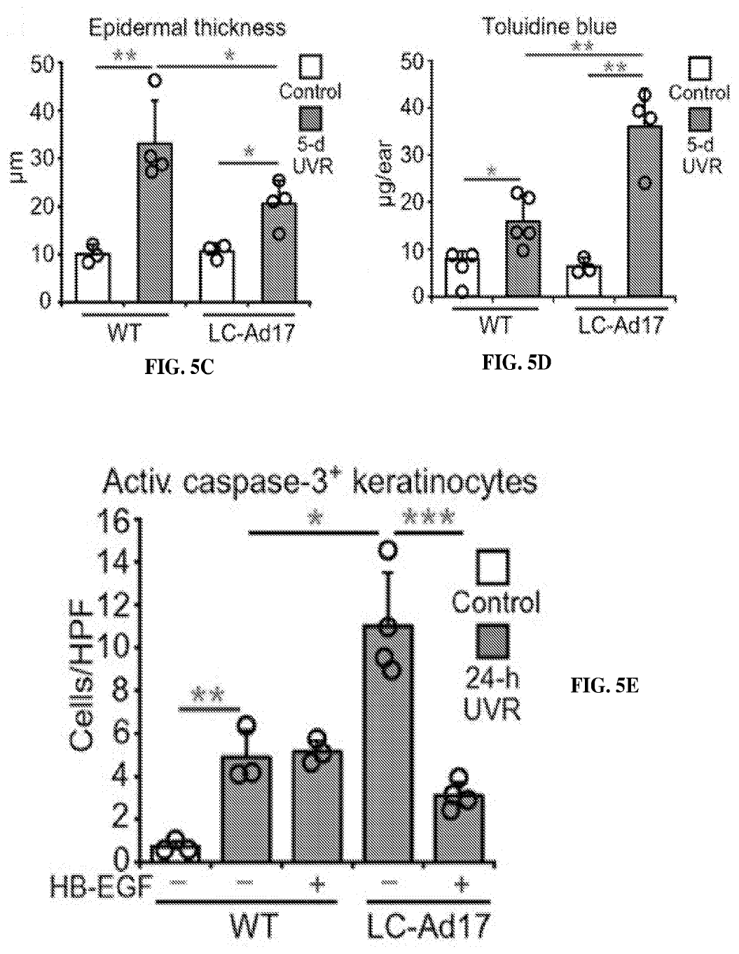

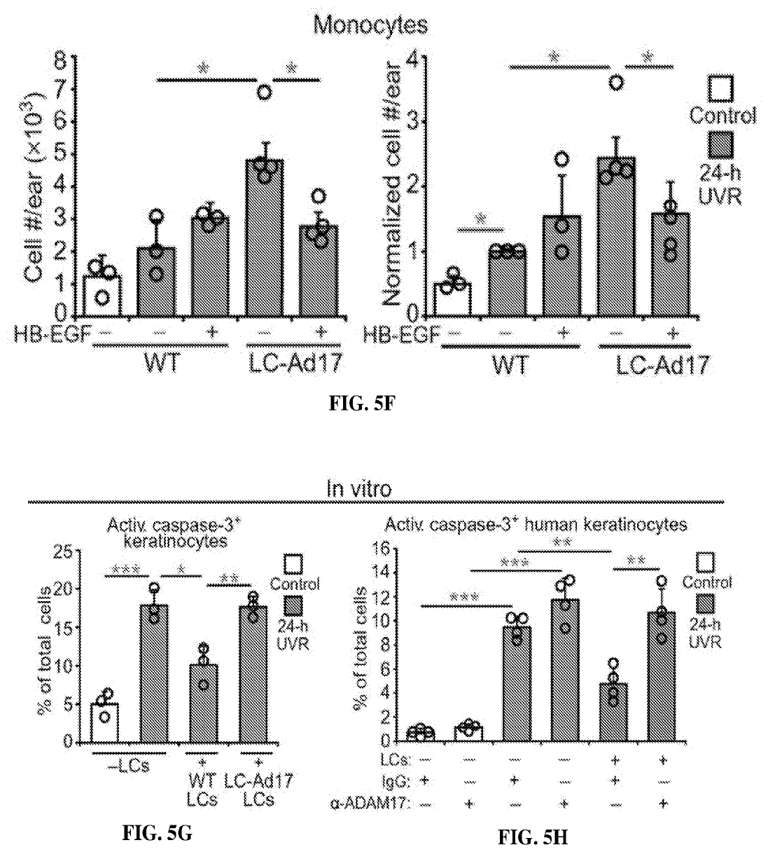

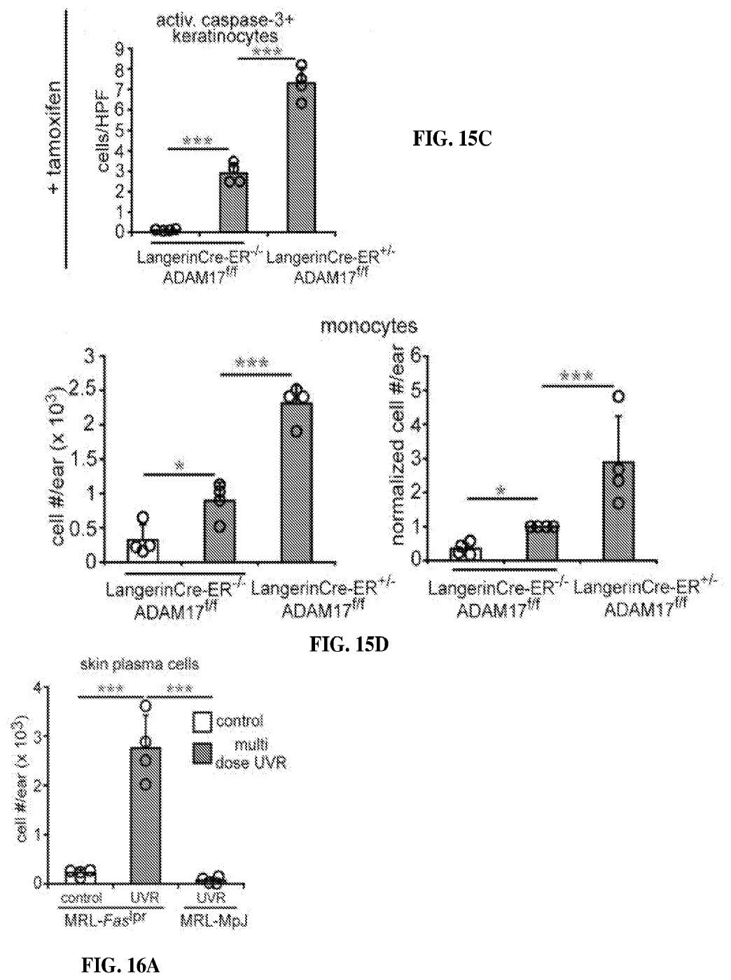

We further asked about the importance of LC ADAM17 in protecting skin. The LC-Ad17 mice showed increased accumulation of apoptotic keratinocytes, monocytes, and monocyte-derived DCs (FIGS. 5A, 5B, 14F, and 14G), blunted epidermal hyperplasia (FIG. 5C), and increased epidermal permeability (FIG. 5D) after UVR exposure. Inducible deletion in LCs (34) of ADAM17 in Langerin-Cre-ER+/−ADAM17flox/flox mice also increased UVR-induced keratinocyte apoptosis and monocyte accumulation (FIG. 15D). HB-EGF treatment dampened the increased UVR-induced keratinocyte apoptosis and skin inflammation in LC-Ad17 mice (FIGS. 5E, 5F), supporting the idea that the effect of LC ADAM17 deletion involved EGFR signals. In vitro, ADAM17-deficiency or blockade rendered LCs unable to protect keratinocytes from UVR-induced apoptosis in both murine and human systems (FIGS. 5G, 5H). These results together strongly supported the idea that LCs limit UVR effects via ADAM17 and stimulating keratinocyte EGFR.

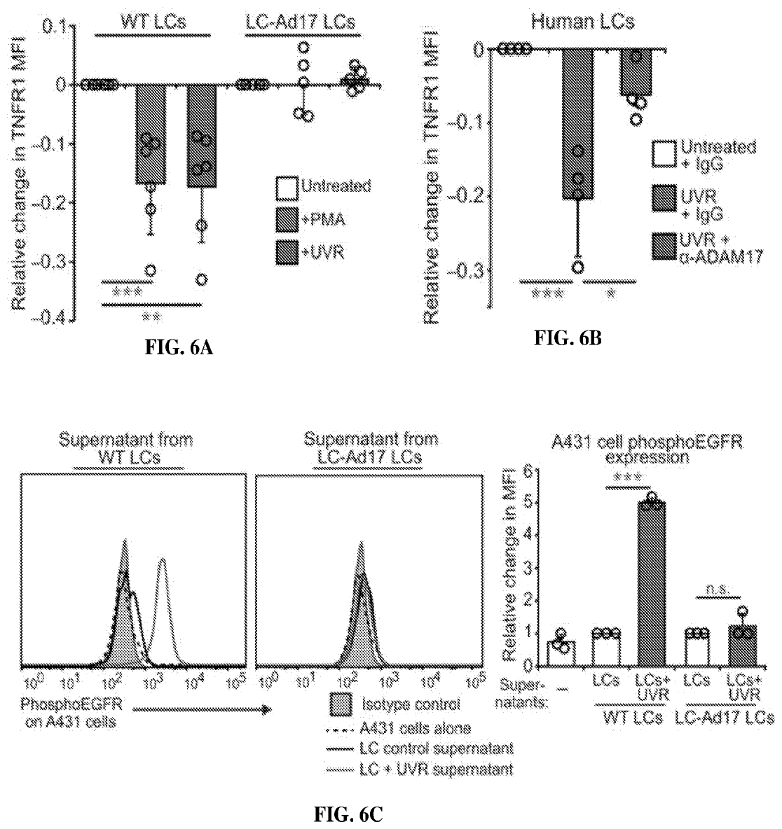

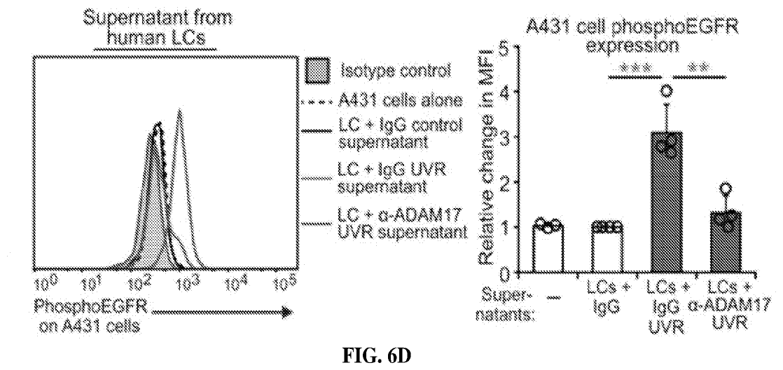

The rapid LC-dependent increase in epidermal EGFR activation with UVR suggested that LC ADAM17 could be activated by UVR. To measure ADAM17 activity, we quantified the level of cell-surface tumor necrosis factor receptor 1 (TNFR1), a substrate for ADAM17 (31). Treatment with PMA, a known ADAM17 activator (31), reduced murine LC TNFR1 in an ADAM17-dependent manner, as expected (FIG. 6A). Similar to PMA, UVR rapidly reduced TNFR1 on both murine and human LCs (FIGS. 6A,6B). This effect was abrogated by Adam17 deletion or ADAM17 blockade (FIGS. 6A, 6B). These results suggested that ADAM17 on LCs can be rapidly activated by UVR.

To examine whether the UVR-induced ADAM17 activation actually resulted in EGFR ligand cleavage and release, we collected conditioned supernatants from UVR-exposed LCs and assessed how well the supernatants induced EGFR phosphorylation in EGFR-overexpressing A431 indicator cells (figs S8 of provisional application; not shown). The validation of EGFR ligand release assay and characterization of keratinocyte EGFR ligand release involve the following: A431 indicator cells were serum-starved overnight then pre-treated for 15 minutes with vehicle, the irreversible EGFR activators PD168393 (2 μM), or CL-387,785 (1 μM). The cells were then treated with EGF (100 ng/mL) for 10 minutes and phosphoEGFR was measured by flow cytometry (n=2 separate cell passages). The EGFR ligand release assay used A431 indicator cells. Cells (sorted murine LCs, sorted human LCs, or primary murine keratinocytes) were treated or not with UVR and the conditioned supernatant was collected and added to serum-starved A431 cells for 10 minutes. The A431 cells were then collected and phosphoEGFR was measured by flow cytometry as an indicator of the level of EGFR ligand in the conditioned supernatant. Characterization of murine primary keratinocyte EGFR ligand release was based on exposing confluent primary murine keratinocytes to UVR. The supernatant of these cells and non-exposed control keratinocytes was collected 45 minutes later and added to A431 cells as above described in FIG. 15A. In contrast to supernatants from murine or human LCs that were not exposed to UVR, supernatants from UVR-exposed LCs induced a robust increase in A431 cell EGFR phosphorylation and Adam17 deletion or ADAM17 blockade abolished this effect (FIGS. 6C, 6D). UVR has been shown to activate ADAM17 on keratinocytes (35), and UVR exposure also caused murine keratinocytes to release more EGFR ligands, although this effect was less pronounced than that seen in the LCs (figs not shown). These data further established that UVR can trigger ADAM17 activation on LCs and showed that this activation can result in greater availability of active EGFR ligand. Together, our results show a central role for LCs and LC-derived ADAM17 in vivo and UVR-induced ADAM17 activation on LCs ex vivo, suggesting that there is an LC-keratinocyte axis whereby UVR induces LC ADAM17 activation and consequent EGFR ligand cleavage, leading to increased keratinocyte EGFR activation, which limits UVR-induced keratinocyte apoptosis and skin injury.

### EXAMPLE 6: THE LC-KERATINOCYTE AXIS IS DYSFUNCTIONAL IN PHOTOSENSITIVE SLE MODELS AND HUMAN SLE

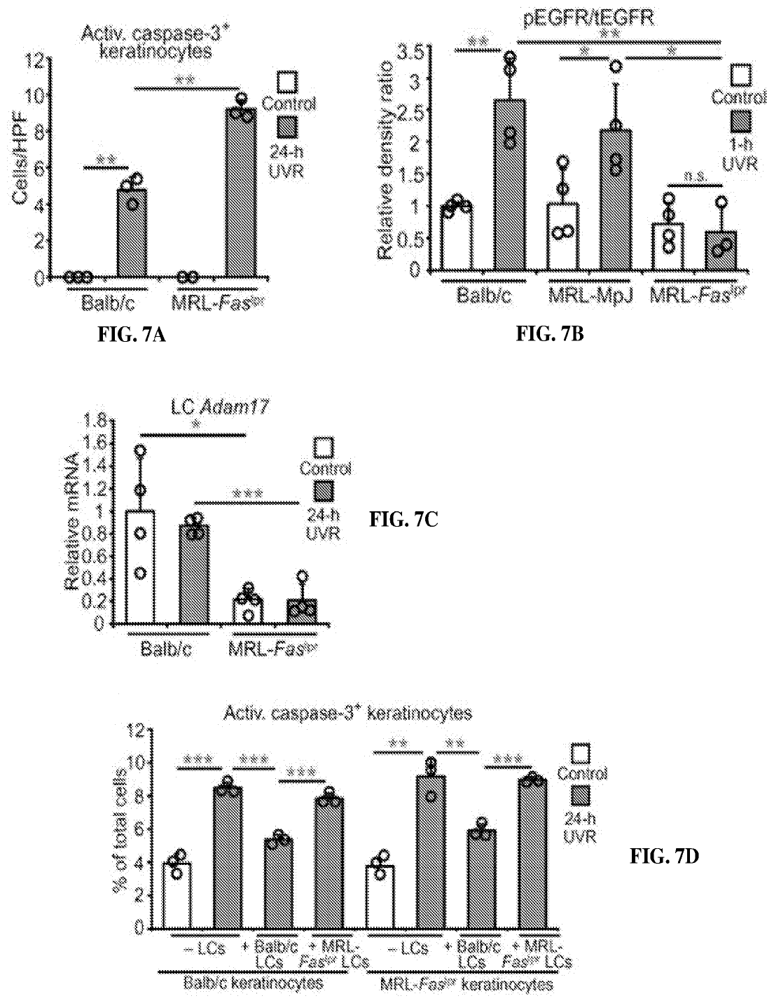

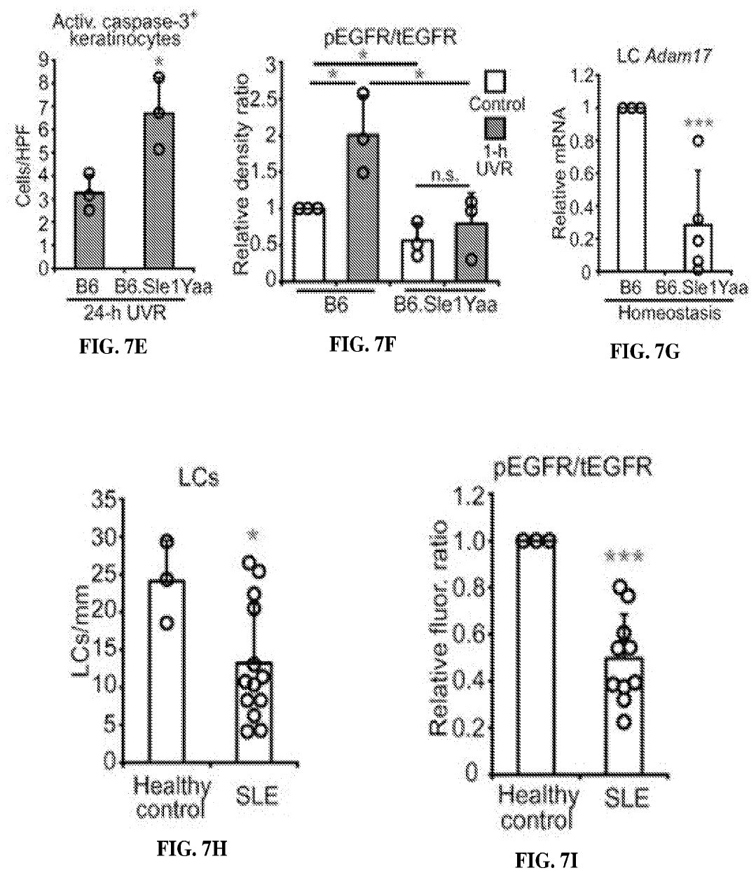

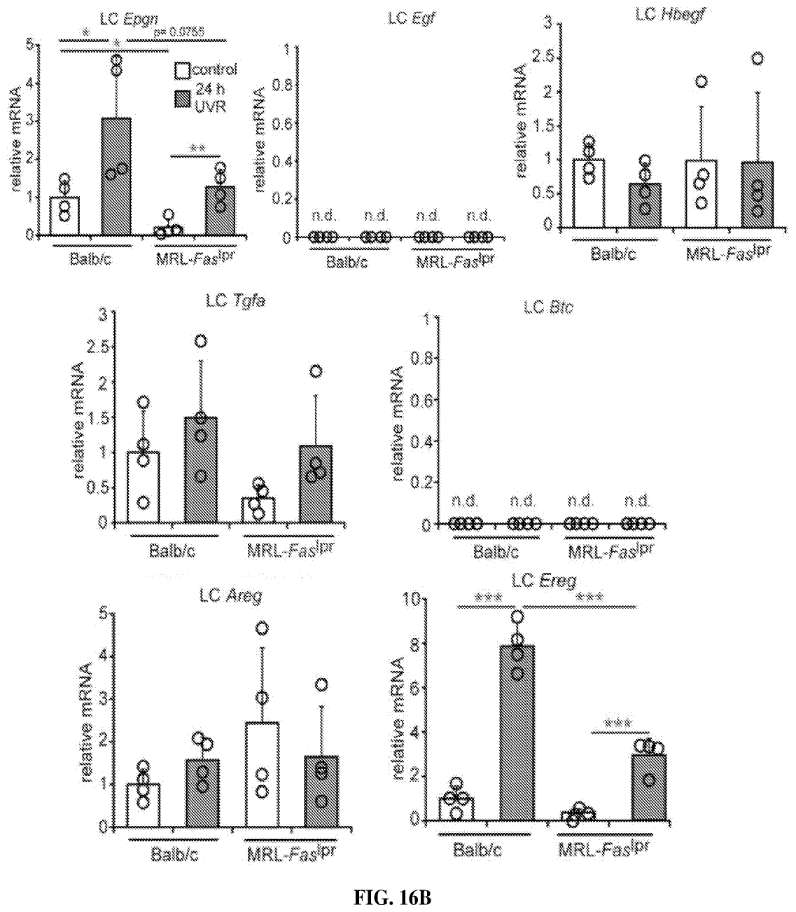

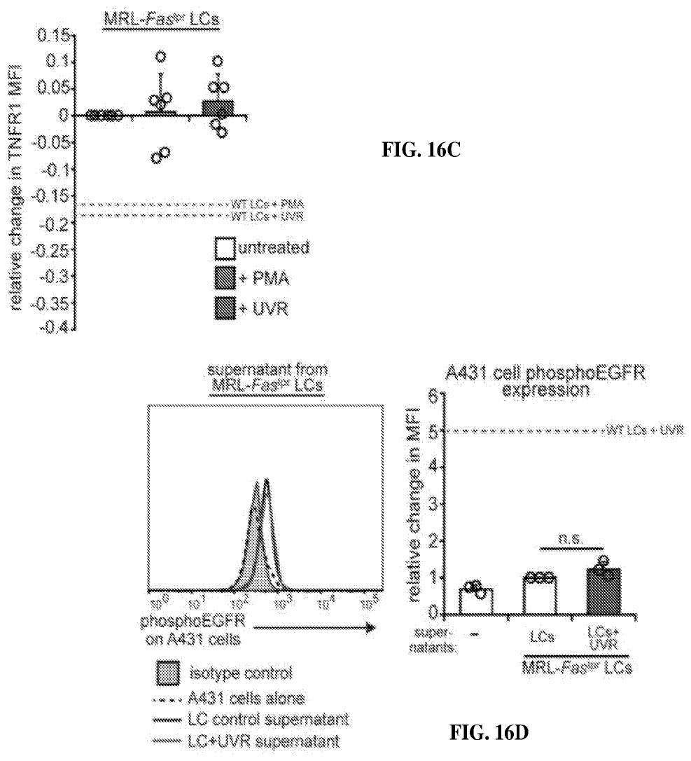

We asked whether photosensitivity in SLE models at least in part reflected dysfunction of this LC-keratinocyte axis. The MRL-Faslpr SLE model is a known photosensitive strain, developing more UVR-induced skin pathology than control Balb/c and/or MRL-MpJ mice (36, 37). UVR induces increased apoptotic keratinocyte accumulation in MRL-Faslpr mice (FIG. 7A) (36) along with skin plasma cell accumulation (FIG. 16A). Consistent with the possibility of a dysfunctional LC-keratinocyte axis, MRL-Faslpr mice showed reduced UVR-induced epidermal EGFR phosphorylation (FIG. 7B).

LC numbers are comparable between MRL-Faslpr mice and Balb/c controls (38) and we asked about their ability to protect skin. MRL-Faslpr LCs showed a trend toward reduced expression of epigen, the most abundantly expressed EGFR ligand, with UVR, and reduced expression of epiregulin (FIG. 16B), a ligand with relatively low expression (FIG. 4A). Adam17 mRNA, on the other hand, was reduced in MRL-Faslpr mice by about 70% at homeostasis and after UVR exposure (FIG. 7C). Consistent with the reduced Adam17 expression, MRL-Faslpr LCs showed no UVR-induced ADAM17 activation as indicated by TNFR1 changes or release of EGFR ligands (FIGS. 16C-D). In vitro, MRL-Faslpr LCs did not limit UVR-induced keratinocyte apoptosis (FIG. 7D) while control LCs could limit UVR-induced apoptosis of MRL-Faslpr keratinocytes (FIG. 7D), suggesting that LC dysfunction was the critical defect leading to increased UVR sensitivity in MRL-Faslpr mice. These data together suggested that MRL-Faslpr LCs, because of reduced ADAM17 and potentially because of reduced EGFR ligand expression, were unable stimulate epidermal EGFR, thus contributing to photosensitivity.

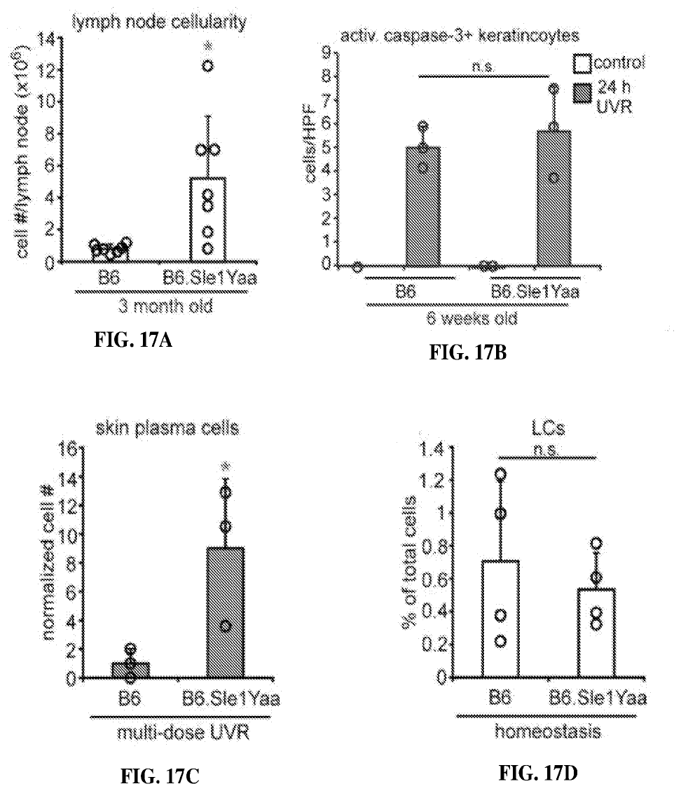

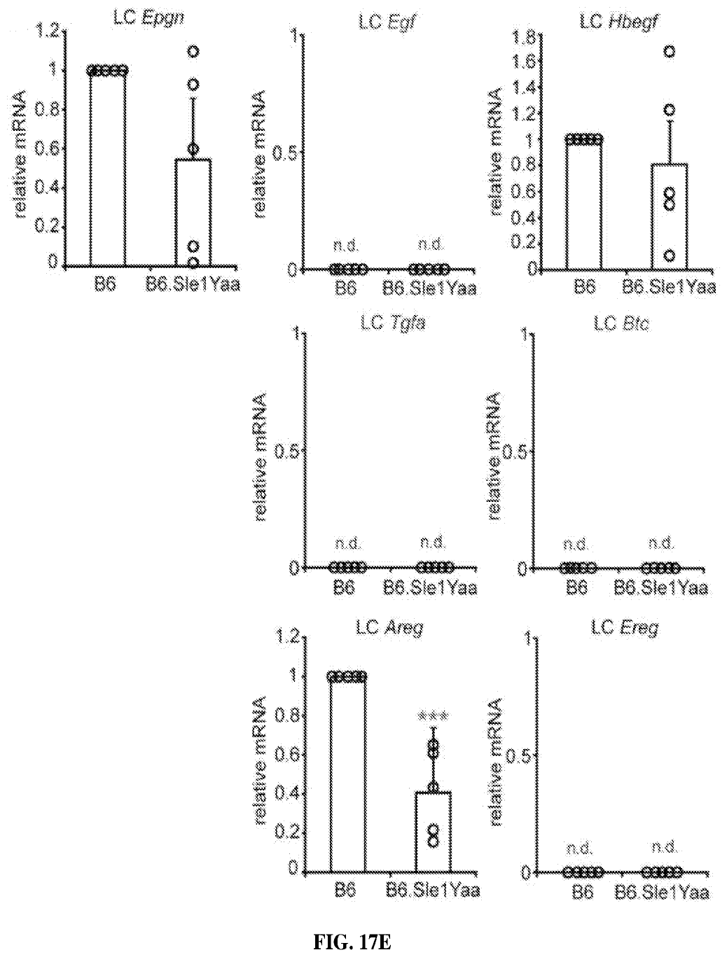

We also examined the B6. Sle1yaa model of SLE. These mice carry the Sle1 lupus susceptibility locus derived from lupus-prone NZB2410 mice along with the Y chromosome autoimmune accelerator locus whose activity is attributable to TLR7 duplication (39). The mice develop lymphadenopathy by 3 months (FIG. 17A), splenomegaly and autoantibody production by 4 months, and nephritis by 12 months (40). However, the photosensitivity of this model is unknown. Six week old B6. Sle1yaa mice did not show increased UVR-induced keratinocyte apoptosis (Fig. S10B), but 8-12 month old B6.Sle1yaa mice did (FIG. 7E). Upon multi-day UVR treatment, 8-12 month old B6.Sle1yaa mice developed skin lesions as early as 2 days while B6 mice did not (not shown). The skin findings were associated with the presence of plasma cells in the skin (FIG. 17C). These results indicated that diseased B6.Sle1yaa mice are photosensitive.

The 8-12 month old B6.Sle1yaa mice also showed reduced UVR-induced epidermal EGFR activation relative to controls (FIG. 7F). LC numbers were unchanged and only the EGFR ligand amphiregulin was reduced (FIGS. 17D, 17E), but B6.Sle1yaa LCs showed reduced Adam17 mRNA expression (FIG. 7G). These data together suggested that photosensitivity in both SLE models may be attributable at least in part to a dysfunctional LC-keratinocyte axis whereby LCs are less able to produce activated EGFR ligands to stimulate keratinocyte EGFR.

We examined human SLE skin for signs of a dysfunctional LC-keratinocyte axis. Non-sun-exposed, nonlesional SLE skin showed decreased LC numbers relative to healthy control skin (FIG. 7H), suggesting an abnormality in LC function and a potential for reduced input of EGFR ligands. Epidermal EGFR phosphorylation was also reduced in SLE skin (FIG. 7I). These data support the idea that the LC-keratinocyte axis is dysfunctional in human SLE.

### EXAMPLE 7: TOPICAL EGFR LIGAND REDUCES PHOTOSENSITIVITY IN AN SLE MODEL

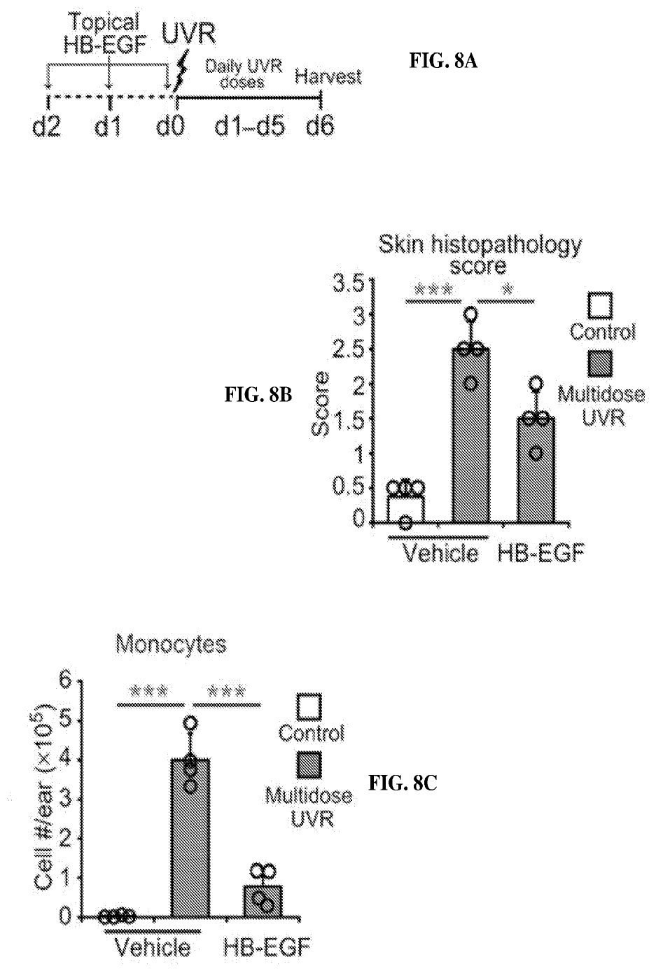

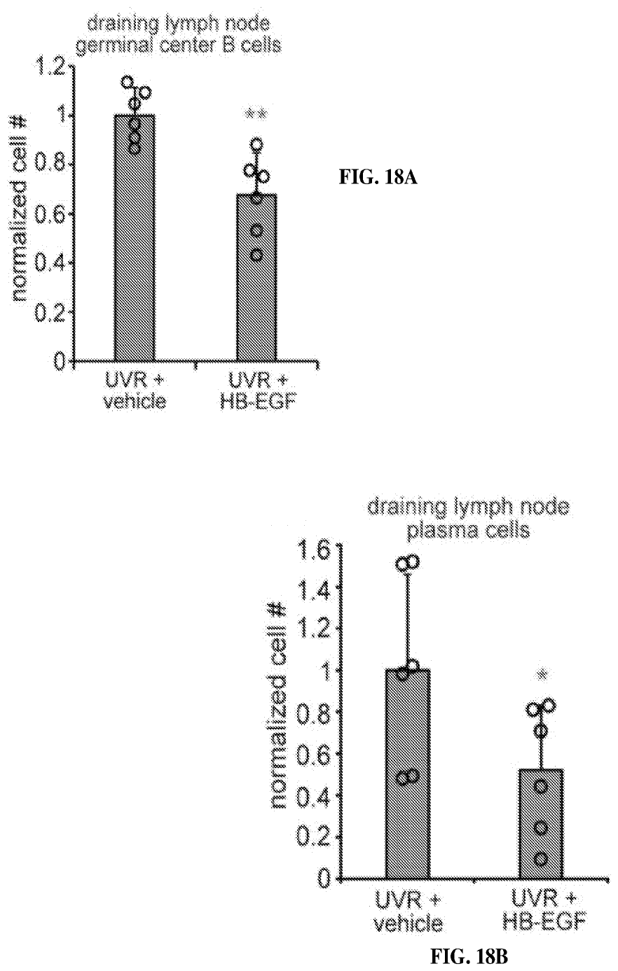

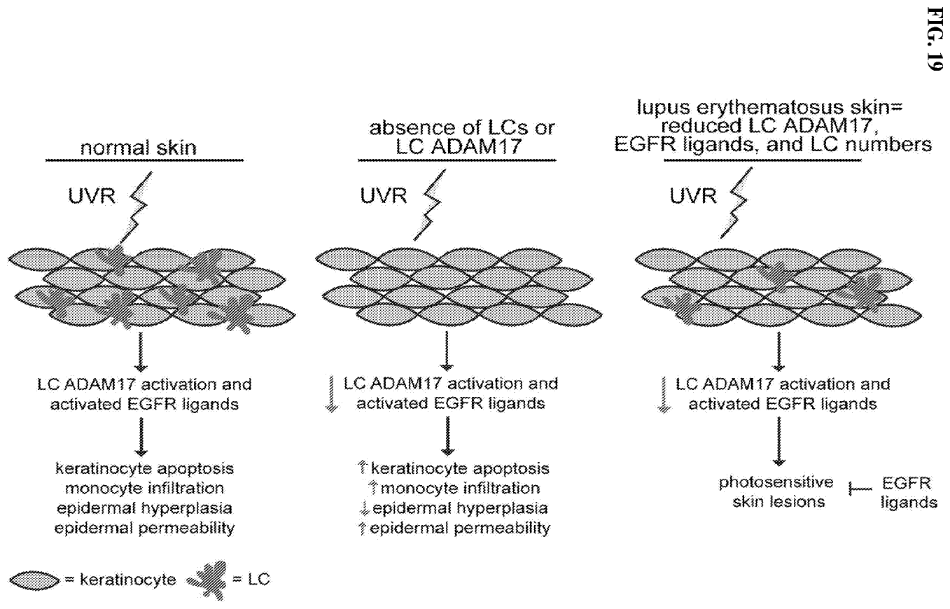

We asked whether EGFR ligand supplementation could reduce photosensitivity in MRL-Faslpr mice. Multi-day UVR exposure has been shown to increase complement and immunoglobulin deposition in skin (36) and we observed that this regimen also led to ulcerations with a neutrophil-dominant infiltrate (FIGS. 8A-8C). Topical treatment with HB-EGF (FIG. 8A) reduced the severity of UVR-induced skin lesions (FIGS. 8B) and monocyte accumulation (FIG. 8C). Topical HB-EGF also reduced germinal center B cells (FIG. 18A) and plasma cells (FIG. 18B) in skin-draining lymph nodes, suggesting that modulating skin EGFR signaling may impact systemic immunity. These findings suggest that compensating for a dysfunctional LC-keratinocyte axis by providing EGFR ligand can be used as an approach to treating photosensitivity.

### EXAMPLE 8: ADDITIONAL MATERIALS AND METHODS

### A. Mice

Mice from 6-12 weeks old were used unless otherwise stated and were sex and aged-matched. Both male and female mice were used for experiments, except for B6. Sle1yaa mice, in which only males were used because the model is dependent in part on the autoimmune accelerator locus on the Y chromosome (40). C57BL/6J, Langerin-DTA, Rag1−/−, Balb/c, MRL-MpJ, MRL-Faslpr, and B6. Sle1yaa mice were originally from Jackson Laboratory (JAX) and bred at our facility. CCR2-GFP and CCR2-DTR mice (25) were bred at our facility. Rag1−/− mice were intercrossed with Langerin-DTA mice to generate Rag1−/− Langerin-DTA mice. ADAM17flox/flox mice (32) were intercrossed with Langerin-Cre+/− mice (33, 34) (National Cancer Institute (NCI)), and Langerin-CreER+/− YFP mice (34) to generate LC-Ad17 and Langerin-CreER+/−ADAM17flox/flox mice, respectively. The WT mice used in experiments involving LC-Ad17 mice were Langerin-Cre−/−ADAM17flox/flox littermate controls. All animal procedures were performed in accordance with the regulations of the Institutional Animal Use and Care Committee at the Hospital for Special Surgery and Weill Cornell Medicine.

### B. Human Research Participants

For immunofluorescence analysis, non-sun-exposed nonlesional skin from the buttocks of healthy controls and SLE patients was used. With the exception of one healthy control, all skin samples were from samples examined in (53). Controls were between the ages of 28-65 and 67% were female. The SLE patients met American College of Rheumatology criteria for SLE, were between the ages of 19-62 years old, and 79% were female. All SLE patients were currently receiving treatment at the time of the biopsy (53). These samples were collected and used in accordance with the Institutional Review Board at the NYU School of Medicine (IRB# S14-00487).

For human LC and epidermal CD45+ non-LC cell isolation, human skin samples were collected from eleven human patients undergoing elective reconstructive surgery at the Division of Plastic and Reconstructive Surgery at the Memorial Sloan Kettering Cancer Center (MSKCC). Ten of the eleven patients were female and the patients were between the ages of 41-69 at the time of surgery. All tissue collection and research use adhered to protocols approved by the Institutional Review and Privacy Board at the Memorial Sloan Kettering Cancer Center, and all participants signed written informed consents (IRB#06-107).

### C. Mouse Treatments

For indicated 24 hour experiments, HB-EGF (2 ug; R&D Systems) dissolved in dimethyl sulfoxide (DMSO) was applied to each ear 15 minutes prior to UVR exposure. For long-term lesion development experiments, mice were shaved in a small area on the lower back. At 24 hours, HB-EGF was applied on the ears as above and on the shaved back area (8 ug) for three consecutive days. Mice received their first dose of UVR on the last day of HB-EGF treatment.

### D. Flow Cytometry, Cell Sorting, and Quantification

For staining of murine whole skin, single cell suspensions of skin were generated as previously described (14). Briefly, ear skin was excised, finely minced, digested in collagenase type II (616 U/mL; Worthington Biochemical Corporation), dispase (2.42 U/mL; Life Technologies), and DNAsel (80 μg/mL; Sigma-Aldrich), incubated at 37° C. while shaking at 100 rpm, triturated with glass pipettes, and filtered. For murine epidermal cell staining or sorting, ear and trunk skin was incubated in dispase at 37° C. for 45 minutes. The epidermis was then scraped off and finely minced before digestion in collagenase type II.

For flow cytometry analysis, the following gating strategies were used after excluding debris and non-single cells: LCs: Lineage (CD3, B220, NK, Ly6G)-, CD45+ CD11b+ CD24+, CD11c+, MHCII+; monocytes: Lineage-, CD45+, CD11b+, CD24-, Ly6C+, MHCII-; monocyte-derived DCs: Lineage-CD45+ CD11b+ CD24-Ly6Chi-lo, MHCII+; CD11b+ DCs: Lineage-CD45+ CD24-CD11b+ Ly6C-CD64-CD11c+ MHCII+; CD11b− DCs: Lineage-CD45+ CD11b− CD24+ CD11c+ MHCII+; macrophages: Lineage-CD45+ CD11b+ CD24-CD64+; neutrophils: Lineage+ CD11b+ Ly6Cmed, side scatter (SSC)hi; T cells: epidermal CD45+, CD11b−, CD3+; keratinocytes: epidermal CD45−, CD31−, EpCAM+ or total skin CD45−, CD31−, CD49f+, Sca1+, EpCAM+; skin plasma cells: CD45+, B220lo, CD3−, intracellular IgGhi; lymph node germinal center B cells: CD3−, B220+, PNA+; lymph node plasma cells: CD3−, B220lo, CD138+. LCs, monocytes, monocyte-derived DCs, CD11b+ DCs, CD11b− DCs, and macrophages were gated according to Tamoutounour et al. (24). Primary and secondary antibodies are described in Table S1 and Table S2.

TABLE S1
List of primary antibodies.
FC = Flow Cytometry, IF = Immunofluorescence,
WB = Western Blot, FB = Functional Blocking

| ANTIBODY (CLONE) | SUPPLIER | CATALOG # | LOT # | APPLN |
|---|---|---|---|---|
| Armenian hamster anti-mouse | BioLegend | 100304 | B216147 | FC, IF |
| CD3 biotin (145-2C11) | | | | |
| rat anti-mouse Ly6G biotin | BioLegend | 127604 | B218529 | FC |
| (1A8) | | | | |
| rat anti-mouse B220 biotin | BioLegend | 103204 | B191786 | FC |
| (RA3-6B2) | | | | |
| rat anti-mouse CD49b biotin | eBiosciences | 13-5971-81 | 4295252 | FC |
| (DX5) | | | | |
| rat anti-mouse CD45 | BioLegend | 103128 | B211311 | FC |
| AlexaFlour700 (30-F11) | | | | |
| rat anti-mouse CD45 | BioLegend | 103132 | B218549 | FC |
| PerCPCy5.5 (30-F11) | | | | |
| Armenian hamster CD11c | BioLegend | 117324 | B237079 | FC |
| APCCy7 (N418) | | | | |
| mouse anti-mouse Iab PE | BioLegend | 116408 | B177711 | FC |
| (AF6-120.1) | | | | |
| mouse anti-mouse Iab FITC | BDBiosciences | 553551 | 62094 | FC |
| (AF6-120.1) | | | | |
| rat anti-mouse CD24 | BioLegend | 101824 | B216147 | FC |
| PerCPCy5.5 | | | | |
| rat anti-mouse CD11b | BioLegend | 101233 | B236974 | FC |
| Brillant Violet 570 (M1/70) | | | | |
| rat anti-mouse CD11b PE | BioLegend | 101208 | B228654 | FC |
| (M1/70) | | | | |
| rat anti-mouse CD11b FITC | BioLegend | 101206 | B192968 | FC |
| (M1/70) | | | | |
| rat anti-mouse CD3 APCCy7 | BioLegend | 100330 | B190252 | FC |
| (145-2C11) | | | | |
| rat anti-mouse CD3 FITC | BDBiosciences | 553062 | 5166876 | FC |
| (145-2C11) | | | | |
| rat anti-mouse Ly6C PECy7 | BioLegend | 128018 | B242951 | FC |
| (HK1.4) | | | | |
| rat anti-mouse Ly6C FITC | BioLegend | 128006 | B180475 | FC |
| (HK1.4) | | | | |
| mouse anti-mouse CD64 APC | BioLegend | 139306 | B207411 | FC |
| (X54-5/7.1) | | | | |
| mouse anti-mouse CD64 PE | BioLegend | 139304 | B171679 | FC |
| (X54-5/7.1) | | | | |
| rat anti-mouse CD31 | BioLegend | 102420 | B219868 | FC |
| PerCPCy5.5 (390) | | | | |
| rat anti-mouse EpCAM PECy7 | BioLegend | 118216 | B176413 | FC |
| (G8.8) | | | | |
| rat anti-mouse EpCAM APC | BioLegend | 118214 | B173069 | FC |
| (G8.8) | | | | |
| rat anti-mouse Sca-1 APCCy7 | BioLegend | 108126 | B214144 | FC |
| (D7) | | | | |
| rat anti-mouse CD49f biotin | BioLegend | 313604 | B226568 | FC |
| (GoH3) | | | | |
| Armenian hamster anti-mouse | BioLegend | 121406 | B184715 | FC |
| CD103 PE (2E7) | | | | |
| Armenian hamster anti-mouse | BioLegend | 121413 | B222546 | FC |
| CD103 APC (2E7) | | | | |
| Armenian hamster TNFR1/p55 | BioLegend | 113005 | B240777 | FC |
| APC (55R-286) | | | | |
| rat anti-mouse IgG1 FITC | BDBiosciences | 553443 | 92966 | FC |
| (A85-1) | | | | |
| rat anti-mouse IgG2a/2b | BDBiosciences | 553399 | 4150540 | FC |
| FITC (R240) | | | | |
| rat anti-mouse IgG3 FITC | BDBiosciences | 553403 | 7027876 | FC |
| (R40-82) | | | | |
| rat anti-mouse CD138 APC | BioLegend | 142505 | B237677 | FC |
| (281-2) | | | | |
| peanut agglutinin (PNA) | Vector Labs | B-1075 | X1221 | FC |
| biotin | | | | |
| mouse anti-human CD1a | BioLegend | 30016 | B236344 | FC |
| AlexaFlour 647 (HI149) | | | | |
| mouse anti-human HLA-DR | BioLegend | 307606 | B183424 | FC |
| (L243) | | | | |
| mouse anti-human CD45 | eBiosciences | 45-0459-71 | E029129 | FC |
| PerCPCy5.5 (HI30) | | | | |
| goat anti-mouse, human | Santa Cruz | sc-22620 | D2216 | IF |
| Langerin (E-17) | Biotechnology | | | |
| rabbit anti-mouse, human | R&D Systems | AF835 | CF23415101 | IF |
| active caspase-3 (polyclonal) | | | | |
| rabbit anti-human phospho- | BioCare Med. | API300AA | 013117 | IF |
| EGFR Tyr1068 (EP774Y) | | | | |
| mouse anti-human EGFR (H11) | BioCare Med. | ACI063A | 060517 | IF |
| rabbit anti-mouse phospho- | Cell Signaling | 3777S | 13 | FC, WB |
| EGFR Tyr1068 (D7A5) | | | | |
| goat anti-mouse total EGFR | R&D Systems | AF1280 | HXO0216012 | FC, WB |
| (polyclonal) | | | | |
| rabbit anti-mouse hsp90 | Cell Signaling | 4874S | 3 | WB |
| (polyclonal) | | | | |
| goat IgG polyclonal isotype | R&D Systems | AB-108-C | ES4115041 | FC, IF |
| control | | | | |
| rat IgG2a isotype control | BDBiosciences | 553928 | 4324804 | FC |
| (R35-95) | | | | |
| rabbit monoclonal IgG | Cell Signaling | 3900S | 25 | IF |
| isotype control (DA1E) | | | | |
| mouse IgG isotype control | R&D Systems | MAB002 | 1X1207041 | IF |
| (#11711) | | | | |
| human monoclonal anti-human | Abcam | 215268 | GR3192882-1 | FB, FC |
| IgG1 ADAM17 (D1(A12)) | | | | |
| monoclonal human IgG1 isotype | Adipogen | AG-35B- | A26741504 | FB, FC |
| control | | 0006-C100 | | |
| mouse anti-human CD3 PECy7 | BioLegend | 300419 | B208514 | FC |
| (UCHT1) | | | | |
| mouse anti-human HLA-DR | BioLegend | 307617 | B246747 | FC |
| APC-Cy7 (L243) | | | | |
| mouse anti-human EpCAM APC | BioLegend | 324207 | B155666 | FC |
| (9C4) | | | | |
| mouse anti-human CD1a PE | Beckman | IM1942U | 11 | FC |
| (BL6) | Coulter | | | |

TABLE S2
Secondary antibodies and other staining reagents.
FC = Flow Cytometry, IF = Immunofluorescence, WB = Western Blot

| ANTIBODY (CLONE) | SUPPLIER | CATALOG # | LOT # | APPLN |
|---|---|---|---|---|
| donkey anti-goat Alexa Fluor | Jackson | 705-545-003 | 128611 | FC |
| 488 (polyclonal) | Immunoresearch | | | |
| donkey anti-rabbit Alexa | Jackson | 711-606-152 | 125599 | FC |
| Fluor 647 (polyclonal) | Immunoresearch | | | |
| Streptavidin Pacific Blue | ThermoFisher | S11222 | 1870540 | FC |
| | Scientific | | | |
| | (Invitrogen) | | | |
| Streptavidin APC | ThermoFisher | S868 | 1124091 | FC |
| | Scientific | | | |
| | (Invitrogen) | | | |
| Streptavidin Alexa Flour 488 | ThermoFisher | S11223 | 1851449 | FC, IF |
| | Scientific | | | |
| | (Invitrogen) | | | |
| donkey anti-mouse IgG biotin | Jackson | 715-066-151 | 124850 | IF |
| | Immunoresearch | | | |
| donkey anti-human IgG biotin | Jackson | 709-066-098 | 135590 | FC |
| | Immunoresearch | | | |
| donkey anti-rabbit rhodamine | Jackson | 711-295-152 | 130068 | IF |
| | Immunoresearch | | | |
| donkey anti-goat Alexa Flour | Jackson | 705-605-147 | 124186 | IF |
| 647 | Immunoresearch | | | |
| donkey anti-rabbit HRP | Jackson | 711-035-152 | 128838 | WB |
| | Immunoresearch | | | |
| donkey anti-goat HRP | Jackson | 705-035-003 | 130633 | WB |
| | Immunoresearch | | | |
| Human TruStain FcX (Fc | BioLegend | 422301 | B247180 | FC |
| Receptor Blocking Solution) | | | | |
| DAPI | ThermoFisher | D1306 | 1023584 | FC, IF |
| | Scientific | | | |
| | (Invitrogen) | | | |

For flow cytometry analysis, cells were analyzed using a FACSCanto (BD Biosciences) and FlowJo Software (Tree Star). Cells were sorted using a BD Influx.

To measure phosphoEGFR by flow cytometry, cells were serum- or EGF-starved, pretreated with 2 mM NaVO3 for 15 minutes, fixed with 4% paraformaldehyde for 15 minutes at room temperature, and then permeabilized with ice-cold methanol (90%) for 30 minutes on ice. The cells were then stained with anti-phosphoEGFR Tyr1068 (Cell Signaling) followed by anti-rabbit Alexa647 (Jackson Immunoresearch).

For isolation of murine epidermal cells for cultures and qPCR, epidermal single cell suspensions from ear and back skin were flow sorted for CD45+CD11b+ EpCAM+ CD3− LCs, CD45+CD3+CD11b− T cells, and CD45−, CD31−, EpCAM+ keratinocytes. Purity of sorted cells was >95%.

For human LC and CD45+ non-LC isolations, fresh skin samples were obtained from patients undergoing elective reconstructive surgery as described above. Skin samples were cut into small pieces and incubated for 30 min at 37° C. and 5% CO2 in prewarmed DMEM/F-12 (Stem Cell Technologies) with dispase II (1 IU/ml; Roche Diagnostics) to facilitate separation of the epidermis from the dermis. The epidermis was gently peeled away from the dermis and placed in RPMI 1640 supplemented with 10 mM HEPES, 1% penicillin/streptomycin (Media Lab, MSKCC), 50 mM L-glutamine (Cellgro), 50 μM β-mercaptoethanol (Gibco, Life Technologies), and 10% heat-inactivated pooled healthy human serum (Atlanta Biologicals). The epidermal sheets were then finely minced and digested with collagenase as described for mouse epidermis. LCs (CD45+ CD1a+ HLADR+), non-LC CD45+ cells (CD45+ CD1a− HLADR−), T cells (CD45+ CD1a− HLADR− CD3+), and keratinocytes (CD45− CD1a− CD3− EpCAM+) were sorted and sorted cells had a purity ≥95%.

Human epidermal cells used for flow cytometric analysis of ADAM17 were incubated with human TruStain FcX Fc receptor blocking solution (BioLegend), the cells were then stained with anti-human ADAM17 (Abcam) or human IgG1 isotype control (Adipogen), followed by anti-human IgG biotin (Jackson Immunoresearch) and streptavidin Alexa 488 (Thermo Fisher Scientific-Invitrogen). After excluding debris, dead cells, and non-single cells the following gating strategies were used to examine ADAM17 expression: LCs: CD45+ CD1a+ HLADR+ CD3−, T cells: CD45+ CD3+ CD1a− HLADR− and keratinocytes: CD45− CD1a− HLADR− EpCAM+.

Cells were counted using a Z1 Coulter Counter (Beckman Coulter). To calculate absolute cell numbers, the percentage of the total of a particular population was multiplied by the total cell count from the Coulter Counter. For figures showing normalized values, the control sample was set to 1, and the experimental samples were normalized relative to the control for that experiment. For experiments that contained more than one control sample, the mean was obtained for the control samples, and the individual control and experimental samples were calculated relative to this mean.

### E. Histology, Immunofluorescence Staining, and Quantifications

For immunofluorescence staining of murine skin, frozen unfixed mouse skin was sectioned, fixed with cold acetone for 10 minutes, and stained as indicated (15). Epidermal activated caspase-3+ cells per high powered field (40× magnification) were quantified by a blinded observer using ImageJ software (NIH) and classified as activated caspase-3+ keratinocytes (Langerin− and CD3−), LCs (Langerin+), or T cells (CD3+).

Formalin-fixed paraffin embedded murine skin was stained with hematoxylin and eosin, and epidermal thickness was measured by a blinded observer using ImageJ software.

For immunofluorescence staining of cell culture experiments, polystyrene chamber slides (Lab-Tek) with cultured keratinocytes were washed with PBS, fixed with 4% paraformaldehyde for 20 minutes, permeabilized and blocked with Triton-X (0.2%) and BSA (1%), and stained as indicated. Activated caspase-3+ and total DAPI+ cells were quantified with ImageJ software by a blinded observer and the percent of activated caspase-3+ Langerin− cells (keratinocytes) and activated caspase-3+ Langerin+ cells (LCs) was calculated.

For immunofluorescence staining of human skin, formalin-fixed paraffin-embedded tissue sections were rehydrated and underwent antigen retrieval at 60° C. in 10 mM citrate buffer, pH 6.0 for 20 hours followed by enzymatic retrieval with Carezyme III: Pronase Kit (Biocare Medical) for 15 minutes. Sections were then stained as indicated. The fluorescence intensity of phosphoEGFR and total EGFR was measured using ImageJ software and the fluorescence intensity of the isotype control was subtracted. The ratio of phosphoEGFR:total EGFR was then calculated and normalized to the ratio for the healthy control samples that were stained at the same time as the SLE samples. Langerin+ cells in the epidermis were counted by a blinded observer using ImageJ software and normalized to the length of the tissue.

All antibodies and staining reagents are described in Table S1 and Table S2 above. Histology was imaged using either a Nikon Eclipse E600 with a Q-Imaging Retiga Exi camera or a Nikon Eclipse NI-E Fluorescence Upright microscope coupled to a Zyla sCMOS camera (Andor Technology).

### F. Western Blots

Western blots were performed essentially as described (23). Ears were harvested and the epidermis was isolated by incubating skin in distilled water at 60° C. for 20 seconds and then in ice cold PBS for 20 seconds before the epidermis was gently scraped off. Epidermal sheets were then lysed on ice with a Polytron PT 10-35 tissue homogenizer in lysis buffer (50 mM Tris-HCl pH 7.7, 1% Triton-X, 150 mM NaCl, 1 mM EDTA, 10 mM NaF, 5 mM β-glycerophosphate, 2 mM NaVO3, 1 mM 1,10-ortho-phenanthroline (Sigma-Aldrich), and protease activator cocktail set III (EMD Millipore)). Samples (10-15 μg protein) were separated on a 10% SDS-polyacrylamide gel, transferred to nitrocellulose paper, and Western blots were then stained as indicated. Antibody staining was detected using ECL Plus Western blotting substrate (Thermo Fisher Scientific). Blots were first stained for phosphoEGFR, stripped with 1 M Tris pH 6.75, β-mercaptoethanol, and SDS at room temperature followed by incubation at 60° C., and then reprobed for total EGFR. All antibodies used for Western blots are described in Table S1 and Table S2. Western blots were quantified with ImageJ software and the ratio of phosphoEGFR: total EGFR was determined and normalized to the ratio of the control samples.

### G. Epidermal Permeability Measurement

Toluidine blue dye penetrance was measured essentially as described (23). Dehydrated and rehydrated ear skin was incubated for 2 min in 0.1% toluidine blue dye (Sigma-Aldrich) before destaining and toluidine blue dye extraction with a solution of 2.5% H2SO4, 2.5% H2O, and 95% methanol. Colorimetric values were measured at 620 nm and the total amount of toluidine blue dye was calculated using the volume of extraction solution, which was constant among the conditions.

### EXAMPLE 9: IN VITRO EXPERIMENTS

### A. Mouse Keratinocyte-LC Co-Cultures:

Primary mouse keratinocyte cultures were prepared from mouse tail skin as described (23). The isolated epidermal cells were plated in 8-well chamberslides (Lab-Tek) coated with 7 ng/4 collagen I (BD Biosciences) at 2-4×105 cells per well in serum-free keratinocyte growth media 2 (KGM2) (PromoCell). 3-4 days later, keratinocytes were at 90% confluency and sorted LCs were added at a density of 20,000-25,000 LCs per well. The co-cultures rested overnight and were then exposed to UVR and analyzed 24 hours later. Unless indicated, co-cultures were exposed to UVR in approximately 200 μL of minimally colored culture media containing 3.3 mM phenol red and without a plastic covering.

### B. Keratinocyte EGFR Knockdown Co-Cultures:

Primary mouse keratinocytes were cultured as described above and, at 40-50% confluency, were treated with control siRNA or two different EGFR siRNAs (siRNA #1 or #2) (Accell siRNA from Dharmacon, GE Lifesciences) according to the manufacturer's protocol. Target sequences of the siRNAs were:

siRNA #1-5′-GAUUGGUGCUGUGCGAUUC-3′ SEQ ID NO: 1 and

siRNA #2-5′-GCAUAGGCAUUGGUGAAUU-3′ SEQ ID NO: 2. The media was changed to normal keratinocyte growth media on day 4, and the co-culture experiments were subsequently conducted as described in Materials and Methods in the main text. Separate wells on the same chamberslides were collected on day 5 (the day of UVR exposure) to check efficiency of EGFR knockdown by flow cytometry.

### C. Keratinocyte PD168393 Treatment Co-Cultures:

Primary mouse keratinocytes were treated with 2 μM PD168393 (Cayman Chemicals), an irreversible EGFR activator, for 30 minutes. The PD168393 was washed off with PBS and fresh keratinocyte growth media was supplied with or without LCs and the co-culture experiments were subsequently conducted. Keratinocytes from separate wells were collected at the time of co-culture and treated with EGF (200 ng/mL) to validate the efficiency of EGFR activation by measuring phosphoEGFR by flow cytometry.

### D. Human Leratinocyte-LC Co-Cultures:

Primary human keratinocytes (Lonza) were prepared according to the manufacturer's protocol and plated on collagen-coated chamberslides at 1-2 days before use. Human LCs and non-LC CD45+ cells were sorted from epidermis and added to 50-90% confluent keratinocyte cultures at 16,000-20,000 cells per chamberslide well. The co-cultures were rested overnight, exposed to UVR, and examined 24 hours after UVR. Some wells of keratinocytes were treated with recombinant human HB-EGF (R&D Systems) at indicated concentrations, rested overnight, and then exposed to UVR. For anti-ADAM17 blocking experiments, LCs were pretreated with 200 nM of anti-human ADAM17 blocking antibody (Abcam) or human IgG1 isotype control antibody (Adipogen) for 30 minutes before they were added with the antibodies to the keratinocytes. Anti-ADAM17 blocking antibody and IgG1 isotype control antibody was also included in keratinocyte cultures without LCs as additional controls.

### EXAMPLE 10: EX VIVO ADAM17 ACTIVITY ASSAY BY TNFR1 CLEAVAGE

Sorted mouse LCs were plated in a 96-well plate at 20,000-25,000 cells/well in RPMI 1640 supplemented with L-glutamine, penicillin/streptomycin, and HEPES buffer. The cells were treated with phorbol 12-myristate 13-acetate (PMA) (Sigma-Aldrich) at 25 ng/mL or UVR (500 J/m2) and analyzed 45 minutes later. The cells were then stained with DAPI to exclude dead cells and for cell-surface TNFR1 (BioLegend). ADAM17 activity is expressed as the percent change in TNFR1 mean fluorescence intensity (MFI) relative to that of untreated LCs. Sorted human LCs (3,000-5,000 cells/well) were plated and treated with anti-ADAM17 blocking antibody or human IgG1 isotype control antibody for 30 minutes prior to UVR exposure and analysis of TNFR1 MFI. For collection of conditioned supernatant from sorted human LCs to add to A431 cells, 1,500 LCs were treated with IgG control or anti-ADAM17 blocking antibody for 30 minutes, washed, and then transferred into new media prior to UVR treatment to prevent carryover of blocking antibodies into the conditioned supernatant.

### EXAMPLE 11: EGFR LIGAND RELEASE ASSAY WITH A431 INDICATOR CELLS

A431 human squamous carcinoma cells (ATCC) were cultured according to the manufacturer's protocol in 96 well plates. At about 80% confluency, the A431 cells were serum-starved overnight then pretreated with 2 mM NaVO3 for 15 minutes at 37° C. and were then treated with conditioned supernatants from various cells for 10 minutes. The A431 cells were then collected and phosphoEGFR expression was measured by flow cytometry. All experiments were conducted with A431 cells in passage 2.

### EXAMPLE 12: LESION QUANTIFICATION

The remaining hair on the back skin of the mice was removed using Nair and photographs were taken. The total back area and lesional area (skin affected by erythema, scaliness, crustiness, or epidermal erosion) was measured by a blinded observer using ImageJ software and skin lesions were quantified as percent of back area.

### EXAMPLE 13: mRNA QUANTIFICATION

Cells were sorted directly into RLT lysis buffer (Qiagen) with β-mercaptoethanol (Bio-Rad) and stored at −80° C. until RNA extraction with Qiagen RNeasy Mini Kit. cDNA was generated using iScript cDNA synthesis kit (Bio-Rad) and real-time PCR was performed using iQ SYBR Green Supermix kit (Bio-Rad) on a Bio-Rad MyiQ thermal cycler or Maxima SYBR Green/ROX qPCR Master Mix (Thermo Fisher Scientific) on a StepOne Plus Real-Time PCR system (Applied Biosystems). qPCR gene expression was quantified relative to Gapdh. Primer sequences used were:

| | | SEQ |
|---|---|---|
| | SEQUENCE | ID NO: |
| MOUSE 5′-3′ | | |
| PRIMER | | |
| Epgn forward | TGGGGGTTCTGATAGCAGTC | 3 |
| Epgn reverse | GGATCACCTCTGCTTCTTCG | 4 |
| Egf forward | CCTGGGAATGTGATTGCTTT | 5 |
| Egf reverse | CCTGGGAATTTGCAAACAGT | 6 |
| Hbegf forward | CCACCTCACTCCCTTTGTGT | 7 |
| Hbegf reverse | AAAGCTCCCTGCTCTTCCTC | 8 |
| Tgfa forward | AAGGCATCTTGGGACAACAC | 9 |
| Tgfa reverse | GCAGGCAGCTTTATCACACA | 10 |
| Btc forward | GGGTGTTTCCCTGCTCTGTA | 11 |
| Btc reverse | TGGATGAGTCCTCAGGTTCC | 12 |
| Areg forward | CATTATGCAGCTGCTTTGGA | 13 |
| Areg reverse | TTTCGCTTATGGTGGAAACC | 14 |
| Ereg forward | CGCTGCTTTGTCTAGGTTCC | 15 |
| Ereg reverse | GGGATCGTCTTCCATCTGAA | 16 |
| Adam17 forward | GATGCTGAAGATGACACTGTG | 17 |
| Adam17 reverse | GAGTTGTCAGTGTCAACGC | 18 |
| Gapdh forward | ATGTGTCCGTCGTGGATCTGA | 19 |
| Gapdh reverse | TTGAAGTCGCAGGAGACAACCT | 20 |
| HUMAN 5′-3′ | | |
| PRIMER | | |
| Epgn forward | ATGACAGCACTGACCGAAGAG | 21 |
| Epgn reverse | AACTGTCCAGTTACCTTGCTG | 22 |
| Egf forward | TCTCAACCCCTTGTACTTTGG | 23 |
| Egf reverse | CAAGTCATCCTCCCATCACCA | 24 |
| Hbegf forward | TTGTGCTCAAGGAATCGGCT | 25 |
| Hbegf reverse | CAACTGGGGACGAAGGAGTC | 26 |
| Tgfa forward | TCGTGAGCCCTCGGTAAGTA | 27 |
| Tgfa reverse | GACTGGTCCCCCTTTCATGG | 28 |
| Btc forward | AAAGCGGAAAGGCCACTTCT | 29 |
| Btc reverse | AGCCTTCATCACAGACACAGG | 30 |
| Areg forward | TGTCGCTCTTGATATCGGC | 31 |
| Areg reverse | ATGGTTCACGCTTCCCAGAG | 32 |
| Ereg forward | TACTGCAGGTGTGAAGTGGG | 33 |
| Ereg reverse | GTGGAACCGACGACTGTGAT | 34 |
| Adam17 forward | TGATGAGCCAGCCAGGAGAT | 38 |
| Adam17 reverse | TATCAAGTCTTGTGGGGACAGC | 35 |
| Gapdh forward | CGACAGTCAGCCGCATCTT | 36 |
| Gapdh reverse | ATCCGTTGACTCCGACCTTC | 37 |

Skin histopathology scoring: A blinded expert dermatopathologist scored H&E stained sections based on dermal inflammation (0-3).

Technical and scientific terms used herein have the same meaning as commonly understood by one of ordinary skill in the art to which this invention belongs and by reference to published texts, which provide one skilled in the art with a general guide to many of the terms used in the present application. The definitions contained in this specification are provided for clarity in describing the components and compositions herein and are not intended to limit the claimed invention.

Each and every patent, patent application, and publication, including websites cited throughout specification are incorporated herein by reference. Similarly, the SEQ ID NOs which are referenced herein, and which appear in the appended Sequence Listing are incorporated by reference. While the invention has been described with reference to particular embodiments, it will be appreciated that modifications can be made without departing from the spirit of the invention. Such modifications are intended to fall within the scope of the appended claims.

### REFERENCES

1. M. K. Kuechle, K. B. Elkon, Shining light on lupus and UV. Arthritis Res Ther 9, 101 (2007).
2. B. F. Chong, V. P. Werth, in Dubois' Lupus Erythematosus and Related Syndromes (Eighth Edition), B. H. Hahn, Ed. (W.B. Saunders, Philadelphia, 2013), pp. 319-332.
3. T. P. Millard, J. L. M. Hawk, Photosensitivity disorders: cause, effect, and management. Am J Clin Dermatol 3, 239-246 (2002).
4. T. D. Golan, et al., Enhanced membrane binding of autoantibodies to cultured keratinocytes of systemic lupus erythematosus patients after ultraviolet B/ultraviolet A irradiation. J Clin Invest 90, 1067-1076 (1992).
5. A. Kuhn, J. et al, Photosensitivity, Apoptosis, and Cytokines in the Pathogenesis of Lupus Erythematosus: a Critical Review. Clin Rev Allerg Immu, 1-15 (2014).
6. E. Reefman et al., Is disturbed clearance of apoptotic keratinocytes responsible for UVB-induced inflammatory skin lesions in systemic lupus erythematosus? Arthritis Res Ther 8, R156 (2006).
7. B. M. Lichtenberger, et al, Epidermal EGFR controls cutaneous host defense and prevents inflammation. Sci Transl Med 5, 199ra111 (2013).
8. D. Raj, et al, Keratinocyte apoptosis in epidermal development and disease. J Invest Dermatol 126, 243-257 (2006).
9. S. W. Kashem, et al, Antigen-Presenting Cells in the Skin. Annu Rev Immunol 35, 469-499 (2017).
10. T. Doebel et al, Langerhans Cells—The Macrophage in Dendritic Cell Clothing. Trends Immunol, 817-828 (2017).
11. R. D. Sontheimer, B. Pr, Epidermal Langerhans cell involvement in cutaneous lupus erythematosus. J Invest Dermatol 79, 237-243 (1982).
12. L. L. Teichmann, et al, Dendritic cells in lupus are not required for activation of T and B cells but promote their expansion, resulting in tissue damage. Immunity 33, 967-978 (2010).
13. J. K. King, et al, Langerhans Cells Maintain Local Tissue Tolerance in a Model of Systemic Autoimmune Disease. J Immunol 195, 464-476 (2015).
14. J. J. Chia, et al, Dendritic cells maintain dermal adipose-derived stromal cells in skin fibrosis. J Clin Invest 126, 4331-4345 (2016).
15. V. Kumar, et al, A dendritic-cell-stromal axis maintains immune responses in lymph nodes. Immunity 42, 719-730 (2015).
16. S. Ivanov, et al, CCR7 and IRF4-dependent dendritic cells regulate lymphatic collecting vessel permeability. J Clin Invest 126, 1581-1591 (2016).
17. J. L. Astarita, et al, The CLEC-2-podoplanin axis controls the contractility of fibroblastic reticular cells and lymph node microarchitecture. Nat Immunol 16, 75-84 (2015).
18. S. E. Acton, et al, Dendritic cells control fibroblastic reticular network tension and lymph node expansion. Nature 514, 498-502 (2014).
19. D. H. Kaplan, et al, Epidermal langerhans cell-deficient mice develop enhanced contact hypersensitivity. Immunity 23, 611-620 (2005).
20. L. Polla, et al, Enhancement of the Elicitation Phase of the Murine Contact Hypersensitivity Response by Prior Exposure to Local Ultraviolet Radiation. J Invest Dermatol 86, 13-17 (1986).
21. T. B. El-Abaseri, et al, Ultraviolet irradiation induces keratinocyte proliferation and epidermal hyperplasia through the activation of the epidermal growth factor receptor. Carcinogenesis 27, 225-231 (2006).
22. A. Haratake, et al, UVB-induced alterations in permeability barrier function: roles for epidermal hyperproliferation and thymocyte-mediated response. J Invest Dermatol 108, 769-775 (1997).
23. C. W. Franzke, et al, Epidermal ADAM17 maintains the skin barrier by regulating EGFR ligand-dependent terminal keratinocyte differentiation. J Exp Med 209, 1105-1119 (2012).
24. S. Tamoutounour, et al, Origins and functional specialization of macrophages and of conventional and monocyte-derived dendritic cells in mouse skin. Immunity 39, 925-938 (2013).
25. T. M. Hohl, et al, Inflammatory monocytes facilitate adaptive CD4 T cell responses during respiratory fungal infection. Cell Host Microbe 6, 470-481 (2009).
26. J. L. Doerner, et al, TWEAK/Fn14 signaling involvement in the pathogenesis of cutaneous disease in the MRL/lpr model of spontaneous lupus. J Invest Dermatol 135, (2015).
27. M. S. Iordanov, et al, The UV (Ribotoxic) Stress Response of Human Keratinocytes Involves the Unexpected Uncoupling of the Ras-Extracellular Signal-Regulated Kinase Signaling Cascade from the Activated Epidermal Growth Factor Receptor. Mol Cell Biol 22, 5380-5394 (2002).
28. F. Mascia, et al, Genetic ablation of epidermal EGFR reveals the dynamic origin of adverse effects of anti-EGFR therapy. Sci Transl Med 5, 199ra110 (2013).
29. D. W. Fry, et al, Specific, irreversible inactivation of the epidermal growth factor receptor and erbB2, by a new class of tyrosine kinase inhibitor. Proc Natl Acad Sci 95, 12022-12027 (1998).
30. T. Ronanet al, Different Epidermal Growth Factor Receptor (EGFR) Agonists Produce Unique Signatures for the Recruitment of Downstream Signaling Proteins. J Biol Chem 291, 5528-5540 (2016).
31. J. Scheller, et al, ADAM17: a molecular switch to control inflammation and tissue regeneration. Trends Immunol 32, 380-387 (2011).
32. K. Horiuchi, et al, Cutting edge: TNF-alpha-converting enzyme (TACE/ADAM17) inactivation in mouse myeloid cells prevents lethality from endotoxin shock. J Immunol 179, 2686-2689 (2007).
33. D. H. Kaplan, et al, Autocrine/paracrine TGFβ1 is required for the development of epidermal Langerhans cells. J Exp Med 204, 2545-2552 (2007).
34. A. Bobr, et al, Autocrine/paracrine TGF-beta1 inhibits Langerhans cell migration. Proc Natl Acad Sci 109, 10492-10497 (2012).
35. B. Singh, et al, UV-induced EGFR signal transactivation is dependent on proligand shedding by activated metalloproteases in skin cancer cell lines. Int J Cancer 124, 531-539 (2009).
36. J. Menke, et al, Sunlight Triggers Cutaneous Lupus through a CSF-1-Dependent Mechanism in MRL-Faslpr Mice. J Immunol 181, 7367-7379 (2008).
37. Y. Horiguchi, et al, Effects of ultraviolet light irradiation on the skin of MRL/l mice. Arch Dermatol Res 279, 478-483 (1987).
38. A. U. Eriksson, R. R. Singh, Cutting edge: migration of langerhans dendritic cells is impaired in autoimmune dermatitis. J Immunol 181, 7468-7472 (2008).
39. P. Pisitkun, et al, Autoreactive B cell responses to RNA-related antigens due to TLR7 gene duplication. Science 312, 1669-1672 (2006).
40. L. Morel, et al, Genetic reconstitution of systemic lupus erythematosus immunopathology with polycongenic murine strains. Proc Natl Acad Sci 97, 6670-6675 (2000).
41. R. P. Huang, et al, UV activates growth factor receptors via reactive oxygen intermediates. The Journal of Cell Biology 133, 211-220 (1996).
42. C. Sachsenmaier, et al, Involvement of growth factor receptors in the mammalian UVC response. Cell 78, 963-972 (1994).
43. Y. Xu, et al, Oxidative Inhibition of Receptor-type Protein-tyrosine Phosphatase κ by Ultraviolet Irradiation Activates Epidermal Growth Factor Receptor in Human Keratinocytes. Journal of Biological Chemistry 281, 27389-27397 (2006).
44. L. A. Monticelli, et al, IL-33 promotes an innate immune pathway of intestinal tissue protection dependent on amphiregulin-EGFR interactions. Proc Natl Acad Sci USA 112, 10762-10767 (2015).
45. N. Arpaia, et al, A Distinct Function of Regulatory T Cells in Tissue Protection. Cell 162, 1078-1089 (2015).
46. M. Hatakeyama, et al, Anti-Inflammatory Role of Langerhans Cells and Apoptotic Keratinocytes in Ultraviolet-B-Induced Cutaneous Inflammation. J Immunol, 2937-2947 (2017).
47. K. D. Cooper, et al, UV exposure reduces immunization rates and promotes tolerance to epicutaneous antigens in humans: relationship to dose, CD1a-DR+ epidermal macrophage induction, and Langerhans cell depletion. Proc Natl Acad Sci 89, 8497-8501 (1992).
48. C. Sontheimer, et al, Ultraviolet B Irradiation Causes Stimulator of Interferon Genes-Dependent Production of Protective Type I Interferon in Mouse Skin by Recruited Inflammatory Monocytes. Arthritis Rhematol 69, 826-836 (2017).
49. N. Kollias, et al, The value of the ratio of UVA to UVB in sunlight. Photochem Photobiol 87, 1474-1475 (2011).
50. P. Uribe, S. Gonzalez, Epidermal growth factor receptor (EGFR) and squamous cell carcinoma of the skin: molecular bases for EGFR-targeted therapy. Pathology, research and practice 207, 337-342 (2011).
51. J. U. Shin, et al, Treatment of epidermal growth factor receptor inhibitor-induced acneiform eruption with topical recombinant human epidermal growth factor. Dermatology 225, 135-140 (2012).
52. P. E. Dubé, et al, Epidermal growth factor receptor inhibits colitis-associated cancer in mice. J Clin Invest 122, 2780-2792 (2012).
53. P. M. Izmirly, et al, Dysregulation of the Microvasculature in Nonlesional Non-Sun-exposed Skin of Patients with Lupus Nephritis. J Rheumatol 39, 510-515 (2012).
54. Le Gall S, et al. ADAMs 10 and 17 represent differentially regulated components of a general shedding machinery for membrane proteins such as TGFα, L-Selectin and TNFα. Mol Biol Cell. 2009; 20:1785-1794.
55. Le Gall S M, et al. ADAM17 is regulated by a rapid and reversible mechanism that controls access to its catalytic site. J Cell Science. 2010; 123(22):3913-3922.
56. Blobel C P. ADAMs: key players in EGFR-signaling, development and disease. Nat Rev Mol Cell Bio. 2005; 6:32-43.
57. Maretzky T, et al. Migration of growth factor-stimulated epithelial and endothelial cells depends on EGFR transactivation by ADAM17. Nat Commun. 2011; 2:229.
58. Maretzky T, et al. Src stimulates fibroblast growth factor receptor-2 shedding by an ADAM15 splice variant linked to breast cancer. Cancer Res. 2009; 69(11):4573-4576.
59. Swendeman S, et al. VEGF-A stimulates ADAM17-dependent shedding of VEGFR2 and crosstalk between VEGFR2 and ERK signaling. Circ Res. 2008; 103(9):916-918.
60. Mendelson K, et al. Stimulation of the PDGFR{beta} activates ADAM17 and promotes metalloproteinase-dependent crosstalk between the PDGFR{beta} and EGFR signaling pathways. J Biol Chem. 2010.
61. Yoda M, et al. Systemic Overexpression of TNFalpha-converting Enzyme Does Not Lead to Enhanced Shedding Activity In Vivo. PLoS One. 2013; 8(1):e54412.
62. Adrain C, et al. Tumor necrosis factor signaling requires iRhom2 to promote trafficking and activation of TACE. Science. 2012; 335(6065):225-228.
63. Weskamp G, et al. ADAM17 stabilizes its interacting partner inactive Rhomboid 2 (iRhom2) but not inactive Rhomboid 1 (iRhom1). J Biol Chem. 2020.
64. Haxaire C, et al. Blood-induced bone loss in murine hemophilic arthropathy is prevented by blocking the iRhom2/ADAM17/TNF-alpha pathway. Blood. 2018; 132(10):1064-1074.
65. Inoue A, et al. TGFalpha shedding assay: an accurate and versatile method for detecting GPCR activation. Nat Methods. 2012; 9(10):1021-1029.
66. Inoue A, et al, LPA-producing enzyme PA-PLA(1)alpha regulates hair follicle development by modulating EGFR signalling. EMBO J. 2011; 30(20):4248-4260.
67. Bian G, et al. Sphingosine 1-phosphate stimulates eyelid closure in the developing rat by stimulating EGFR signaling. Sci Signal. 2018; 11(553).
68. Montell C. Preventing a Perm with TRPV3. Cell. 2010; 141(2):218-220.
69. Cheng X, et al. TRP channel regulates EGFR signaling in hair morphogenesis and skin barrier formation. Cell. 2010; 141(2):331-343.
70. Sinha A, et al. Epidermal growth factor enemas with oral mesalamine for mild-to-moderate left-sided ulcerative colitis or proctitis. N Engl J Med. 2003; 349(4):350-357.
71. Dube P E, et al. Pharmacological activation of epidermal growth factor receptor signaling inhibits colitis-associated cancer in mice. Sci Rep. 2018; 8(1):9119.
72. Schultz G S, et al. Epithelial wound healing enhanced by transforming growth factor-alpha and vaccinia growth factor. Science. 1987; 235(4786):350-352.
73. Schultz G, et al, EGF and TGF-alpha in wound healing and repair. J Cell Biochem. 1991; 45(4):346-352.
74. Sakanyan V, et al. Screening and discovery of nitro-benzoxadiazole compounds activating epidermal growth factor receptor (EGFR) in cancer cells. Sci Rep. 2014; 4:3977.
75. Moss, M L and D. Minond, Recent Advances in ADAM17 Research: A Promising Target for Cancer and Inflammation, Mediators of Inflammation, 2017 Art ID 9673537, November 2017, oi.org/10.1155/2017/9673537
76. Wang, S., et al, Febuary 2019, Recent advances on the roles of epidermal growth factor receptor in psoriasis. Am. J. Transl. Res., 2019:11(2):520-528

1. A method of treating or suppressing ultraviolet radiation sensitivity in a subject in need thereof, comprising administering a therapeutically effective amount of an agent which activates or increases the expression or activity of ADAM 17, or activates or increase the release of EGFR ligands, or increases epidermal EGFR in the subject's Langerhans cells.

2. The method according to claim 1, wherein the subject has systemic lupus erythematosus.

3. The method according to claim 1, wherein said agent is lysophosphatidic acid or an analog or derivative thereof.

4. The method according to claim 1, wherein said agent is a P2y5 agonist or an analog or derivative thereof.

5. The method according to claim 1, wherein said agent is a recombinant PA-PLA(1)α enzyme or an analog or derivative thereof.

6. The method according to claim 1, wherein said agent is S1P or an analog or derivative thereof.

7. The method according to claim 1, wherein said agent is TNFα or an analog or derivative thereof.

8. The method according to claim 1, wherein said agent is a TRPV3 ion channel activator an analog or derivative thereof.

9. The method according to claim 1, wherein said agent is an EGFR activator or an analog or derivative thereof.

10. The method according to claim 1, wherein said agent is an TLR activator or an analog or derivative thereof.

11. The method according to claim 1, wherein said agent is a recombinant EGF, an EGFR-agonist, or an analog or derivative thereof

12. The method according to claim 1, wherein said agent is post-transcriptional or transcriptional activator of ADAM17.

13. The method according to claim 1, wherein said agent is recombinant EGF or an hb-EGFR or an analog or derivative thereof.

14. The method according to claim 1, wherein said agent is administered in a pharmaceutically acceptable carrier.

15. The method according to claim 1, wherein said agent is administered topically to the subject.

16. A composition for treating or suppressing ultraviolet radiation sensitivity in a subject in need thereof, comprising a therapeutically effective amount of an agent which activates or increases the expression or activity of ADAM 17, or activates or increase the release of EGFR ligands, or increases epidermal EGFR in the subject's Langerhans cells.

17. The composition according to claim 16, which is a skin cream formulation, a sunscreen formulation, a shampoo formulation, a spray, an ointment, a rinse, or a dry formulation.

18. (canceled)


SEQUENCE LISTING (38 sequences)

SEQ ID NO: 1 - RNA, 19 nt, Mus musculus
gauuggugcugugcgauuc

SEQ ID NO: 2 - RNA, 19 nt, Mus musculus
gcauaggcauuggugaauu

SEQ ID NO: 3 - DNA, 20 bp, Mus musculus
tgggggttctgatagcagtc

SEQ ID NO: 4 - DNA, 20 bp, Mus musculus
ggatcacctctgcttcttcg

SEQ ID NO: 5 - DNA, 20 bp, Mus musculus
cctgggaatgtgattgcttt

SEQ ID NO: 6 - DNA, 20 bp, Mus musculus
cctgggaatttgcaaacagt

SEQ ID NO: 7 - DNA, 20 bp, Mus musculus
ccacctcactccctttgtgt

SEQ ID NO: 8 - DNA, 20 bp, Mus musculus
aaagctccctgctcttcctc

SEQ ID NO: 9 - DNA, 20 bp, Mus musculus
aaggcatcttgggacaacac

SEQ ID NO: 10 - DNA, 20 bp, Mus musculus
gcaggcagctttatcacaca

SEQ ID NO: 11 - DNA, 20 bp, Mus musculus
gggtgtttccctgctctgta

SEQ ID NO: 12 - DNA, 20 bp, Mus musculus
tggatgagtcctcaggttcc

SEQ ID NO: 13 - DNA, 20 bp, Mus musculus
cattatgcagctgctttgga

SEQ ID NO: 14 - DNA, 20 bp, Mus musculus
tttcgcttatggtggaaacc

SEQ ID NO: 15 - DNA, 20 bp, Mus musculus
cgctgctttgtctaggttcc

SEQ ID NO: 16 - DNA, 20 bp, Mus musculus
gggatcgtcttccatctgaa

SEQ ID NO: 17 - DNA, 21 bp, Mus musculus
gatgctgaagatgacactgtg

SEQ ID NO: 18 - DNA, 19 bp, Mus musculus
gagttgtcagtgtcaacgc

SEQ ID NO: 19 - DNA, 21 bp, Mus musculus
atgtgtccgtcgtggatctga

SEQ ID NO: 20 - DNA, 22 bp, Mus musculus
ttgaagtcgcaggagacaacct

SEQ ID NO: 21 - DNA, 21 bp, Homo sapiens
atgacagcactgaccgaagag

SEQ ID NO: 22 - DNA, 21 bp, Homo sapiens
aactgtccagttaccttgctg

SEQ ID NO: 23 - DNA, 21 bp, Homo sapiens
tctcaaccccttgtactttgg

SEQ ID NO: 24 - DNA, 21 bp, Homo sapiens
caagtcatcctcccatcacca

SEQ ID NO: 25 - DNA, 19 bp, Homo sapiens
tgtgctcaaggaatcggct

SEQ ID NO: 26 - DNA, 20 bp, Homo sapiens
caactggggacgaaggagtc

SEQ ID NO: 27 - DNA, 20 bp, Homo sapiens
tcgtgagccctcggtaagta

SEQ ID NO: 28 - DNA, 20 bp, Homo sapiens
gactggtccccctttcatgg

SEQ ID NO: 29 - DNA, 20 bp, Homo sapiens
aaagcggaaaggccacttct

SEQ ID NO: 30 - DNA, 21 bp, Homo sapiens
agccttcatcacagacacagg

SEQ ID NO: 31 - DNA, 19 bp, Homo sapiens
tgtcgctcttgatatcggc

SEQ ID NO: 32 - DNA, 20 bp, Homo sapiens
atggttcacgcttcccagag

SEQ ID NO: 33 - DNA, 20 bp, Homo sapiens
tactgcaggtgtgaagtggg

SEQ ID NO: 34 - DNA, 20 bp, Homo sapiens
gtggaaccgacgactgtgat

SEQ ID NO: 35 - DNA, 22 bp, Homo sapiens
tatcaagtcttgtggggacagc

SEQ ID NO: 36 - DNA, 19 bp, Homo sapiens
cgacagtcagccgcatctt

SEQ ID NO: 37 - DNA, 20 bp, Homo sapiens
atccgttgactccgaccttc

SEQ ID NO: 38 - DNA, 20 bp, Homo sapiens
tgatgagccagccaggagat